SEQUENCE LISTING (131 sequences)

SEQ ID NO: 1 - protein, 107 aa, Homo sapiens [sequence omitted]

SEQ ID NO: 2 - protein, 104 aa, Homo sapiens
  MISC_FEATURE (1)..(104): Human IgG CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
  MISC_FEATURE (1)..(98): Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
  MISC_FEATURE (1)..(98): Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 5 - protein, 98 aa, Homo sapiens
  MISC_FEATURE (1)..(98): Human IgG3 CH1 Domain
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 6 - protein, 98 aa, Homo sapiens
  MISC_FEATURE (1)..(98): Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 7 - protein, 15 aa, Homo sapiens
  MISC_FEATURE (1)..(15): Human IgG1 Hinge Domain
EPKSCDKTHTCPPCP

SEQ ID NO: 8 - protein, 12 aa, Homo sapiens
  MISC_FEATURE (1)..(12): Human IgG2 Hinge Domain
ERKCCVECPPCP

SEQ ID NO: 9 - protein, 62 aa, Homo sapiens
  MISC_FEATURE (1)..(62): Human IgG3 Hinge Domain
ELKTPLGDTTHTCPRCPEPKSCDTPPPCPRCPEPKSCDTPPPCPRCPEPKSCDTPPPCPRCP

SEQ ID NO: 10 - protein, 12 aa, Homo sapiens
  MISC_FEATURE (1)..(12): Human IgG4 Hinge Domain
ESKYGPPCPSCP

SEQ ID NO: 11 - protein, 12 aa, Artificial Sequence
  IgG4 Hinge Domain Comprising the Stabilizing S228P Substitution
ESKYGPPCPPCP

SEQ ID NO: 12 - protein, 217 aa, Homo sapiens
  MISC_FEATURE (1)..(217): Exemplary Human IgG1 CH2-CH3 Domain
  MISC_FEATURE (217)..(217): Xaa is lysine (K) or is Absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 13 - protein, 216 aa, Homo sapiens
  MISC_FEATURE (1)..(216): Exemplary Human IgG2 CH2-CH3 Domain
  MISC_FEATURE (216)..(216): Xaa is lysine (K) or is Absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 14 - protein, 217 aa, Homo sapiens
  ′MISC_FEATURE (1)..(217): Exemplary Human IgG3 CH2-CH3 Domain
  ′MISC_FEATURE (217)..(217): Xaa is lysine (K) or is Absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 15 - protein, 217 aa, Homo sapiens
  MISC_FEATURE (1)..(217): Exemplary Human IgG4 CH2-CH3 Domain
  MISC_FEATURE (217)..(217): Xaa is lysine (K) or is Absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 16 - protein, 8 aa, Artificial Sequence
  Preferred Intervening Spacer Peptide (Linker 1)
GGGSGGGG

SEQ ID NO: 17 - protein, 6 aa, Artificial Sequence
  Preferred Cysteine-Containing Spacer Peptide (Linker 2)
GGCGGG

SEQ ID NO: 18 - protein, 4 aa, Artificial Sequence
  Alternative Non-Cysteine-Containing Linker 2 Spacer Peptide
GGGS

SEQ ID NO: 19 - protein, 6 aa, Artificial Sequence
  Alternative Non-Cysteine-Containing Linker 2 Spacer Peptide
LGGGSG

SEQ ID NO: 20 - protein, 11 aa, Artificial Sequence
  Alternative Non-Cysteine-Containing Linker 2 Spacer Peptide
GGGSGGGSGGG

SEQ ID NO: 21 - protein, 5 aa, Artificial Sequence
  Alternative Non-Cysteine-Containing Linker 2 Spacer Peptide
ASTKG

SEQ ID NO: 22 - protein, 6 aa, Artificial Sequence
  Alternative Non-Cysteine-Containing Linker 2 Spacer Peptide
LEPKSS

SEQ ID NO: 23 - protein, 5 aa, Artificial Sequence
  Alternative Non-Cysteine-Containing Linker 2 Spacer Peptide
APSSS

SEQ ID NO: 24 - protein, 7 aa, Artificial Sequence
  Heterodimer-Promoting Domain
GVEPKSC

SEQ ID NO: 25 - protein, 6 aa, Artificial Sequence
  Heterodimer-Promoting Domain
VEPKSC

SEQ ID NO: 26 - protein, 6 aa, Artificial Sequence
  Heterodimer-Promoting Domain
AEPKSC

SEQ ID NO: 27 - protein, 7 aa, Artificial Sequence
  Heterodimer-Promoting Domain
GFNRGEC

SEQ ID NO: 28 - protein, 6 aa, Artificial Sequence
  Heterodimer-Promoting Domain
FNRGEC

SEQ ID NO: 29 - protein, 28 aa, Artificial Sequence
  “E-coil” Heterodimer-Promoting Domain
EVAALEKEVAALEKEVAALEKEVAALEK

SEQ ID NO: 30 - protein, 28 aa, Artificial Sequence
  “K-coil” Heterodimer-Promoting Domain
KVAALKEKVAALKEKVAALKEKVAALKE

SEQ ID NO: 31 - protein, 28 aa, Artificial Sequence
  Cysteine-Containing “E-coil” Heterodimer- Promoting Domain
EVAACEKEVAALEKEVAALEKEVAALEK

SEQ ID NO: 32 - protein, 28 aa, Artificial Sequence
  Cysteine-Containing “K-coil” Heterodimer- Promoting Domain
KVAACKEKVAALKEKVAALKEKVAALKE

SEQ ID NO: 33 - protein, 46 aa, Streptococcus dysgalactiae
  MISC_FEATURE (1)..(46): Albumin-Binding Domain 3 (ABD3) of Protein G of Streptococcus strain G148
LAEAKVLANRELDKYGVSDYYKNLIDNAKSAEGVKALIDEILAALP

SEQ ID NO: 34 - protein, 46 aa, Artificial Sequence; identical to SEQ ID NO: 33
  N66D / T70S / V71A Variant Deimmunized Albumin-Binding Domain 3 (ABD3) of Protein G of Streptococcus strain G148

SEQ ID NO: 35 - protein, 46 aa, Artificial Sequence
  L64A / I65A / D79A Variant Deimmunized Albumin-Binding Domain 3 (ABD3) of Protein G of Streptococcus strain G148
LAEAKVLANRELDKYGVSDYYKNAANNAKTVEGVKALIAEILAALP

SEQ ID NO: 36 - protein, 46 aa, Artificial Sequence
  N66S / T70S / D79A Variant Deimmunized Albumin-Binding Domain 3 (ABD3) of Protein G of Streptococcus strain G148
LAEAKVLANRELDKYGVSDYYKNLISNAKSVEGVKALIAEILAALP

SEQ ID NO: 37 - protein, 8 aa, Artificial Sequence
  Intervening Spacer Peptide (Linker)
APSSSPME

SEQ ID NO: 38 - protein, 16 aa, Artificial Sequence
  Intervening Spacer Peptide (Linker)
VEPKSADKTHTCPPCP

SEQ ID NO: 39 - protein, 16 aa, Artificial Sequence
  Intervening Spacer Peptide (Linker)
LEPKSADKTHTCPPCP

SEQ ID NO: 40 - protein, 10 aa, Artificial Sequence
  Intervening Spacer Peptide (Linker)
DKTHTCPPCP

SEQ ID NO: 41 - protein, 15 aa, Artificial Sequence
  scFv Spacer Peptide Linker
GGGGSGGGGSGGGGS

SEQ ID NO: 42 - protein, 12 aa, Artificial Sequence
  “Long” Intervening Spacer Peptide Linker
GGGGSGGGSGGG

SEQ ID NO: 43 - protein, 13 aa, Artificial Sequence
  Alternative Intervening Spacer Peptide (Linker)
GGGDKTHTCPPCP

SEQ ID NO: 44 - protein, 16 aa, Artificial Sequence
  Alternative Intervening Spacer Peptide (Linker)
LEPKSSDKTHTCPPCP

SEQ ID NO: 45 - protein, 217 aa, Artificial Sequence
  Exemplary Human IgG1 CH2-CH3 Domain Having L234A/L235A Substitutions
  MISC_FEATURE (217)..(217): Xaa is lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 46 - protein, 217 aa, Artificial Sequence
  Exemplary Human IgG1 CH2-CH3 Domain Having M252Y/S254T/T256E Substitutions
  MISC_FEATURE (217)..(217): Xaa is lysine (K) or is Absent
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 47 - protein, 217 aa, Artificial Sequence
  Exemplary Human IgG1 CH2-CH3 Domain Having L234A/L235A/M252Y/S254T/T256E Substitutions
  MISC_FEATURE (217)..(217): Xaa is lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 48 - protein, 217 aa, Artificial Sequence
  Exemplary “Knob-Bearing” Human IgG1 CH2-CH3 Domain Having L234A/L235A Substitutions
  MISC_FEATURE (217)..(217): Xaa is lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 49 - protein, 217 aa, Artificial Sequence
  Exemplary “Knob-Bearing” Human IgG1 CH2-CH3 Domain Having L234A/L235A/M252Y/S254T/T256E Substitutions
  MISC_FEATURE (217)..(217): Xaa is lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 50 - protein, 217 aa, Artificial Sequence
  Exemplary “Hole-Bearing” Human IgG1 CH2-CH3 Domain Having L234A/L235A Substitutions
  MISC_FEATURE (217)..(217): XAA is Lysine (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 51 - protein, 217 aa, Artificial Sequence
  Exemplary “Hole-Bearing” Human IgG1 CH2-CH3 Domain Having L234A/L235A/M252Y/S254T/T256E Substitutions
  MISC_FEATURE (217)..(217): Xaa is lysine (K) or is Absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 52 - protein, 217 aa, Artificial Sequence
  Exemplary Human IgG4 CH2-CH3 Domain Having M252Y/S254T/T256E Substitutions
  MISC_FEATURE (217)..(217): Xaa is lysine (K) or is Absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 53 - protein, 217 aa, Artificial Sequence
  Exemplary “Knob-Bearing” Human IgG4 CH2-CH3 Domain Having M252Y/S254T/T256E Substitutions
  MISC_FEATURE (217)..(217): Xaa is lysine (K) or is Absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 54 - protein, 217 aa, Artificial Sequence
  Exemplary “Hole-Bearing” Human IgG4 CH2-CH3 Domain Having M252Y/S254T/T256E Substitutions
  MISC_FEATURE (217)..(217): Xaa is lysine (K) or is Absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGX

SEQ ID NO: 55 - protein, 113 aa, Homo sapiens
  MISC_FEATURE (1)..(113): VL Domain of Anti-HIV Env Human IgG1 Antibody 7B2
DIVMTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVEIK

SEQ ID NO: 56 - protein, 126 aa, Homo sapiens
  MISC_FEATURE (1)..(126): VH Domain of Anti-HIV Env Human IgG1 Antibody 7B2
QVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSS

SEQ ID NO: 57 - protein, 113 aa, Artificial Sequence
  VL Domain of Optimized Anti-HIV Env Human IgG1 Antibody 7B2GL
DIVMTQSPDSLAVSLGERATINCKSSQTLLYSSNNRHSIAWYQQKPGQPPKLLIYWASMRLSGVPDRFSGSGSGTDFTLTISSLQAEDVAVYYCHQYSSHPPTFGQGTKVEIK

SEQ ID NO: 58 - protein, 126 aa, Artificial Sequence
  VH Domain of Optimized Anti-HIV Env Human IgG1 Antibody 7B2GL
QVQLVESGGGLVKPGGSLRLSCAASGFTFSEYYMTWIRQAPGKGLEWVSYISKNGEYSKYSPSSNGRFTISRDNAKNSLYLQMNSLRAEDTAVYYCARADGLTYFSELLQYIFDLWGQGTLVTVSS

SEQ ID NO: 59 - protein, 118 aa, Mus musculus
  MISC_FEATURE (1)..(118): VH Domain of Anti-Human CD2 mAb Lo-CD2a
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 60 - protein, 112 aa, Mus musculus
  MISC_FEATURE (1)..(112): VL Domain of Anti-Human CD2 mAb Lo-CD2a
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 61 - protein, 125 aa, Artificial Sequence
  VH Domain of Humanized Anti-Human CD3 mAb 1
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 62 - protein, 110 aa, Artificial Sequence
  VL Domain of Humanized Anti-Human CD3 mAb 1
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 63 - protein, 125 aa, Artificial Sequence
  VH Domain of Humanized Anti-Human CD3 mAb 1 (D65G)
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 64 - protein, 125 aa, Artificial Sequence
  VH Domain of Humanized Anti-Human CD3 mAb 1 Low
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSS

SEQ ID NO: 65 - protein, 125 aa, Artificial Sequence
  VH Domain of Humanized Anti-Human CD3 mAb 1 Fast
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSS

SEQ ID NO: 66 - protein, 119 aa, Mus musculus [sequence omitted]

SEQ ID NO: 67 - protein, 107 aa, Mus musculus
  MISC_FEATURE (1)..(107): VL Domain of Anti-Human CD3 Antibody OKT3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 68 - protein, 120 aa, Mus musculus
  MISC_FEATURE (1)..(120): VH Domain of Anti-Human CD8 Antibody OKT8
QVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSS

SEQ ID NO: 69 - protein, 112 aa, Mus musculus
  MISC_FEATURE (1)..(112): VL Domain of Anti-Human CD8 Antibody OKT8
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKR

SEQ ID NO: 70 - protein, 121 aa, Artificial Sequence
  VH Domain of Humanized Anti-Human CD8 Antibody TRX2
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 71 - protein, 106 aa, Artificial Sequence
  VL Domain of Humanized Anti-Human CD8 Antibody TRX2
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 72 - protein, 118 aa, Mus musculus
  MISC_FEATURE (1)..(118): VH Domain of Anti-Human CD16 Antibody 3G8
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 73 - protein, 111 aa, Mus musculus
  MISC_FEATURE (1)..(111): VL Domain of Anti-Human CD16 Antibody 3G8
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 74 - protein, 118 aa, Artificial Sequence
  VH Domain of Humanized Anti-Human CD16 Antibody h3G8
QVTLRESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSS

SEQ ID NO: 75 - protein, 111 aa, Artificial Sequence
  VL Domain of Humanized Anti-Human CD16 Antibody h3G8
DIVMTQSPDSLAVSLGERATINCKASQSVDFDGDSFMNWYQQKPGQPPKLLIYTTSNLESGVPDRFSGSGSGTDFTLTISSLQAEDVAVYYCQQSNEDPYTFGQGTKLEIK

SEQ ID NO: 76 - protein, 117 aa, Mus musculus
  MISC_FEATURE (1)..(117): VH Domain of Anti-Human CD16 Antibody A9
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSS

SEQ ID NO: 77 - protein, 111 aa, Mus musculus
  MISC_FEATURE (1)..(111): VL Domain of Anti-Human CD16 Antibody A9
DIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

SEQ ID NO: 78 - protein, 120 aa, Artificial Sequence
  VH Domain of Anti-Human CD16A Antibody of US Patent 9,035,026
EVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 79 - protein, 106 aa, Artificial Sequence
  First Exemplary VL Domain of Anti-Human CD16A Antibody of US Patent 9,035,026
SYELMQPPSVSVSSGQTASIPCSGDKLEEKYVSWYQQRPGQSPVLVIYQDNKRPSGIPERFSGSNSGNTATLTISGTQAMDEADYYCQVWDNYSVLFGGGTKLTVL

SEQ ID NO: 80 - protein, 106 aa, Artificial Sequence
  Second Exemplary VL Domain of Anti-Human CD16A Antibody of US Patent 9,035,026
SYELTQPLSESVAQGQTARITCGGNNIESRNVHWYQQKPGQAPVLVIYRDNNRPSGIPERFSGSNSGNMATLTISRAQAGDAADYYCQVWDNYTVLFGGGTKLTVL

SEQ ID NO: 81 - protein, 106 aa, Artificial Sequence
  Third Exemplary VL Domain of Anti-Human CD16A Antibody of US Patent 9,035,026
SYELTQPPSVAVAPGKTARITCGGNNIGSKNVHWYQQKPGQAPVLVIYRDSNRPSGIPERFSGSNSGNTATLTISRAQAGDEADFYCQVWDNYIVLFGGGTKLTVL

SEQ ID NO: 82 - protein, 106 aa, Artificial Sequence
  Fourth Exemplary VL Domain of Anti-Human CD16A Antibody of US Patent 9,035,026
QAVLTQPPSVSVAPGQTARIPCEGNNIGSKNVHWYRQKPGQVPVLVMYDDSDRPSGIPERFSGSNSGNTATLTISGTQAMDEADYYCQVWDNYSVLFGGGTKLTVL

SEQ ID NO: 83 - protein, 106 aa, Artificial Sequence
  Fifth Exemplary VL Domain of Anti-Human CD16A Antibody of US Patent 9,035,026
QPVLTQPLSVSVAPGQTARITCGGNNIGSKNVHWYQQKPGQAPVLVIYRDSSRPSGIPERLSGSNSGDTATLTISRAQAGDEADYYCQVWDDYIVVFGGGTKLTVL

SEQ ID NO: 84 - protein, 106 aa, Artificial Sequence
  Sixth Exemplary VL Domain of Anti-Human CD16A Antibody of US Patent 9,035,026
SYELTQPPSVSVTPGQTATITCGANDIGKRNVHWYQQRPGQSPVLVIYQDNKRPSGIPERFSGSNSGNTATLTISGTQAMDEADYYCQVWDNYSVLFGGGTKLTVL

SEQ ID NO: 85 - protein, 106 aa, Artificial Sequence
  Seventh Exemplary VL Domain of Anti-Human CD16A Antibody of US Patent 9,035,026
QPVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 86 - protein, 120 aa, Mus musculus
  MISC_FEATURE (1)..(120): VH Domain of Anti-Human TCR Antibody BMA 031
QVQLVQSGAEVKKPGASVKVSCKASGYKFTSYVMHWVRQAPGQGLEWIGYINPYNDVTKYNEKFKGRVTITADKSTSTAYLQMNSLRSEDTAVHYCARGSYYDYDGFVYWGQGTLVTVSS

SEQ ID NO: 87 - protein, 106 aa, Mus musculus
  MISC_FEATURE (1)..(106): VL Domain of Anti-Human TCR Antibody BMA 031
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 88 - protein, 118 aa, Homo sapiens
  MISC_FEATURE (1)..(118): VH Domain of Human Anti-Human NKG2D Receptor Antibody KYK-1.0
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 89 - protein, 108 aa, Homo sapiens
  MISC_FEATURE (1)..(108): VL Domain of Human Anti-Human NKG2D Receptor Antibody KYK-1.0
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 90 - protein, 121 aa, Homo sapiens
  MISC_FEATURE (1)..(121): VH Domain of Human Anti-Human NKG2D Receptor Antibody KYK-2.0
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 91 - protein, 110 aa, Homo sapiens
  MISC_FEATURE (1)..(110): VL Domain of Human Anti-Human NKG2D Receptor Antibody KYK-2.0
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 92 - protein, 117 aa, Mus musculus
  MISC_FEATURE (1)..(117): VH Domain of Anti-Human NKp46 Antibody BAB281
QIQLVQSGPELQKPGETVKISCKASGYTFTNYGMNWVKQAPGKGLKWMGWINTNTGEPTYAEEFKGRFAFSLETSASTAYLQINNLKNEDTATYFCARDYLYYFDYWGQGTTLTVSS

SEQ ID NO: 93 - protein, 108 aa, Mus musculus
  MISC_FEATURE (1)..(108): VL Domain of Anti-Human NKp46 Antibody BAB281
DNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 94 - protein, 116 aa, Mus musculus
  MISC_FEATURE (1)..(116): VH Domain of Anti-Human NKp46 Antibody NKp46-3
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 95 - protein, 107 aa, Mus musculus
  MISC_FEATURE (1)..(107): VL Domain of Anti-Human NKp46 Antibody NKp46-3
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 96 - protein, 123 aa, Homo sapiens
  MISC_FEATURE (1)..(123): VH Domain of Human Anti-HIV-1 Env gp120 Antibody A32
QVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTLVTVSS

SEQ ID NO: 97 - protein, 110 aa, Homo sapiens
  MISC_FEATURE (1)..(110): VL Domain of Human Anti-HIV-1 Env gp120 Antibody A32
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVL

SEQ ID NO: 98 - protein, 132 aa, Artificial Sequence
  VH Domain of Humanized Anti-HIV-1 Env gp120 Antibody 10-1074
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 99 - protein, 108 aa, Artificial Sequence
  VL Domain of Humanized Anti-HIV-1 Env gp120 Antibody 10-1074
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVL

SEQ ID NO: 100 - protein, 123 aa, Homo sapiens
  MISC_FEATURE (1)..(123): VH Domain of Humanized Anti-HIV-1 Env gp120 Antibody 3BNC117
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSS

SEQ ID NO: 101 - protein, 99 aa, Homo sapiens
  MISC_FEATURE (1)..(99): VL Domain of Humanized Anti-HIV-1 Env gp120 Antibody 3BNC117
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLK

SEQ ID NO: 102 - protein, 132 aa, Homo sapiens
  MISC_FEATURE (1)..(132): VH Domain of Humanized Anti-HIV-1 Env gp120 Antibody PGT121
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 103 - protein, 105 aa, Homo sapiens
  MISC_FEATURE (1)..(105): VL Domain of Humanized Anti-HIV-1 Env gp120 Antibody PGT121
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 104 - protein, 140 aa, Homo sapiens
  MISC_FEATURE (1)..(140): VH Domain of Humanized Anti-HIV-1 Env gp120 Antibody PGT145
QVQLVQSGAEVKKPGSSVKVSCKASGNSFSNHDVHWVRQATGQGLEWMGWMSHEGDKTGLAQKFQGRVTITRDSGASTVYMELRGLTADDTAIYYCLTGSKHRLRDYFLYNEYGPNYEEWGDYLATLDVWGHGTAVTVSS

SEQ ID NO: 105 - protein, 112 aa, Homo sapiens
  MISC_FEATURE (1)..(112): VL Domain of Humanized Anti-HIV-1 Env gp120 Antibody PGT145
EVVITQSPLFLPVTPGEAASLSCKCSHSLQHSTGANYLAWYLQRPGQTPRLLIHLATHRASGVPDRFSGSGSGTDFTLKISRVESDDVGTYYCMQGLHSPWTFGQGTKVEIK

SEQ ID NO: 106 - protein, 121 aa, Homo sapiens
  MISC_FEATURE (1)..(121): VH Domain of Human Anti-HIV-1 Env gp120 Antibody VRC01
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 107 - protein, 104 aa, Homo sapiens
  MISC_FEATURE (1)..(104): VL Domain of Human Anti-HIV-1 Env gp120 Antibody VRC01
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 108 - protein, 131 aa, Homo sapiens
  MISC_FEATURE (1)..(131): VH Domain of Human Anti-HIV-1 gp41 Antibody 10E8
EVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSS

SEQ ID NO: 109 - protein, 109 aa, Homo sapiens
  MISC_FEATURE (1)..(109): VL Domain of Human Anti-HIV-1 gp41 Antibody 10E8
SYELTQETGVSVALGRTVTITCRGDSLRSHYASWYQKKPGQAPILLFYGKNNRPSGVPDRFSGSASGNRASLTISGAQAEDDAEYYCSSRDKSGSRLSVFGGGTKLTVL

SEQ ID NO: 110 - protein, 509 aa, Artificial Sequence
  First Polypeptide Chain of DART-1
DIVMTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 277 aa, Artificial Sequence
  Second Polypeptide Chain of DART-1
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSSASTKGKVAACKEKVAALKEKVAALKEKVAALKE

SEQ ID NO: 112 - protein, 227 aa, Artificial Sequence
  Third Polypeptide Chain of DART-1
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 113 - protein, 509 aa, Artificial Sequence
  First Polypeptide Chain of DART-A
DIVMTQSPDSLAVSLGERATINCKSSQTLLYSSNNRHSIAWYQQKPGQPPKLLIYWASMRLSGVPDRFSGSGSGTDFTLTISSLQAEDVAVYYCHQYSSHPPTFGQGTKVEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 277 aa, Artificial Sequence
  Second Polypeptide Chain of DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVESGGGLVKPGGSLRLSCAASGFTFSEYYMTWIRQAPGKGLEWVSYISKNGEYSKYSPSSNGRFTISRDNAKNSLYLQMNSLRAEDTAVYYCARADGLTYFSELLQYIFDLWGQGTLVTVSSASTKGKVAACKEKVAALKEKVAALKEKVAALKE

SEQ ID NO: 115 - protein, 502 aa, Artificial Sequence
  First Polypeptide Chain of DART-B
DIVMTQSPDSLAVSLGERATINCKSSQTLLYSSNNRHSIAWYQQKPGQPPKLLIYWASMRLSGVPDRFSGSGSGTDFTLTISSLQAEDVAVYYCHQYSSHPPTFGQGTKVEIKGGGSGGGGQVTLRESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 278 aa, Artificial Sequence
  Second Polypeptide Chain of DART-B
DIVMTQSPDSLAVSLGERATINCKASQSVDFDGDSFMNWYQQKPGQPPKLLIYTTSNLESGVPDRFSGSGSGTDFTLTISSLQAEDVAVYYCQQSNEDPYTFGQGTKLEIKGGGSGGGGQVQLVESGGGLVKPGGSLRLSCAASGFTFSEYYMTWIRQAPGKGLEWVSYISKNGEYSKYSPSSNGRFTISRDNAKNSLYLQMNSLRAEDTAVYYCARADGLTYFSELLQYIFDLWGQGTLVTVSSASTKGKVAACKEKVAALKEKVAALKEKVAALKE

SEQ ID NO: 117 - protein, 451 aa, Artificial Sequence
  Third Polypeptide Chain of TRIDENT-A
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 118 - protein, 213 aa, Artificial Sequence
  Fourth Polypeptide Chain of TRIDENT-A
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 453 aa, Artificial Sequence
  Third Polypeptide Chain of TRIDENT-B
QVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 120 - protein, 217 aa, Artificial Sequence
  Fourth Polypeptide Chain of TRIDENT-B
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 456 aa, Artificial Sequence
  Third Polypeptide Chain of TRIDENT-D
QVQLVESGGGLVKPGGSLRLSCAASGFTFSEYYMTWIRQAPGKGLEWVSYISKNGEYSKYSPSSNGRFTISRDNAKNSLYLQMNSLRAEDTAVYYCARADGLTYFSELLQYIFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 122 - protein, 220 aa, Artificial Sequence
  Fourth Polypeptide Chain of TRIDENT-D
DIVMTQSPDSLAVSLGERATINCKSSQTLLYSSNNRHSIAWYQQKPGQPPKLLIYWASMRLSGVPDRFSGSGSGTDFTLTISSLQAEDVAVYYCHQYSSHPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 101 aa, Homo sapiens
  MISC_FEATURE (1)..(101): VL Domain of Framework 1-3 Donor (IGKV4-1)
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 124 - protein, 10 aa, Homo sapiens
  MISC_FEATURE (1)..(10): VL Domain of Framework 4 Donor (IGKJ1-01)
FGQGTKVEIK

SEQ ID NO: 125 - protein, 98 aa, Homo sapiens
  MISC_FEATURE (1)..(98): VH Domain of Framework 1-3 Donor (IGVH3-11)
QVQLVESGGGLVKPGGSLRLSCAASGFTFSDYYMSWIRQAPGKGLEWVSYISSSSSYTNYADSVKGRFTISRDNAKNSLYLQMNSLRAEDTAVYYCAR

SEQ ID NO: 126 - protein, 11 aa, Homo sapiens
  MISC_FEATURE (1)..(11): VH Domain of Framework 4 Donor (IGHJ4-01)
WGQGTLVTVSS

SEQ ID NO: 127 - protein, 118 aa, Artificial Sequence
  VH Domain of Humanized Anti-Human CD16 Antibody hCD16-M1
EVQLVESGGGLVKPGGSLRLSCAASGFTFSNYGMSWVRQAPGKGLEWVATISGGGSYTFYPDSVKGRFTISRDNAKNSLYLQMNSLRTEDTALYYCVRQSARAPEPYWGQGTLVTVSS

SEQ ID NO: 128 - protein, 107 aa, Artificial Sequence
  VL Domain of Humanized Anti-Human CD16 Antibody hCD16-M1
DIQMTQSPSFLSASVGDRVTITCRASQNVGTHVAWYQQKPGKAPKSLLYSASYRYSGVPSRFSGSGSGTDFTLTISSLQSEDIATYYCQQYKSYPLTFGQGTKLEIK

SEQ ID NO: 129 - protein, 119 aa, Artificial Sequence
  VH Domain of Humanized Anti-Human CD16 Antibody CD16-M2 VH1
QVQLVQSGAEVKKPGASVKVSCKASGYTFTSSAMHWVRQAPGQGLEWMGYINHYNDGIKYNERFKGRVTITADKSTSTAYMELSSLRSEDTAVYYCATGYRYASWFASWGQGTLVTVSS

SEQ ID NO: 130 - protein, 119 aa, Artificial Sequence
  VL Domain of Humanized Anti-Human CD16 Antibody CD16-M2 VH2
QVQLVQSGAEVKKPGASVKVSCKASGYTFTSSAMHWVRQAPGQGLEWMGYINHYNDGIKYNERFKGRVTITADKSTSTAYMELSSLRSEDTAVYYCARGYRYASWFASWGQGTLVTVSS

SEQ ID NO: 131 - protein, 107 aa, Artificial Sequence
  VL Domain of Humanized Anti-Human CD16 Antibody hCD16-M2 VL1
EIVLTQSPATLSVSPGERATLSCRASQNIGTSIHWYQQKPDQSPKLLIKSVSESISGVPSRFSGSGSGTDFTLTINSLEAEDFATYYCQQSNSWPLTFGQGTKLEIK

(19) United States
(12) Patent Application Publication
(10) Pub. No.: US 2021/0246194 A1
(43) Pub. Date: Aug. 12, 2021

(54) Optimized gp41-Binding Molecules and Uses Thereof

(71) Applicants: MacroGenics, Inc., Rockville, MD (US); Duke University, Durham, NC (US)

(72) Inventors: Chia-Ying Kao Lam, San Jose, CA (US); Gundo Diedrich, North Potomac, MD (US); Jeffrey Lee Nordstrom, Olney, MD (US); Liqin Liu, Woodland Hills, CA (US); Leslie S. Johnson, Rockville, MD (US); Scott Koenig, Rockville, MD (US); Barton F. Haynes, Durham, NC (US); Guido Ferrari, Durham, NC (US)

(73) Assignees: MacroGenics, Inc., Rockville, MD (US); Duke University, Durham, NC (US)

(21) Appl. No.: 17/055,805

(22) PCT Filed: May 13, 2019
(86) PCT No.: PCT/US2019/032030

§ 371 Date: Nov. 16, 2020

Related U.S. Application Data
(60) Provisional application No. 62/673,462, filed on May 18, 2018.

(51) Int. Cl. C07K 16/10; C07K 16/28; A61K 39/42; A61K 45/06

(52) U.S. Cl. CPC C07K 16/1063; C07K 16/2809; C07K 16/2815; A61K 39/42; C07K 2317/567; C07K 2317/31; C07K 2317/626; C07K 2317/35; C07K 2317/52; A61K 45/06

(57) ABSTRACT

The present invention is directed to optimized HIV-1 gp41-Binding Molecules having reduced immunogenicity. More specifically, the invention relates to optimized gp41-Binding Molecules that comprise a gp41-binding Variable Light Chain (VL) Domain and/or a gp41-binding Variable Heavy Chain (VH) Domain that has/have been optimized to reduce the immunogenicity of such Domain(s) upon administration to a recipient subject. The invention particularly pertains to gp41-Binding Molecules that are multispecific gp41-Binding Molecules (including bispecific diabodies (including DART® diabodies), BiTE®s, bispecific antibodies, trivalent binding molecules (including TRIDENT™ molecules), etc.) that comprise: (i) such optimized gp41-binding Variable Domain(s) and (ii) a domain capable of binding to an epitope of a molecule present on the surface of an effector cell. The invention is also directed to pharmaceutical compositions that comprise any of such gp41-Binding Molecules, and to methods involving the use of any of such gp41-Binding Molecules in the treatment of HIV-1 infection.

### CROSS REFERENCE TO RELATED APPLICATIONS

This application claims priority to U.S. patent applications. Ser. No. 62/673,462 (filed on May 18, 2019; pending), which application is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0155PCT_ST25.txt, created on May 11, 2019, and having a size of 158,835 bytes), which file is incorporated herein in its entirety.

### FIELD OF THE INVENTION

The present invention is directed to optimized HIV-1 gp41-Binding Molecules having reduced immunogenicity. More specifically, the invention relates to optimized gp41-Binding Molecules that comprise a gp41-binding Variable Light Chain (VL) Domain and/or a gp41-binding Variable Heavy Chain (VH) Domain that has/have been optimized to reduce the immunogenicity of such Domain(s) upon administration to a recipient subject. The invention particularly pertains to gp41-Binding Molecules that are multispecific gp41-Binding Molecules (including bispecific diabodies (including DART® diabodies), BiTE®s, bispecific antibodies, trivalent binding molecules (including TRIDENT™ molecules), etc.) that comprise: (i) such optimized gp41-binding Variable Domain(s) and (ii) a domain capable of binding to an epitope of a molecule present on the surface of an effector cell. The invention is also directed to pharmaceutical compositions that comprise any of such gp41-Binding Molecules, and to methods involving the use of any of such gp41-Binding Molecules in the treatment of HIV-1 infection.

### BACKGROUND OF THE INVENTION

Highly Active Antiretroviral Therapy (HAART) has been effective in reducing the viral burden and ameliorating the effects of HIV type 1 (HIV-1) infection in infected individuals. However, HAART cannot eradicate HIV-1 infection, and it does not accelerate the elimination of infected cells. In infected individuals, HIV-1 persists in a latent state as integrated proviruses within resting memory CD4+ T cells that are not accessible to HAART. Thus, despite the administration of such therapy, the HIV-1 virus persists in the individual within a latent reservoir of HIV-1 infected cells that have evaded treatment. CD8+ T cells have a limited ability to eliminate the HIV-1 in such latently infected cells.

Thus, there is a need for therapeutic agents for treatment of HIV-1 infected individuals, particularly agents that target virus infected cells and have the potential to reduce the latent reservoir of HIV-1 infected cells and which are suitable for repeated administration.

The envelope glycoprotein (“Env”) of the HIV-1 virus is crucial to viral infectivity (via its ability to bind to the CD4 receptor) and for driving membrane fusion. The HIV-1 Env gene product consists of a trimeric complex of two subunits, gp120 and gp41. The Env protein is synthesized as a glycosylated gp160 precursor protein, which is folded into trimers and proteolytically cleaved to yield the mature gp120 and gp41 proteins. The cleaved Env is assembled, together with other viral components, for virion budding from the cell surface and is present on the surface of infected cells (Miranda, L., et al., 2002 “Cell Surface Expression Of The HIV-1 Envelope Glycoproteins Is Directed From Intracellular CTLA-4-Containing Regulated Secretory Granules.” Proc Natl Acad Sci USA. 99(12): 8031-8036). Thus, HIV Env, and particularly its gp41 subunit, is a highly specific viral target for therapeutic elimination of the persistent HIV infected reservoirs via antibody-mediated cell killing (Sloane, D., et al., 2015, “Targeting HIV Reservoir in Infected CD4 T Cells by Dual-Affinity Re-targeting Molecules (DARTs) that Bind HIV Envelope and Recruit Cytotoxic T Cells” PLOS Pathogens DOI:10.1371/journal.ppat.1005233.

However, despite all prior advances, a need still remains for optimized HIV gp41-binding molecules having enhanced anti-HIV activity and/or reduced immunogenicity. The present invention addresses this need and the need for improved therapeutics for HIV-1 treatment and prevention.

### SUMMARY OF THE INVENTION

The present invention is directed to optimized HIV-1 gp41-Binding Molecules having reduced immunogenicity. More specifically, the invention relates to optimized gp41-Binding Molecules that comprise a gp41-binding Variable Light Chain (VL) Domain and/or a gp41-binding Variable Heavy Chain (VH) Domain that has/have been optimized to reduce the immunogenicity of such Domain(s) upon administration to a recipient subject. The invention particularly pertains to gp41-Binding Molecules that are multispecific gp41-Binding Molecules (including bispecific diabodies (including DART® diabodies), BiTE®s, bispecific antibodies, trivalent binding molecules (including TRIDENT™ molecules), etc.) that comprise: (i) such optimized gp41-binding Variable Domain(s) and (ii) a domain capable of binding to an epitope of a molecule present on the surface of an effector cell. The invention is also directed to pharmaceutical compositions that comprise any of such gp41-Binding Molecules, and to methods involving the use of any of such gp41-Binding Molecules in the treatment of HIV-1 infection.

In detail, the invention provides a gp41-Binding Molecule comprising a Variable Light Chain (VL) Domain and a Variable Heavy Chain (VH) Domain, wherein the VL Domain comprises the amino acid sequence of SEQ ID NO:57 and/or the VH Domain comprises the amino acid sequence of SEQ ID NO:58.

The invention further relates to the embodiment of the above-indicated gp41-Binding Molecule, wherein the gp41-Binding Molecule comprises:
(a) the VL Domain that comprises the amino acid sequence of SEQ ID NO:57 and
(b) the VH Domain that comprises the amino acid sequence of SEQ ID NO:58.

The invention further relates to the embodiments of the above-indicated gp41-Binding Molecule, wherein the molecule is an antibody or comprises a gp41 epitope-binding portion thereof.

The invention further relates to the embodiment of the above-indicated gp41-Binding Molecules, wherein the molecule is:
(a) a bispecific antibody; or
(b) a diabody, the diabody being a covalently bonded complex that comprises two, three, four or five polypeptide chains; or
(c) a trivalent binding molecule, the trivalent binding molecule being a covalently bonded complex that comprises three, four, five, or more than five polypeptide chains.

The invention further relates to the embodiment of the above-indicated gp41-Binding Molecules, wherein the molecule is the diabody and comprises an Albumin-Binding Domain (ABD).

The invention further relates to the embodiment of the above-indicated gp41-Binding Molecules, wherein the molecule comprises an Fc Region. The invention further relates to the embodiment of such gp41-Binding Molecules, wherein the Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

The invention further relates to the embodiment of such gp41-Binding Molecules, wherein the modifications that reduce the affinity of the variant Fc Region for an FcγR comprise the substitution of L234A; L235A; or L234A and L235A, wherein the numbering is that of the EU index as in Kabat.

The invention further relates to the embodiment of such gp41-Binding Molecules, wherein the modifications that enhance the serum half-life of the variant Fc Region comprise the substitution of M252Y; M252Y and S254T; M252Y and T256E; M252Y, S254T and T256E; or K288D and H435K, wherein the numbering is that of the EU index as in Kabat.

The invention further relates to the embodiment of the above-indicated gp41-Binding Molecules, wherein the molecule is bispecific and comprises one epitope-binding site capable of immunospecific binding to an epitope of gp41 and one epitope-binding site capable of immunospecific binding to an epitope of a molecule present on the surface of an effector cell.

The invention further relates to the embodiment of the above-indicated gp41-Binding Molecules, wherein the molecule is bispecific and comprises two epitope-binding sites capable of immunospecific binding to an epitope of gp41 and two epitope-binding sites capable of immunospecific binding to an epitope of a molecule present on the surface of an effector cell.

The invention further relates to the embodiment of the above-indicated gp41-Binding Molecules, wherein the molecule is trispecific and comprises:
(a) one epitope-binding site capable of immunospecific binding to an epitope of gp41;
(b) one epitope-binding site capable of immunospecific binding to an epitope of a first molecule present on the surface of an effector cell; and
(c) one epitope-binding site capable of immunospecific binding to an epitope of a second molecule present on the surface of an effector cell.

The invention further relates to the embodiment of such gp41-Binding Molecules, wherein the first molecule present on the surface of an effector cell is CD3 and the second molecule present on the surface of an effector cell is CD8.

The invention further relates to the embodiment of the above-indicated gp41-Binding Molecules, wherein the molecule is trispecific and comprises:
(a) one epitope-binding site capable of immunospecific binding to a first epitope of gp41;
(b) one epitope-binding site capable of immunospecific binding to a second epitope of gp41 or an epitope of a different HIV-1 protein; and
(c) one epitope-binding site capable of immunospecific binding to an epitope a molecule present on the surface of an effector cell;
wherein the first and second epitopes of gp41 are different.

The invention further relates to the embodiment of the above-indicated gp41-Binding Molecules, wherein the molecule is capable of simultaneously binding to gp41 and the molecule present on the surface of an effector cell. The invention further relates to the embodiment of such gp41-Binding Molecules, wherein the molecule present on the surface of an effector cell is CD2, CD3, CD8, CD16, TCR, NKp46, or NKG2D.

The invention further relates to the embodiment of the above-indicated gp41-Binding Molecules, wherein the effector cell is a cytotoxic T-cell, or a Natural Killer (NK) cell.

The invention further relates to the embodiment of the above-indicated gp41-Binding Molecules, wherein the molecule mediates coordinated binding of a cell expressing gp41 and a cytotoxic T cell.

The invention further relates to the embodiment of such gp41-Binding Molecules, wherein the molecule comprises a first polypeptide chain, a second polypeptide chain and a third polypeptide chain, and wherein:
I) (a) the first polypeptide chain comprises SEQ ID NO:113;
   (b) the second polypeptide chain comprises SEQ ID NO:114; and
   (c) the third polypeptide chain comprises SEQ ID NO:112;
   or
II) (a) the first polypeptide chain comprises SEQ ID NO:115;
   (b) the second polypeptide chain comprises SEQ ID NO:116; and
   (c) the third polypeptide chain comprises SEQ ID NO:112.

The invention further relates to the embodiment of such gp41-Binding Molecules, wherein the molecule comprises a first polypeptide chain, a second polypeptide chain, a third polypeptide chain, and a fourth polypeptide chain and wherein:
(a) the first polypeptide chain comprises SEQ ID NO:113;
(b) the second polypeptide chain comprises SEQ ID NO:114;
(c) the third polypeptide chain comprises SEQ ID NO:117; and
(d) the fourth polypeptide chain comprises SEQ ID NO:118.

The invention further relates to the embodiment of such gp41-Binding Molecules, wherein the molecule comprises a first polypeptide chain, a second polypeptide chain, a third polypeptide chain, and a fourth polypeptide chain and wherein:
I) (a) the first polypeptide chain comprises SEQ ID NO:113;
   (b) the second polypeptide chain comprises SEQ ID NO:114;
   (c) the third polypeptide chain comprises SEQ ID NO:119; and
   (d) the fourth polypeptide chain comprises SEQ ID NO:120;
   or
II) (a) the first polypeptide chain comprises SEQ ID NO:115;
   (b) the second polypeptide chain comprises SEQ ID NO:116;
   (c) the third polypeptide chain comprises SEQ ID NO:119; and
   (d) the fourth polypeptide chain comprises SEQ ID NO:120.

The invention further relates a pharmaceutical composition that comprises an effective amount of the gp41-Binding Molecule of any of claims 1-22 and a pharmaceutically acceptable carrier.

The invention further relates to a method to treat or prevent HIV-1 infection in a subject in need thereof comprising administering to the subject a composition comprising any one of the above-indicated gp41-Binding Molecules or the above-indicated pharmaceutical composition in a therapeutically effective amount.

The invention further relates to the embodiment of such method to treat or prevent HIV-1 infection that further comprises administering a latency-activating agent (such as vorinostat, romidepsin, panobinostat, disulfiram, JQ1, bryostatin, PMA, inonomycin, or any combination thereof).

### BRIEF DESCRIPTION OF THE DRAWINGS

FIGS. 1A-1B provide a schematic of a representative covalently bonded diabody having two Epitope Binding Domains composed of two polypeptide chains, each having an E-coil or K-coil Heterodimer-Promoting Domain (alternative Heterodimer-Promoting Domains are provided below). A cysteine residue may be present in a linker (FIG. 1A) and/or in the Heterodimer-Promoting Domain (FIG. 1B). VL and VH Domains that recognize the same epitope are shown using the same shading or fill pattern. The wavy line (WWW) in this and all of the Figures providing schematic presentations of binding molecule domains represents one or more optional Heterodimer-Promoting Domains, that is/are preferably present.

FIG. 2 provides a schematic of a representative covalently bonded diabody molecule having two Epitope Binding Domains composed of two polypeptide chains, each having a CH2 and CH3 Domain, such that the associated chains form all or part of an Fc Region. VL and VH Domains that recognize the same epitope are shown using the same shading or fill pattern.

FIGS. 3A-3C provide schematics showing representative covalently bonded tetravalent diabodies having four Epitope Binding Domains composed of two pairs of polypeptide chains (i.e., four polypeptide chains in all). One polypeptide chain of each pair possesses a CH2 and CH3 Domain, such that the associated chains form all or part of an Fc Region. VL and VH Domains that recognize the same epitope are shown using the same shading or fill pattern. The two pairs of polypeptide chains may be same. In such embodiments, wherein the two pairs of polypeptide chains are the same and the VL and VH Domains recognize different epitopes (as shown in FIGS. 3A-3B), the resulting molecule possesses four Epitope Binding Domains and is bispecific and bivalent with respect to each bound epitope. In such embodiments, wherein the VL and VH Domains recognize the same epitope (e.g., the same VL Domain CDRs and the same VH Domain CDRs are used on both chains) the resulting molecule possesses four Epitope Binding Domains and is monospecific and tetravalent with respect to a single epitope. Alternatively, the two pairs of polypeptides may be different. In such embodiments, wherein the two pairs of polypeptide chains are different and the VL and VH Domains of each pair of polypeptides recognize different epitopes (as shown by the different shading and patterns in FIG. 3C), the resulting molecule possesses four Epitope Binding Domains and is tetraspecific and monovalent with respect to each bound epitope. FIG. 3A shows an Fc Region-containing diabody which contains a peptide Heterodimer-Promoting Domain comprising a cysteine residue. FIG. 3B shows an Fc Region-containing diabody, which contains E-coil and K-coil Heterodimer-Promoting Domains comprising a cysteine residue and a linker (with an optional cysteine residue). FIG. 3C, shows an Fc-Region-Containing diabody, which contains antibody CH1 and CL domains. As provided below, the VL/VH binding sites formed by the association of the polypeptide chains may be the same or different so as to permit tetravalent binding that is monospecific, bispecific, trispecific or tetraspecific.

FIGS. 4A-4B provide schematics of a representative covalently bonded diabody molecule having two Epitope Binding Domains composed of three polypeptide chains. Two of the polypeptide chains possess a CH2 and CH3 Domain, such that the associated chains form all or part of an Fc Region. The polypeptide chains comprising the VL and VH Domain further comprise a Heterodimer-Promoting Domain. VL and VH Domains that recognize the same epitope are shown using the same shading or fill pattern.

FIG. 5 provides schematics of a representative covalently bonded Binding Molecule having four Epitope Binding Domains composed of five polypeptide chains. Two of the polypeptide chains possess a CH2 and CH3 Domain, such that the associated chains form an Fc Region that comprises all or part of an Fc Region. The polypeptide chains comprising the linked VL and VH Domains further comprise a Heterodimer-Promoting Domain. VL and VH Domains that recognize the same epitope are shown using the same shading or fill pattern. As provided below, the VL/VH binding sites formed by the association of the polypeptide chains may be the same or different so as to permit tetravalent binding that is monospecific, bispecific, trispecific or tetraspecific.

FIGS. 6A-6F provide schematics of representative Fc Region-containing trivalent binding molecules having three Epitope Binding Domains. FIG. 6A schematically the domains of trivalent binding molecules comprising two diabody-type binding domains and a Fab-type binding domain having different domain orientations in which the diabody-type binding domains are N-terminal to an Fc Region. FIG. 6B illustrates schematically the domains of trivalent binding molecules comprising two diabody-type binding domains and a Fab-type binding domain having different domain orientations in which the diabody-type binding domains are C-terminal to an Fc Region. The molecules in FIGS. 6A-6B comprise four chains. FIGS. 6C and 6D, respectively, illustrate schematically the domains of trivalent binding molecules comprising two diabody-type binding domains N-terminal to an Fc Region, and a Fab-type binding domain in which the light chain and heavy chain are linked via a polypeptide spacer, or an scFv-type binding domain. The trivalent binding molecules in FIGS. 6E and 6F, respectively, illustrate schematically the domains of trivalent binding molecules comprising two diabody-type binding domains C-terminal to an Fc Region, and a Fab-type binding domain in which the light chain and heavy chain are linked via a polypeptide spacer, or an scFv-type binding domain. The trivalent binding molecules in FIGS. 6C-6F comprise three chains. VL and VH Domains that recognize the same epitope are shown using the same shading or fill pattern.

FIGS. 7A-7B show the alignments of the amino acid sequences of 7B2, germlined 7B2 (7B2GL) and the indicated human germlines. The differences from 7B2 are shaded, the framework (FW) and CDR regions are indicated and the Kabat numbering is provided. The VL Domain is presented in FIG. 7A, and the VL Domain is presented in FIG. 7B.

FIGS. 8A-8B show the sensograms of recombinant HIV-1 JRFL gp140 (100, 50, 25 nM) binding to immobilized 7B2 IgG (FIG. 8A) and 7B2GL IgG (FIG. 8B) as measured by Attana Cell A200 QCM.

FIG. 9 shows the ability of DART-A (comprising 7B2GL VL and VH Domains optimized to minimize immunogenicity), and DART-1 (comprising the parental 7B2 VL and VH Domains) to bind to the surface of gp140-expressing HEK 293/D371 cells.

FIG. 10 shows the ability of DART-A (comprising 7BGL VL and VH Domains optimized to minimize immunogenicity), and DART-1 (comprising the parental 7B2 VL and VH Domains) to activate T cells as measured in a Jurkat T cell reporter assay.

FIG. 11 shows the ability of DART-A (comprising 7BGL VL and VH Domains optimized to minimize immunogenicity), and DART-1 (comprising the parental 7B2 VL and VH Domains) to mediate T cell redirected killing of HIV gp41-expressing target cells in a CTL assay.

### DETAILED DESCRIPTION OF THE INVENTION

The present invention is directed to optimized HIV-1 gp41-Binding Molecules having reduced immunogenicity. More specifically, the invention relates to optimized gp41-Binding Molecules that comprise a gp41-binding Variable Light Chain (VL) Domain and/or a gp41-binding Variable Heavy Chain (VH) Domain that has/have been optimized to reduce the immunogenicity of such Domain(s) upon administration to a recipient subject. The invention particularly pertains to gp41-Binding Molecules that are multispecific gp41-Binding Molecules (including bispecific diabodies (including DART® diabodies), BiTE®s, bispecific antibodies, trivalent binding molecules (including TRIDENT™ molecules), etc.) that comprise: (i) such optimized gp41-binding Variable Domain(s) and (ii) a domain capable of binding to an epitope of a molecule present on the surface of an effector cell. The invention is also directed to pharmaceutical compositions that comprise any of such gp41-Binding Molecules, and to methods involving the use of any of such gp41-Binding Molecules in the treatment of HIV-1 infection.

### I. Antibodies and Other Binding Molecules

A. Antibodies

The gp41-Binding Molecules of the present invention may be antibodies, or may be derivable from gp41-binding antibodies (e.g., by fragmentation, cleavage, etc. of antibody polypeptides), or obtained from the use of the amino acid sequence of one or more of the polypeptide chains of antibody molecules, or expressed by polynucleotides that encode such polypeptides, or obtained from the nucleotide sequences of such polynucleotides.

Antibodies are immunoglobulin molecules capable of specific binding to a particular domain or moiety or conformation (an “epitope”) of a molecule, such as a carbohydrate, polynucleotide, lipid, polypeptide, etc. An epitope-containing molecule may have immunogenic activity, such that it elicits an antibody production response in an animal; such molecules are termed “antigens.” As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab portions, F(ab′) portions, disulfide-linked bispecific Fvs (sdFv), intrabodies, and Epitope Binding Domains of any of the above. Such Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass, or species (bovine, equine, feline, canine, rodent, primate (e.g., including monkey such as, a cynomolgus monkey, human, etc.).

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also portions thereof (such as Fab, Fab′, F(ab′)₂, (Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the portions etc. described above under the definition of “antibody.”

Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freund's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody as detailed below.

Antibodies and the Binding Molecules of the present invention bind epitopes via their Binding Domains in an “immunospecific” manner. As used herein, a molecule is said to bind an epitope of another molecule in an immunospecific manner (or “immunospecifically”) if it binds or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind a second target. As such, “immunospecific binding” does not necessarily require (although it can include) exclusive binding. Generally, but not necessarily, reference to binding means “immunospecific” binding. Natural antibodies are capable of binding to only one epitope species (i.e., they are “monospecific”), although they can immunospecifically bind multiple copies of that species (i.e., exhibiting “bivalency” or “multivalency”). Two molecules are said to be capable of binding one another in a “physiospecific” manner, if such binding exhibits the specificity with which receptors bind their respective ligands.

The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

1. General Structural Attributes of Antibodies

The basic structural unit of naturally occurring immunoglobulins (e.g., IgG) is a tetramer composed of two shorter “Light Chains” complexed with two longer “Heavy Chains” and is usually expressed as a glycoprotein of about 150,000 Da. Each chain is composed of an amino-terminal (“N-terminal”) portion that comprises a “Variable Domain” and a carboxy-terminal (“C-terminal”) portion that comprises at least one “Constant Domain.” An IgG Light Chain is composed of a single “Light Chain Variable Domain” (“VL”) and a single “Light Chain Constant Domain” (“CL”). Thus, the structure of the light chains of an IgG molecule is n-VL-CL-c (where n and c represent, respectively, the N-terminus and the C-terminus of the polypeptide). An IgG Heavy Chain is composed of a single “Heavy Chain Variable Domain” (“VH”), three “Heavy Chain Constant Domains” (“CH1,” “CH2” and “CH3”), and a “Hinge” Region (“H”), located between the CH1 and CH2 Domains. Thus, the structure of an IgG heavy chain is n-VH-CH1-H-CH2-CH3-c (where n and c represent, respectively, the N-terminus and the C-terminus of the polypeptide). The ability of an intact, unmodified antibody (e.g., an IgG antibody) to bind an epitope of an antigen depends upon the presence and sequences of the Variable Domains. Unless specifically noted, the order of domains of the protein molecules described herein is in the “N-terminal to C-terminal” direction.

(a) Constant Domains

(i) Light Chain Constant Domain

A preferred CL Domain is a human IgG CL Kappa Domain. The amino acid sequence of an exemplary human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:2):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

(ii) Heavy Chain CH1 Domains

The CH1 Domains of the two Heavy Chains of an antibody complex with the antibody's Light Chain's “CL” constant region, and are attached to the Heavy Chain's CH2 Domains via an intervening Hinge Domain.

An exemplary CH1 Domain is a human IgG1 CH1 Domain. The amino acid sequence of an exemplary human IgG1 CH1 Domain is (SEO ID NO:3):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH1 Domain. The amino acid sequence of an exemplary human IgG2 CH1 Domain is (SEQ ID NO:4):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG3 CH1 Domain. The amino acid sequence of an exemplary human IgG3 CH1 Domain is (SEQ ID NO:5):

ASTKGPSVFP LAPCSRSTSG GTAALGCLVK DYFPEPVTVS
WNSGALTSGV HTFPAVLQSS GLYSLSSVVT VPSSSLGTQT
YTCNVNHKPS NTKVDKRV

An exemplary CH1 Domain is a human IgG4 CH1 Domain. The amino acid sequence of an exemplary human IgG4 CH1 Domain is (SEQ ID NO:6):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

(b) Heavy Chain Hinge Regions

One exemplary Hinge Domain is a human IgG1 Hinge Domain. The amino acid sequence of an exemplary human IgG1 Hinge Domain is (SEQ ID NO:7):

EPKSCDKTHTCPPCP.

Another exemplary Hinge Domain is a human IgG2 Hinge Domain. The amino acid sequence of an exemplary human IgG2 Hinge Domain is (SEQ ID NO:8): ERKCCVECPPCP.

Another exemplary Hinge Domain is a human IgG3 Hinge Domain. The amino acid sequence of an exemplary human IgG3 Hinge Domain is (SEQ ID NO:9):

ELKTPLGDTT HTCPRCPEPK SCDTPPPCPR CPEPKSCDTP
PPCPRCPEPK SCDTPPPCPR CP.

Another exemplary Hinge Domain is a human IgG4 Hinge Domain. The amino acid sequence of an exemplary human IgG4 Hinge Domain is (SEQ ID NO:10): ESKYGPPCPSCP. As described herein, an IgG4 Hinge Domain may comprise a stabilizing mutation such as the S228P substitution. The amino acid sequence of an exemplary S228P-stabilized human IgG4 Hinge Domain is (SEQ ID NO:11): ESKYGPPCPPCP.

(c) Heavy Chain CH2 and CH3 Domains

The CH2 and CH3 Domains of the two heavy chains interact to form the “Fc Domain” of IgG antibodies that is recognized by cellular Fc Receptors, including, but not limited to, Fc gamma Receptors (FcγRs). As used herein, the term “Fc Region” is used to define a C-terminal region of an IgG heavy chain. A portion of an Fc Region (including a portion that encompasses an entire Fc Region) is referred to herein as an “Fc Domain.” An Fc Region is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of an exemplary human IgG1 is (SEQ ID NO:12):

```
231 240 250 260 270
APELLGGPSV FLFPPKPKDT LMISRTPEVT CVVVDVSHED
                  280 290 300 310
PEVKFNWYVD GVEVHNAKTK PREEQYNSTY RVVSVLTVLH
                  320 330 340 350
QDWLNGKEYK CKVSNKALPA PIEKTISKAK GQPREPQVYT
                  360 370 380 390
LPPSREEMTK NQVSLTCLVK GFYPSDIAVE WESNGQPENN
                  400 410 420 430
YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is lysine (K) or is absent.

The amino acid sequence of the CH2-CH3 Domain of an exemplary human IgG2 is (SEQ ID NO:13):

```
231 240 250 260 270
APPVA-GPSV FLFPPKPKDT LMISRTPEVT CVVVDVSHED
                  280 290 300 310
PEVQFNWYVD GVEVHNAKTK PREEQFNSTF RVVSVLTVVH
                  320 330 340 350
QDWLNGKEYK CKVSNKGLPA PIEKTISKTK GQPREPQVYT
                  360 370 380 390
LPPSREEMTK NQVSLTCLVK GFYPSDISVE WESNGQPENN
                  400 410 420 430
YKTTPPMLDS DGSFFLYSKL TVDKSRWQQG NVFSCSVMHE
                  440 447
ALHNHYTQKS LSLSPGX
```

as numbered by the EU index as set forth in Kabat, wherein X is lysine (K) or is absent.

The amino acid sequence of the CH2-CH3 Domain of an exemplary human IgG3 is (SEQ ID NO:14):

```
231 240 250 260 270
APELLGGPSV FLFPPKPKDT LMISRTPEVT CVVVDVSHED
                  280 290 300 310
PEVQFKWYVD GVEVHNAKTK PREEQYNSTF RVVSVLTVLH
                  320 330 340 350
QDWLNGKEYK CKVSNKALPA PIEKTISKTK GQPREPQVYT
                  360 370 380 390
LPPSREEMTK NQVSLTCLVK GFYPSDIAVE WESSGQPENN
                  400 410 420 430
YNTTPPMLDS DGSFFLYSKL TVDKSRWQQG NIFSCSVMHE
                  440 447
ALHNRFTQKS LSLSPGX
```

as numbered by the EU index as set forth in Kabat, wherein X is lysine (K) or is absent.

The amino acid sequence of the CH2-CH3 Domain of an exemplary human IgG4 is (SEQ ID NO:15):

```
231 240 250 260 270
APEFLGGPSV FLFPPKPKDT LMISRTPEVT CVVVDVSQED
                  280 290 300 310
PEVQFNWYVD GVEVHNAKTK PREEQFNSTY RVVSVLTVLH
                  320 330 340 350
QDWLNGKEYK CKVSNKGLPS SIEKTISKAK GQPREPQVYT
                  360 370 380 390
LPPSQEEMTK NQVSLTCLVK GFYPSDIAVE WESNGQPENN
                  400 410 420 430
YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by reference. The term “EU index as in Kabat” refers to the numbering of the constant domains of human IgG1 EU antibody. Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: Glm (1, 2, 3, 17) or Glm (a, x, f, z), G2m (23) or G2m (n), G3m (5, 6, 10, 11, 13, 14, 15, 16, 21, 24, 26, 27, 28) or G3m (bl, c3, b3, b0, b3, b4, s, t, g1, c5, u, v, g5) (Lefranc et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the gp41-Binding Molecules of the invention. Specifically encompassed by the instant invention are gp41-Binding Molecules lacking the C-terminal residue of the CH3 Domain. Also specifically encompassed by the instant invention are such constructs comprising the C-terminal lysine residue of the CH3 Domain.

(d) Variable Domains

The Variable Domains of an IgG molecule consist of three “complementarity determining regions” (“CDRs”), which contain the amino acid residues of the antibody that will be in contact with the epitope, as well as intervening non-CDR segments, referred to as “framework regions” (“FRs”), which, in general maintain the structure and determine the positioning of the CDR loops so as to permit such contacting (although certain framework residues may also contact the epitope). Thus, the VL and VH Domains have the structure n-FR1-CDR1-FR2-CDR2-FR3-CDR3-FR4-c. The amino acid sequences of the CDRs determine whether an antibody will be able to bind to a particular epitope. Interaction of an antibody light chain with an antibody heavy chain and, in particular, interaction of their VL and VH Domains, forms an Epitope Binding Domain of the antibody.

Amino acids from the Variable Domains of the mature heavy and light chains of immunoglobulins are also designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical Structures For The Hypervariable Regions Of Immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain.

Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe®, etc.), or is another type of protein.

The term “Epitope Binding Domain” (for example, a gp41-Binding Domain) denotes a portion of a binding molecule (or a polypeptide having the amino acid sequence of such a portion) that contributes to the ability of the binding molecule to immunospecifically bind to an epitope (for example, an epitope of gp41). An Epitope Binding Domain may contain a VL or VH Domain of an antibody, or any 1, 2, 3, 4, or 5 of the CDR Domains of an antibody, or may contain all 6 of the CDR Domains of an antibody and, although capable of immunospecifically binding such epitope, may exhibit an immunospecificity, affinity or selectivity towards such epitope that differs from that of such antibody. An Epitope Binding Domain may contain only part of a CDR, namely the subset of CDR residues required for binding, termed the SDRs (Kim, J. H. et al. (2012) “Humanization By CDR Grafting And Specificity-Determining Residue Grafting,” Methods Mol. Biol. 907:237-245; Kim, K. S. et al. (2010) “Construction Of A Humanized Antibody To Hepatitis B Surface Antigen By Specificity-Determining Residues (SDR)-Grafting And De-Immunization,” Biochem. Biophys. Res. Commun. 396(2):231-237; Kashmiri, S. V. et al. (2005) “SDR Grafting—A New Approach To Antibody Humanization,” Methods 36(1):25-34; Gonzales, N. R. et al. (2004) “SDR Grafting Of A Murine Antibody Using Multiple Human Germline Templates To Minimize Its Immunogenicity,” Mol. Immunol. 41:863-872). Preferably, however, an Epitope Binding Domain will contain all 6 of the CDR Domains of such antibody. An Epitope Binding Domain may be a single polypeptide chain (e.g., an scFv), or may comprise two or more polypeptide chains, which may each have an amino terminus and a carboxy terminus (e.g., a diabody, a Fab portion, an Fab₂ portion, etc.), and which may be covalently bonded to one another via a disulfide bond.

2. Humanization of Antibodies

The invention also particularly encompasses Binding Molecules that comprise a VL or VH Domain of an antibody, and preferably both a VL and a VH Domain of an antibody. Preferably, such antibody is a humanized antibody. Monoclonal antibodies are typically prepared in non-human species, such as mouse or rabbit. The Variable and/or Constant Domains of such antibodies may be recognized as immunogens, thus provoking an immune response against them. Such molecules may however be “humanized” by introducing one or more amino acid substitutions in order to render such antibodies more like antibodies produced by humans. thereby reducing or eliminating their immunogenicity. The term “humanized” antibody refers to a chimeric molecule, generally prepared using recombinant techniques, having an Epitope Binding Domain of an immunoglobulin from a non-human species and a remaining immunoglobulin structure of the molecule that is based upon the structure and/or sequence of a human immunoglobulin. The polynucleotide sequence of the variable domains of such antibodies may be used for genetic manipulation to generate such derivatives and to improve the affinity, or other characteristics of such antibodies. Application of this approach to various antibodies has been reported by LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224; Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, and/or six) which differ in sequence relative to the original antibody.

The general principle in humanizing an antibody involves retaining the basic sequence of the Epitope Binding Domain of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

A number of humanized antibody molecules comprising an Epitope Binding Domain derived from a non-human immunoglobulin have been described, including chimeric antibodies having rodent or modified rodent Variable Domain and their associated complementarity determining regions (CDRs) fused to human constant domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse/Human Chimeric Monoclonal Antibody (17-1A) To A Colon Cancer Tumor Associated Antigen,” J. Immunol. 138:4534-4538, and Brown et al. (1987) “Tumor-Specific Genetically Engineered Murine/Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692. The invention particularly encompasses binding molecules (including antibodies and diabodies) that comprise a VL and/or VH Domain of a “humanized” antibody.

Notwithstanding such successes, the production of stable, functional heterodimeric, non-monospecific diabodies optimized for therapeutic use can be further improved by the careful consideration and placement of the domains employed in the polypeptide chains. The present invention is thus directed to the provision of specific polypeptides that are particularly designed to form, via covalent bonding, stable and therapeutically useful heterodimeric diabodies and heterodimeric Fc diabodies that are capable of simultaneously binding gp41 and a molecule present on the surface of an immune effector cell.

B. Bispecific Antibodies

As indicated above, natural antibodies are capable of binding only one epitope species (i.e., they are mono-specific), although they can bind multiple copies of that species (i.e., exhibiting bi-valency or multi-valency). The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two Epitope Binding Domains of a natural antibody, such as an IgG.

The functionality of antibodies can be enhanced by generating multispecific antibody-based molecules that can simultaneously bind two separate and distinct antigens (or different epitopes of the same antigen) and/or by generating antibody-based molecules having higher valency (i.e., more than two Binding Domains) for the same epitope and/or antigen.

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565). Most of such approaches use linker peptides to fuse a further binding domain (e.g. an scFv, VL, VH, etc.) to, or within the antibody core (IgA, IgD, IgE, IgG or IgM), or to fuse multiple antibody binding portions to one another (e.g. two Fab portions or scFv). Alternative formats use linker peptides to fuse a binding protein (e.g., an scFv, VL, VH, etc.) to a dimerization domain such as the CH2-CH3 Domain or alternative polypeptides (WO 2005/070966, WO 2006/107786A WO 2006/107617A, WO 2007/046893). Typically, such approaches involve compromises and trade-offs. For example, PCT Publication Nos. WO 2013/174873, WO 2011/133886 and WO 2010/136172 disclose that the use of linkers may cause problems in therapeutic settings, and teaches a trispecific antibody in which the CL and CH1 Domains are switched from their respective natural positions and the VL and VH Domains have been diversified (WO 2008/027236; WO 2010/108127) to allow them to bind to more than one antigen. Thus, the molecules disclosed in these documents trade binding specificity for the ability to bind additional antigen species. PCT Publication Nos. WO 2013/163427 and WO 2013/119903 disclose modifying the CH2 Domain to contain a fusion protein adduct comprising a binding domain. The document notes that the CH2 Domain likely plays only a minimal role in mediating effector function. PCT Publication Nos. WO 2010/028797, WO2010028796 and WO 2010/028795 disclose recombinant antibodies whose Fc Regions have been replaced with additional VL and VH Domains, so as to form trivalent binding molecules. PCT Publication Nos. WO 2003/025018 and WO2003012069 disclose recombinant diabodies whose individual chains contain scFv domains. PCT Publication Nos. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publication Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional Binding Domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another). Thus, the molecules disclosed in these documents trade native antibody structure for the ability to bind additional antigen species.

### II. Bispecific Diabodies

The art has additionally noted the capability of producing diabodies that differ from natural antibodies in being capable of binding two or more different epitope species (i.e., exhibiting bispecificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2): 1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T cell Engaging Antibodies For Cancer Therapy,” Cancer Res. 69(12):4941-4944).

The design of a diabody is based on the structure of the single-chain Variable Domain portion (scFv), in which Light and Heavy Chain Variable Domains are linked to one another using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The provision of non-monospecific “diabodies” provides a significant advantage over antibodies: the capacity to co-ligate and co-localize cells that express different epitopes. Bispecific diabodies thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their bivalency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221-1225).

The ability to produce bispecific diabodies has led to their use (in “trans”) to co-ligate two cells together, for example, by co-ligating receptors that are present on the surface of different cells (e.g., cross-linking cytotoxic T-cells to target cells, such as cancer cells or pathogen-infected cells, that express a disease antigen) (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305; Marvin et al. (2005) “Recombinant Approaches To IgG-Like Bispecific Antibodies,” Acta Pharmacol. Sin. 26:649-658; Sloan et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog 11(11): e1005233. doi:10.1371/journal.ppat.1005233)). Alternatively (or additionally), bispecific (or multispecific) diabodies can be used (in “cis”) to co-ligate molecules, such as receptors, etc., that are present on the surface of the same cell. Co-ligation of different cells and/or receptors is useful to modulate effector functions and/or immune cell signaling. Multispecific molecules (e.g., bispecific diabodies) comprising Epitope Binding Domains may be directed to a surface determinant of any immune cell such as CD2, CD3, CD8, CD16, TCR, NKG2D, etc., which are expressed on T lymphocytes, Natural Killer (NK) cells, Antigen-Presenting Cells or other mononuclear cells, or to a surface determinant of a B cell, such as CD19, CD20, CD22, CD30, CD37, CD40, and CD74 (Moore, P. A. et al. (2011) “Application Of Dual Affinity Retargeting Molecules To Achieve Optimal Redirected T cell Killing Of B-Cell Lymphoma,” Blood 117(17):4542-4551; Cheson, B. D. et al. (2008) “Monoclonal Antibody Therapy For B-Cell Non Hodgkin's Lymphoma,” N. Engl. J. Med. 359(6):613-626; Castillo, J. et al. (2008) “Newer Monoclonal Antibodies For Hematological Malignancies,” Exp. Hematol. 36(7):755-768). In particular, Epitope Binding Domains directed to a cell surface receptor that is present on immune effector cells, are useful in the generation of multispecific binding molecules capable of mediating redirected cell killing.

In many studies, diabody binding to effector cell determinants, e.g., Fcγ receptors (FcγR), was also found to activate the effector cell (Holliger et al. (1996) “Specific Killing Of Lymphoma Cells By Cytotoxic T cells Mediated By A Bispecific Diabody,” Protein Eng. 9:299-305; Holliger et al. (1999) “Carcinoembryonic Antigen (CEA)-Specific T cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen-bound antibody to an effector cell via an Fc Domain—FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of a tumor cell but leads to effective tumor killing (see e.g., Cao et al. (2003) “Bispecific Antibody Conjugates In Therapeutics,” Adv. Drug. Deliv. Rev. 55:171-197).

However, the advantages of the above-described bispecific diabodies come at a salient cost. The formation of such non-mono-specific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i. e., two polypeptide species) must be provided in order to form a non-mono-specific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional single polypeptide chain monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-mono-specific diabodies, termed DART® diabodies, see, e.g., Liu. L et al. (2017) “MGD011, A CD19×CD3 Dual-Affinity Retargeting Bi-specific Molecule Incorporating Extended Circulating Half-life for the Treatment of B-Cell Malignancies,” Clin Cancer Res. 23(6):1506-1518; Tsai, P. et al. (2016) “CD19×CD3 DART Protein Mediates Human B-Cell Depletion In Vivo In Humanized BLT Mice,” Mol. Ther. Oncolytics 3:15024. doi: 10.1038/mto.2015.24; Chen, X. et al. (2016) “Mechanistic Projection of First-in-Human Dose for Bispecific Immunomodulatory P-Cadherin LP-DART: An Integrated PK/PD Modeling Approach,” Clin. Pharmacol. Ther. 100(3):232-241; Sloan, D. D. et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog. 11(11):e1005233. doi: 10.1371/journal.ppat.1005233; Al Hussaini, M. et al. (2015) “Targeting CD123 In AML Using A T-Cell Directed Dual-Affinity Re-Targeting (DART®) Platform,” Blood pii: blood-2014-05-575704; Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIB (CD32B) Inhibitory Function With A Novel Bispecific Antibody Scaffold,” Arthritis Rheum. 62(7):1933-1943; Moore, P. A. et al. (2011) “Application Of Dual Affinity Retargeting Molecules To Achieve Optimal Redirected T cell Killing Of B-Cell Lymphoma,” Blood 117(17):4542-4551; U.S. Pat. Nos. 9,932,400; 9,908,938; 9,889,197; 9,884,921; 9,822,181; 9,296,816; 9,284,375; 8,795,667; 8,187,593; 8,669,349; 8,784,808; 8,795,667; 8,802,091; 8,802,093; 8,946,387 and 8,968,730, and US Patent Publication Nos. 2009/0060910; 2010/0174053; 2011/0081347; 2011/0097323; 2011/0117089; 2012/0034221; 2012/0294796; 2013/0149236; 2013/0295121; 2014/0017237; 2014/0099318; 2014/0255407; 2015/0175697; 2016/0017038; 2016/0159908; 2016/0159908; 2016/0194396; 2016/0194396; 2016/0200827; 2016/0200827; 2016/0222105; 2016/0222105; 2016/0333111; 2016/0355586; 2016/0355586; 2017/0157251; 2017/0157251; 2017/0198037; 2017/0198037; 2017/0198045; 2017/0204176; 2017/0204176; 2017/0247452; and 2018/0094072; European Patent Documents No. EP 1868650; EP 2158221; EP 2247304; EP 2252631; EP 2282770; EP 2328934; EP 2376109; EP 2542256; EP 2601216; EP 2714079; EP 2714733; EP 2786762; EP 2839842; EP 2840091; and PCT Publication Nos. WO 2006/113665; WO 2008/157379; WO 2010/027797; WO 2010/033279; WO 2010/080538; WO 2011/109400; WO 2012/018687; WO 2012/162067; WO 2012/162068; WO 2014/159940; WO 2015/021089; WO 2015/026892; and WO 2015/026894). Such diabodies comprise two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody's binding characteristics.

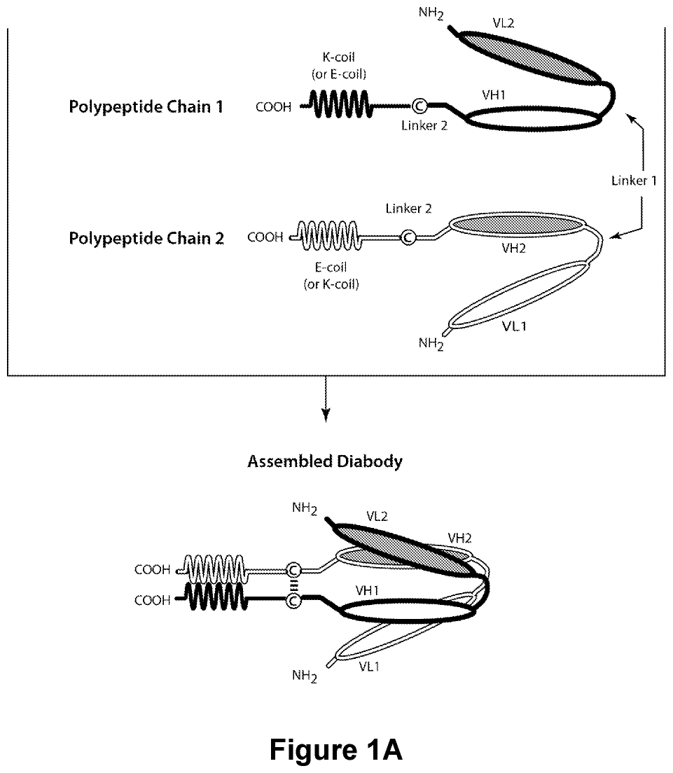

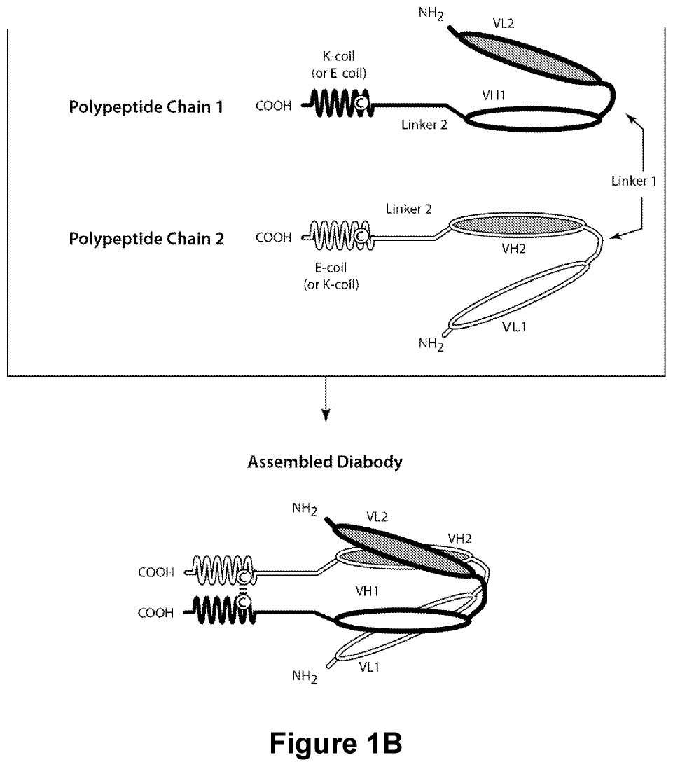

The simplest DART® diabody comprises two polypeptide chains each comprising three Domains (FIGS. 1A-1B). The first polypeptide chain comprises: (i) a Domain that comprises a binding region of a light chain variable Domain of the a first immunoglobulin (VL1), (ii) a second Domain that comprises a binding region of a heavy chain variable Domain of a second immunoglobulin (VH2), and (iii) a third Domain that serves to promote heterodimerization (a “Heterodimer-Promoting Domain”) with the second polypeptide chain and to covalently bond the first polypeptide to the second polypeptide chain of the diabody. The second polypeptide chain contains a complementary first Domain (a VL2 Domain), a complementary second Domain (a VH1 Domain) and a third Domain that complexes with the third Domain of the first polypeptide chain in order to promote heterodimerization (a “Heterodimer-Promoting Domain”) and covalent bonding with the first polypeptide chain. Such molecules are stable, potent and have the ability to simultaneously bind two or more antigens. In one embodiment, the third Domains of the first and second polypeptide chains each contain a cysteine (“©”) residue, which serves to bind the polypeptides together via a disulfide bond.

The third Domain of one or both of the polypeptide chains may additionally possess the sequence of a CH2-CH3 Domain, such that complexing of the diabody polypeptides forms an Fc Domain that is capable of binding to the Fc receptor of cells (such as B lymphocytes, dendritic cells, natural killer cells, macrophages, neutrophils, eosinophils, basophils and mast cells). Many variations of such molecules have been described (see, e.g., United States Patent Publication Nos. 2013-0295121; 2010-0174053; 2007-0004909; 2009-0060910; European Patent Publication No. EP 2714079; EP 2601216; EP 2376109; EP 2158221 and PCT Publication Nos. WO 2012/162068; WO 2012/018687; WO 2010/080538; WO 2006/113665) and are provided herein. Many variations of such molecules have been described (see, e.g., United States Patent Publication Nos. 2015/0175697; 2014/0255407; 2014/0099318; 2013/0295121; 2010/0174053; 2009/0060910; 2007-0004909; European Patent Publication Nos. EP 2714079; EP 2601216; EP 2376109; EP 2158221; EP 1868650; and PCT Publication Nos. WO 2012/162068; WO 2012/018687; WO 2010/080538; WO 2006/113665), and are provided herein.

Alternative constructs are known in the art for applications where a bispecific or tetravalent molecule is desirable but an Fc is not required including, but not limited to, Bispecific T cell Engager molecules, also referred to as “BITE® antibodies” (see, e.g., PCT Publication Nos: WO 1993/11161; and WO 2004/106381) and tetravalent tandem antibodies, also referred to as “TandAbs®” (see, e.g. United States Patent Publication No: 2011-0206672; European Patent Publication No. EP 2371866, and; PCT Publication Nos. WO 1999/057150, WO 2003/025018, and WO 2013/013700). BiTE®s are formed from a single polypeptide chain comprising tandem linked scFvs, while TandAb®s are formed by the homo-dimerization of two identical polypeptide chains, each possessing a VH1, VL2, VH2, and VL2 Domain.

### III. Embodiments of the Invention

The present invention is directed to “Epitope-Binding Molecules” that comprise “Epitope-Binding Domains,” such as a CDR_{L}1 Domain, CDR_{L}2 Domain, CDR_{L}3 Domain, CDR_{H}1 Domain, CDR_{H}2 Domain, or CDR_{H}3 Domain, or any combination or sub-combination thereof sufficient to form an “Epitope-Binding Site” that permits the molecule to bind to an epitope. For example, a “gp41-Epitope-Binding Molecule” comprises “Epitope-Binding Domains,” such as a CDR_{L}1 Domain, CDR_{L}2 Domain, CDR_{L}3 Domain, CDR_{H}1 Domain, CDR_{H}2 Domain, or CDR_{H}3 Domain, or any combination or sub-combination thereof sufficient to form a “gp41-Epitope-Binding Site” that permits the molecule to bind to an epitope of gp41. Preferably, however, such gp41-Binding Molecules will possess a CDR_{L}1 Domain, CDR_{L}2 Domain, CDR_{L}3 Domain, CDR_{H}1 Domain, CDR_{H}2 Domain and a CDR_{H}3 Domain of an “anti-gp41 Antibody” that immunospecifically binds an epitope of gp41. In some embodiments, such molecules may comprise only the gp41-VL Binding Domain of such anti-gp41 Antibody, or only the gp41-VH Binding Domain of such anti-gp41 Antibody, which domains may interact with other binding domains to mediate immunospecific binding to an epitope of gp41, or may be sufficient by themselves to mediate such binding. In some embodiments, such molecules may comprise both a gp41-VL Binding Domain of an anti-gp41 Antibody and a gp41-VH Binding Domain of an anti-gp41 Antibody, which may be derived from the same or different antibodies.

One embodiment of the present invention relates to “multispecific” gp41-Binding Molecules that are bispecific and are capable of binding to a “First Epitope” and a “Second Epitope,” such epitopes not being identical to one another. The denoting of an epitope as being the “First Epitope,” “Second Epitope,” etc. is intended merely for the purpose of providing antecedent basis to their description and does not signify a substantive distinction. Such multispecific molecules comprise “VL1”/“VH1” domains that are capable of binding to the First Epitope, and “VL2”/“VH2” domains that are capable of binding to the Second Epitope. The notation “VL1” and “VH1” denote respectively, the Variable Light Chain Domain and Variable Heavy Chain Domain that bind the First Epitope of such bispecific molecules. Similarly, the notation “VL2” and “VH2” denote respectively, the Light Chain Variable Domain and Heavy Chain Variable Domain that bind the Second Epitope of such bispecific molecules. It is irrelevant whether a particular epitope is designated as the First Epitope, the Second Epitope, etc.; such notation having relevance only with respect to the presence and orientation of domains of the polypeptide chains of the binding molecules of the present invention. In one embodiment, one of such epitopes is an epitope of human gp41 and the other is a different epitope of gp41, or is an epitope of a molecule that is not gp41. In particular embodiments, one of such epitopes is an epitope of human gp41 and the other is an epitope of a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, a multispecific molecule comprises more than two Epitope Binding Domains. In certain embodiments the multispecific molecules of the invention are trispecific and are capable of binding to a First Epitope, a Second Epitope, and a “Third Epitope.” Such trispecific molecules comprise, VL1/V111 domains that are capable of binding to the First Epitope, VL2/VH2 domains that are capable of binding to the Second Epitope, and “VL3” and “VH3” domains that are capable of binding to the Third Epitope. In other embodiments the multispecific molecules of the invention are tetraspecific and are capable of binding to a First Epitope, a Second Epitope, a Third Epitope, and a “Fourth Epitope.” Such tetraspecfic molecules and comprise, VL1/VH1 domains that are capable of binding to the First Epitope, VL2/VH2 domains that are capable of binding to the Second Epitope, VL3/VH3 domains that are capable of binding to the Third Epitope, and “VL4”/“VH4” domains that are capable of binding to the Fourth Epitope. Such multispecific molecules will bind at least one epitope of gp41 and at least one epitope of a molecule that is not gp41, and may further bind additional epitopes of gp41 and/or additional epitopes of a molecule that is not gp41. The instant invention particular encompasses bispecific diabodies, BiTE®s, antibodies, TandAb®s and trivalent molecules produced using any of the methods provided herein.

Multispecific gp41-Binding Molecules

In one embodiment, the gp41-Binding Molecules of the present invention will be multispecific gp41-Binding Molecules, capable of binding to two or more different epitopes, and will comprise:
(1) a first Epitope Binding Domain that immunospecifically binds an epitope of gp41 (i.e., a gp41-Binding Domain, preferably comprising an optimized gp41-binding VL Domain of the invention and/or an optimized gp41-binding VH Domain of the invention); and
(2) a second Epitope Binding Domain that immunospecifically binds a second epitope, where such second epitope is (i) a different epitope of gp41, or (ii) an epitope of a molecule that is not gp41; and
(3) optionally, one or more additional Epitope Binding Domains that immunospecifically bind a third additional epitope, a third additional epitope and a fourth additional epitope, etc., where such additional Epitope Binding Domains may be the same or different from the first epitope or the second epitope.

Such gp41-Binding Molecules preferably comprise a combination of Epitope Binding Domains that recognize a set of antigens unique to target cells or immune effector cells. In particular, the present invention relates to multispecific gp41-Binding Molecules that are capable of binding to an epitope of gp41 and to an epitope of a molecule present on the surface of an effector cell (i.e., effector cell molecule (“ECM”)), especially a molecule present on the surface of a T lymphocyte, a natural killer (NK) cell or other mononuclear cell. For example, such gp41-Binding Molecules of the present invention may be constructed to comprise a First Epitope Binding Domain that immunospecifically binds to an epitope of gp41 and a Second Epitope Binding Domain that immunospecifically binds to an epitope of an ECM, especially CD2, CD3, CD8, CD16, T-Cell Receptor (TCR), or NKG2D. In other embodiments of the invention, such binding molecules will additionally contain Epitope Binding Domains sufficient to permit such molecules to bind an additional epitope of an ECM and/or an additional epitope of a molecule present on the surface of an HIV-1 infected cell (e.g., a second epitope of gp41, or other epitope of an HIV-1 envelope protein). The present invention is also directed to pharmaceutical compositions that comprise such molecule(s).

In one embodiment, such gp41-Binding Molecules will be bispecific but monovalent so as to possess the ability to bind to only a single epitope of gp41 and only a single epitope of a molecule present on the surface of an effector cell (ECM). Alternatively, such molecules may be multispecific and multivalent, i.e., capable of binding one, two, three or four total epitopes, which may be apportioned in any manner to bind one, two or three epitope(s) of gp41 (which two or three gp41 epitopes may be the same or different) and three, two or one epitope(s) of one or more ECM; or one, or two epitope(s) of gp41 (which two gp41 epitopes may be the same or different) and two or one epitope(s) of one or more ECM, and optionally one, or two epitopes of one or more different HIV-1 molecule, particularly where such molecules(s) are expressed on the surface of an HIV-1 infected cell.

Thus, where such molecules are capable of immunospecifically binding to only a single ECM, they may be capable of immunospecifically binding to:
(1) only one gp41 epitope and to one, two or three epitope(s) of the ECM (which two epitopes may be the same or different, and which three epitopes may be the same, or may be different, or may be two epitopes that are the same and one epitope that is different), or
(2) only two gp41 epitopes (which two epitopes may be the same or different) and one or two epitope(s) of the ECM (which two epitopes may be the same or different); or
(3) three gp41 epitopes (which three epitopes may be the same, or may be different or may be two epitopes that are the same and one epitope that is different) and 1 epitope of the ECM, or optionally they may be capable of binding to one or two gp41 epitopes (which epitopes may be the same or different) and two or one epitopes of a different HIV-1 molecule (which two epitopes may be the same or different).

Similarly, where such molecules are capable of immunospecifically binding to two different ECMs (e.g., a First ECM and a Second ECM), they may be capable of immunospecifically binding to:
(1) only one gp41 epitope and to one or two epitope(s) of the First ECM (which two First ECM epitopes may be the same or different) and two or one epitope(s) of the Second ECM (which two Second ECM may be the same or different), or
(2) only two gp41 epitopes (which two epitopes may be the same or different) and one epitope of the First ECM and one epitope of the Second ECM, or optionally they may be capable of binding to one gp41 epitope and one epitope of a different HIV-1 molecule.

Non-limiting examples of such multispecific molecules capable of binding two epitopes are described below and include:
(1) DART-A, which binds one epitope of gp41, and one epitope of the ECM, CD3; and
(2) DART-B, which binds one epitope of gp41, and one epitope of the ECM, CD16.

Non-limiting examples of such multispecific molecules capable of binding three epitopes are described below and include:
(1) TRIDENT-A, which binds one epitope of gp41, and epitopes of two different ECMs (CD3 and CD8);
(2) TRIDENT-B, which binds one epitope of gp41, one epitope of an ECM (CD3), and one epitope of a different HIV-1 molecule (gp120);
(3) TRIDENT-C, which binds one epitope of gp41, one epitope of an ECM (CD16), and one epitope of a different HIV-1 molecule (gp120); and
(4) TRIDENT-D, which binds two epitopes of gp41, and one epitope of an ECM (CD3).

Table 1 further illustrates possible combination binding specificities of exemplary molecules of the invention.

TABLE 1
Number of Epitopes Recognized by Exemplary gp41-Binding Molecules of
the Invention Possessing Two, Three or Four Epitope Binding Domains
That Are Capable of Mediating the Redirected Killing of a Target Cell

| Total | | | | | No. of Epitopes |
|---|---|---|---|---|---|
| Number of | No. of | No. of | No. of | No. of | of Different |
| Binding | gp41 | Epitopes of | Epitopes of | Epitopes of | HIV-1 |
| Domains | Epitope(s) | 1^{st} ECM | 2^{nd} ECM | 3^{rd} ECM | molecule |
| 2 | 1 | 1 | 0 | 0 | 0 |
| 3 | 1 | 1 | 1 | 0 | 0 |
| 3 | 1 | 1 | 0 | 0 | 1 |
| 3 | 1 | 2 | 0 | 0 | 0 |
| 3 | 2 | 1 | 0 | 0 | 0 |
| 4 | 1 | 1 | 1 | 0 | 1 |
| 4 | 1 | 1 | 1 | 1 | 0 |
| 4 | 1 | 2 | 0 | 0 | 1 |
| 4 | 1 | 2 | 1 | 0 | 0 |
| 4 | 2 | 1 | 1 | 0 | 0 |
| 4 | 2 | 1 | 0 | 0 | 1 |

By forming more complex molecules, one may obtain gp41-Binding Molecules that are capable of binding one or more ECMs and optionally one or more different HIV-1 molecules that possess more than four epitope binding domains. Thus, no limitation is placed on the nature of epitopes or additional epitopes that may be bound by the molecules of the present invention other than that such additional binding capability does not prevent the molecule or Binding Domain thereof that is capable of binding to an epitope of gp41 from such binding and does not prevent the molecule or Binding Domain thereof that is capable of binding to an epitope of a ECM from such binding. Thus, the gp41 Binding Molecules of the present invention may possess alternative or additional Epitope Binding Domains. As an example, the invention contemplates a gp41 Binding Molecule that comprises a First Epitope Binding Domain capable of immunospecifically binding an epitope of gp41 and a Second Epitope Binding Domain that is capable of immunospecifically binding an epitope of a ECM and a Third Epitope Binding Domain capable of immunospecifically binding a different ECM or optionally a different HIV-1 molecule.

1. Multispecific gp41-Binding Diabodies Lacking Fc Domains

In one embodiment, the multispecific gp41-Binding Molecules of the present invention will be bispecific diabodies and will comprise domains capable of binding both a First Epitope and a Second Epitope, but will lack an Fc Domain, and thus will be unable to bind FcγR molecules via an Fc-FcγR interaction. Such molecules are, however, able to bind to gp41 via the SDRs or CDRs of their gp41 Binding Domains. The absence of Fc domains thus serves to prevent the molecules from binding to FcγRs, such as the inhibitory receptor CD32B.

The first polypeptide chain of such an embodiment of bispecific diabodies preferably comprises, in the N-terminal to C-terminal direction: an N-terminus, the VL Domain of a monoclonal antibody capable of binding either the First or Second Epitope (i.e., either VL_{gp41} or VL_{Second Epitope}), a first intervening spacer peptide (Linker 1), a VH Domain of a monoclonal antibody capable of binding the Second Epitope (if such first polypeptide chain contains VL_{gp41}) or a VH Domain of a monoclonal antibody capable of binding gp41 (if such first polypeptide chain contains VL_{Second Epitope}), a second intervening spacer peptide (Linker 2) optionally containing a cysteine residue, a Heterodimer-Promoting Domain and a C-terminus (FIGS. 1A-1B).

The second polypeptide chain of this embodiment of bispecific diabodies comprises, in the N-terminal to C-terminal direction: an N-terminus, the VL Domain of a monoclonal antibody capable of binding the First or Second Epitope (i.e., VL_{gp41} or VLSecond Epitope, and being the VL Domain not selected for inclusion in the first polypeptide chain of the diabody), an intervening spacer peptide (Linker 1), a VH Domain of a monoclonal antibody capable of binding either the First or Second Epitope (i.e., VH_{gp41} or VH_{Second Epitope}, and being the VH Domain not selected for inclusion in the first polypeptide chain of the diabody), a second intervening spacer peptide (Linker 2) optionally containing a cysteine residue, a Heterodimer-Promoting Domain and a C-terminus (FIGS. 1A-1B). The employed VL and VH Domains specific for a particular epitope are preferably obtained or derived from the same monoclonal antibody. However, such domains may be derived from different monoclonal antibodies provided that they associate to form a functional Binding Domain capable of immunospecifically binding such epitope. Such different antibodies are referred to herein as being “corresponding” antibodies.

The VL Domain of the first polypeptide chain of such diabodies interacts with the VH Domain of the second polypeptide chain of the diabody to form a first functional Epitope Binding Domain that is specific for one of the epitopes (e.g., the First Epitope or the Second Epitope). Likewise, the VL Domain of the second polypeptide chain interacts with the VH Domain of the first polypeptide chain in order to form a second functional Epitope Binding Domain that is specific for the other epitope (i.e., the Second Epitope or the First Epitope). Thus, the selection of the VL and VH Domains of the first and second polypeptide chains is “coordinated,” such that the two polypeptide chains of the diabody collectively comprise VL and VH Domains capable of binding both the First Epitope and the Second Epitope (i.e., they collectively comprise VL_{First Epitope}/VH_{First Epitope} and VL_{Second Epitope}/VH_{Second Epitope}), such as VL_{gp41}/VH_{gp41} and VL_{Second Epitope}/VH_{Second Epitope}.

Most preferably, the length of the intervening spacer peptide (i.e., “Linker 1”), which separates such VL and VH Domains, is selected to substantially or completely prevent the VL and VH Domains of the polypeptide chain from binding one another (for example consisting of 0, 1, 2, 3, 4, 5, 6, 7, 8 or 9 intervening linker amino acid residues). Thus, the VL and VH Domains of the first polypeptide chain are substantially or completely incapable of binding one another. Likewise, the VL and VH Domains of the second polypeptide chain are substantially or completely incapable of binding one another. A preferred intervening spacer peptide (Linker 1) has the sequence (SEQ ID NO:16): GGGSGGGG.

The length and composition of the second intervening spacer peptide (“Linker 2”) is selected based on the choice of one or more polypeptide domains that promote such dimerization (i.e., a “Heterodimer-Promoting Domain”). Typically, the second intervening spacer peptide (“Linker 2”) will be between 3 and 20 amino acid residues in length. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more than 3 cysteines. A preferred cysteine-containing spacer peptide (Linker 2) has the sequence GGCGGG (SEQ ID NO:17). Alternatively, Linker 2 does not comprise a cysteine (e.g., GGG, GGGS (SEQ ID NO:18), LGGGSG (SEQ ID NO:19), GGGSGGGSGGG (SEQ ID NO:20), ASTKG (SEQ ID NO:21), LEPKSS (SEQ ID NO:22), APSSS (SEQ ID NO:23), etc.) and a cysteine-containing Heterodimer-Promoting Domain, as described below is used. Optionally, both a cysteine-containing Linker 2 and a cysteine-containing Heterodimer-Promoting Domain are used.

The Heterodimer-Promoting Domains may be GVEPKSC (SEQ ID NO:24) or VEPKSC (SEQ ID NO:25) or AEPKSC (SEQ ID NO:26) on one polypeptide chain and GFNRGEC (SEQ ID NO:27) or FNRGEC (SEQ ID NO:28) on the other polypeptide chain (US2007/0004909).

In a preferred embodiment, the Heterodimer-Promoting Domains will comprise tandemly repeated coil domains of opposing charge for example, an “E-coil” Heterodimer-Promoting Domain (SEQ ID NO:29: EVAALEK-EVAALEK-EVAALEK-EVAALEK), whose glutamate residues will form a negative charge at pH 7, or a “K-coil” Heterodimer-Promoting Domain (SEQ ID NO:30: KVAALKE-KVAALKE-KVAALKE-KVAALKE), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence EVAA [symbol] EK-EVAALEK-EVAALEK-EVAALEK (SEQ ID NO:31), and a modified K-coil having the amino acid sequence KVAA [symbol] KE-KVAALKE-KVAALKE-KVAALKE (SEQ ID NO:32).

As disclosed in WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of a polypeptide chain of the diabody. Albumin is the most abundant protein in plasma and has a half-life of 19 days in humans. Albumin possesses several small molecule binding domains that permit it to non-covalently bind other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120). Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of Protein G of Streptococcus strain G148 (SEQ ID NO:33): LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:33 have the ability to attenuate or eliminate MHC class II binding. Based on combinational mutation results, the following combinations of substitutions are considered to be preferred substitutions for forming such a deimmunized ABD: 66D/70S+71A; 66S/70S+71A; 66S/70S+79A; 64A/65A/71A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 34)
LAEAKVLANR ELDKYGVSDY YKNLID₆₆NAKS₇₀ A₇₁EGVKALIDE
ILAALP,

or the amino acid sequence:

(SEQ ID NO: 35)
LAEAKVLANR ELDKYGVSDY YKNA₆₄A₆₅NNAKT VEGVKALIA₇₉E
ILAALP,

or the amino acid sequence:

(SEQ ID NO: 36)
LAEAKVLANR ELDKYGVSDY YKNLIS₆₆NAKS₇₀ VEGVKALIA₇₉E
ILAALP,

are particularly preferred as such deimmunized ABD exhibit substantially wild-type binding while providing attenuated MHC class II binding. Thus, the first polypeptide chain of such a diabody having an ABD contains a third linker (Linker 3) preferably positioned C-terminally to the E-coil (or K-coil) Domain of such polypeptide chain so as to intervene between the E-coil (or K-coil) Domain and the ABD (which is preferably a deimmunized ABD). A preferred sequence for such Linker 3 is SEQ ID NO:18: GGGS.

2. Multispecific gp41-Binding Diabodies Comprising Fc Domains

One embodiment of the present invention relates to multispecific diabodies (e.g., bispecific, trispecific, tetraspecific, etc.) that comprise an Fc Domain and that are capable of simultaneously binding an epitope of gp41 and a second epitope (e.g., an epitope of a different HIV-1 envelope protein, or an epitope expressed on the surface of an effector cell). The Fc Domain of such molecules may be of any isotype (e.g., IgG1, IgG2, IgG3, or IgG4). The molecules may further comprise a CH1 Domain and/or a Hinge Domain. When present, the CH1 Domain and/or Hinge Domain may be of any isotype (e.g., IgG1, IgG2, IgG3, or IgG4), and is preferably of the same isotype as the desired Fc Domain.

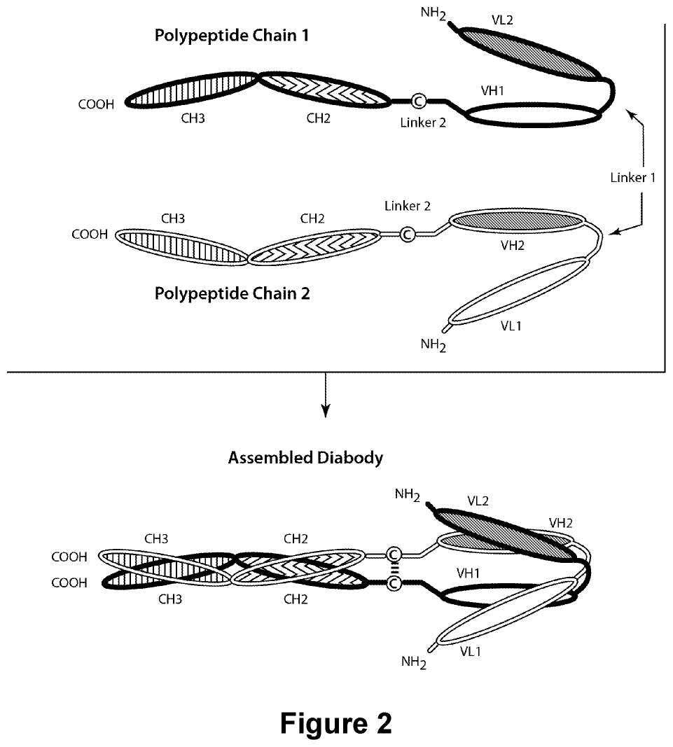

The addition of an IgG CH2-CH3 Domain to one or both of the diabody polypeptide chains, such that the complexing of the diabody chains results in the formation of an Fc Domain, increases the biological half-life and/or alters the valency of the diabody. Such diabodies comprise, two or more polypeptide chains whose sequences permit the polypeptide chains to covalently bind each other to form a covalently associated diabody that is capable of simultaneously binding gp41 and the Second Epitope. Incorporating an IgG CH2-CH3 Domains onto both of the diabody polypeptides will permit a two-chain bispecific Fc Domain-containing diabody to form (FIG. 2).

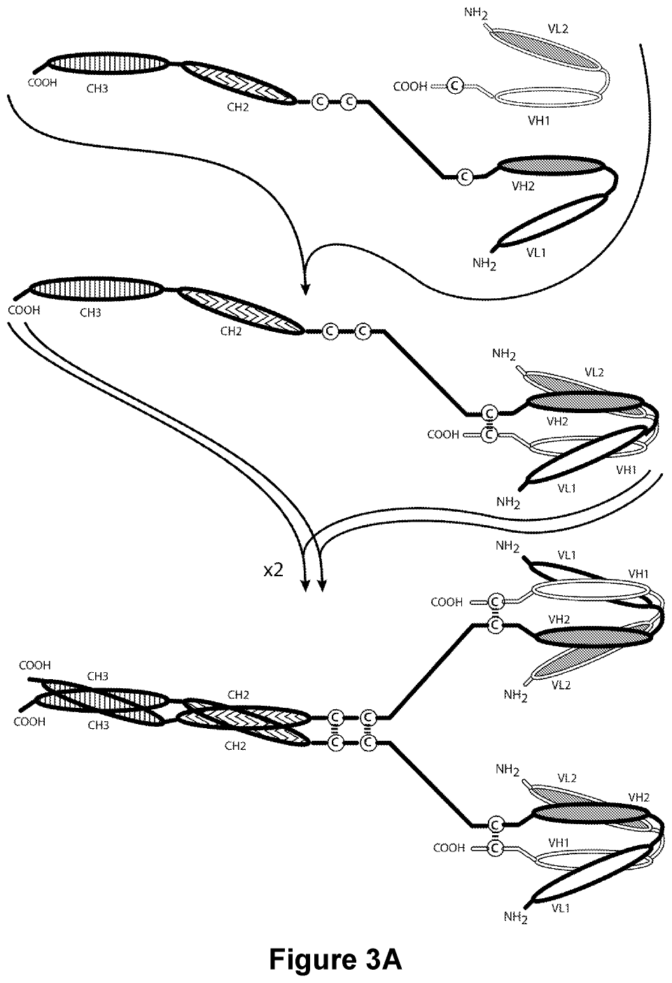

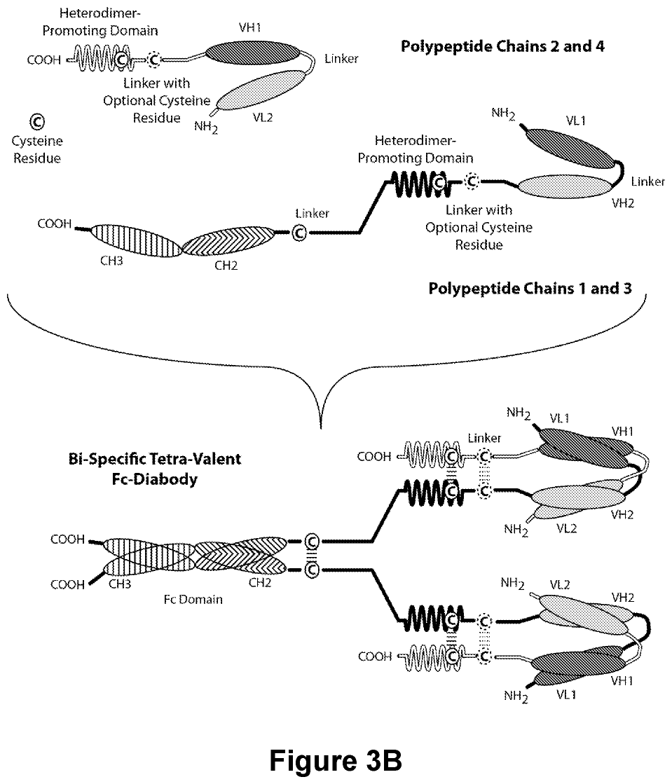

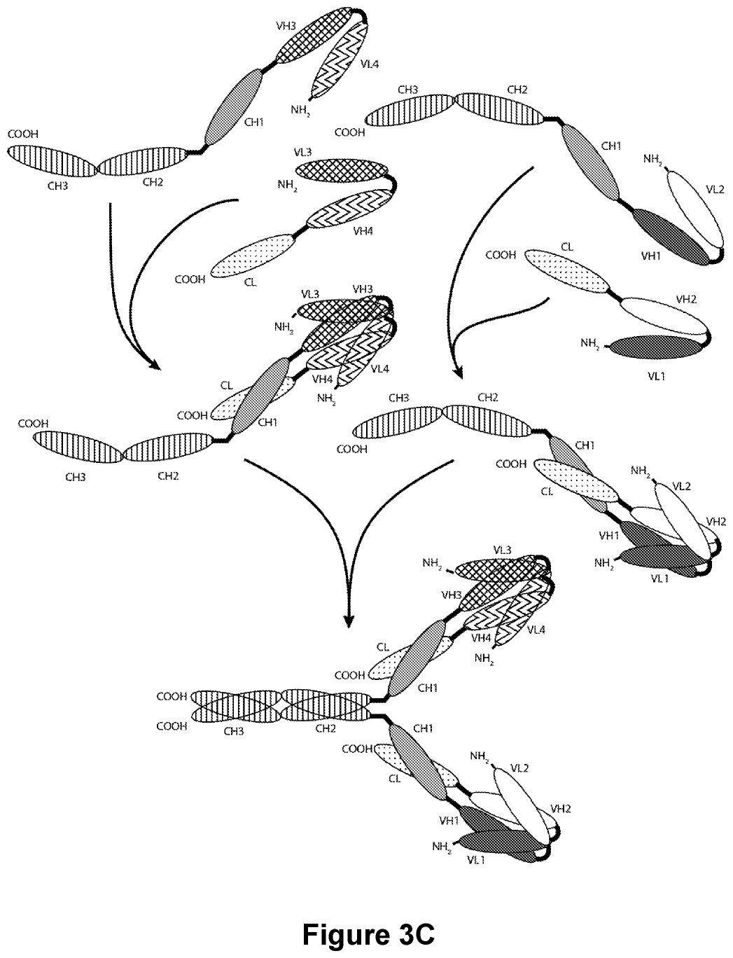

Alternatively, incorporating IgG CH2-CH3 Domains onto only one of the diabody polypeptides will permit a more complex four-chain bispecific Fc Domain-containing diabody to form (FIGS. 3A-3C). FIG. 3C shows a representative four-chain diabody possessing the Constant Light (CL) Domain and the Constant Heavy CH1 Domain, however portions of such domains as well as other polypeptides may alternatively be employed (see, e.g., FIGS. 3A and 3B, United States Patent Publication Nos. 2013-0295121; 2010-0174053 and 2009-0060910; European Patent Publication No. EP 2714079; EP 2601216; EP 2376109; EP 2158221 and PCT Publication Nos. WO 2012/162068; WO 2012/018687; WO 2010/080538). Thus, for example, in lieu of the CH1 Domain, one may employ a peptide having the amino acid sequence GVEPKSC (SEQ ID NO:24), VEPKSC (SEQ ID NO:25), or AEPKSC (SEQ ID NO:26), derived from the Hinge Domain of a human IgG, and in lieu of the CL Domain, one may employ the C-terminal 6 amino acids of the human kappa Light Chain, GFNRGEC (SEQ ID NO:27) or FNRGEC (SEQ ID NO:28). A representative peptide containing four-chain diabody is shown in FIG. 3A. Alternatively, or in addition, one may employ a peptide comprising tandem coil domains of opposing charge such as the “E-coil” helical domains (SEQ ID NO:29: EVAALEK-EVAALEK-EVAALEK-EVAALEK or SEQ ID NO:31: EVAAL [symbol] EK-EVAALEK-EVAALEK-EVAALEK); and the “K-coil” domains (SEQ ID NO:30: KVAALKE-KVAALKE-KVAALKE-KVAALKE or SEQ ID NO:32: KVAA [symbol] KE-KVAALKE-KVAALKE-KVAALKE). A representative coil domain containing four-chain diabody is shown in FIG. 3B.

Fc Domain-containing diabody molecules of the present invention may include additional intervening spacer peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Domain (preferably a cysteine-containing portion of an IgG Hinge Domain possessing 1, 2, 3 or more cysteine residues). Linkers that may be employed in the bispecific Fc Domain-containing diabody molecules of the present invention include: GGGS (SEQ ID NO:18), LGGGSG (SEQ ID NO:19), GGGSGGGSGGG (SEQ ID NO:20), AS TKG (SEQ ID NO:21), LEPKSS (SEQ ID NO:22), APSSS (SEQ ID NO:23), APSSSPME (SEQ ID NO:37), VEPKSADKTHTCPPCP (SEQ ID NO:38), LEPKSADKTHTCPPCP (SEQ ID NO:39), DKTHTCPPCP (SEQ ID NO:40), the scFv linker: GGGGSGGGGSGGGGS (SEQ ID NO:41), the “long” linker: GGGGSGGGSGGG (SEQ ID NO:42), GGC, and GGG. The linker LEPKSS (SEQ ID NO:22) may be used in lieu of GGG or GGC for ease of cloning. Additionally, the peptides GGG or LEPKSS (SEQ ID NO:22) may be immediately followed by DKTHTCPPCP (SEQ ID NO:40) to form the alternate linkers: GGGDKTHTCPPCP (SEQ ID NO:43); and LEPKSSDKTHTCPPCP (SEQ ID NO:44). Bispecific Fc Domain-containing molecules of the present invention may incorporate an IgG Hinge Domain in addition to or in place of a linker. Exemplary Hinge Domains include: EPKSCDKTHTCPPCP (SEQ ID NO:7) from IgG1, ERKCCVECPPCP (SEQ ID NO:8) from IgG2, ELKTPLGDTTHTCPRCPEPKSCDTPPPCPRCPEPKSCDTPP PCPRCPEPKSCDTPPPCPRCP (SEQ ID NO:9) from IgG3, ESKYGPPCPSCP (SEQ ID NO:10) from IgG4, and ESKYGPPCPPCP (SEQ ID NO:11), an IgG4 Hinge variant comprising a stabilizing S228P substitution (shown underlined) (as numbered by the EU index as set forth in Kabat) to reduce strand exchange.

As provided in FIG. 3A-3C, Fc Domain-containing diabodies of the invention may comprise four chains. The first and third polypeptide chains of such a diabody contain three domains: (i) a VL1-containing Domain, (ii) a VH2-containing Domain, (iii) a Heterodimer-Promoting Domain, and (iv) a Domain containing a CH2-CH3 sequence. The second and fourth polypeptide chains contain: (i) a VL2-containing Domain, (ii) a VH1-containing Domain, and (iii) a Heterodimer-Promoting Domain, where the Heterodimer-Promoting Domains promote the dimerization of the first/third polypeptide chains with the second/fourth polypeptide chains. The VL and/or VH Domains of the third and fourth polypeptide chains, and VL and/or VH Domains of the first and second polypeptide chains may be the same or different so as to permit tetravalent binding that is either mono-specific, bispecific or tetraspecific. The notation “VL3” and “VH3” denote respectively, the Light Chain Variable Domain and Variable Heavy Chain Domain that bind a “Third Epitope” of such diabody. Similarly, the notation “VL4” and “VH4” denote respectively, the Light Chain Variable Domain and Variable Heavy Chain Domain that bind a “Fourth Epitope” of such diabody. The general structure of the polypeptide chains of a representative four-chain bispecific Fc Domain-containing diabodies of invention is provided in Table 2:

TABLE 2

| | | |
|---|---|---|
| Bispecific | 2^{nd} Chain | NH₂-VL2-VH1-HPD-COOH |
| | 1^{st} Chain | NH₂-VL1-VH2-HPD-CH2—CH3—COOH |
| | 1^{st} Chain | NH₂-VL1-VH2-HPD-CH2—CH3—COOH |
| | 2^{nd} Chain | NH₂-VL2-VH1-HPD-COOH |
| Tetraspecific | 2^{nd} Chain | NH₂-VL2-VH1-HPD-COOH |
| | 1^{st} Chain | NH₂-VL1-VH2-HPD-CH2—CH3—COOH |
| | 3^{rd} Chain | NH₂-VL3-VH4-HPD-CH2—CH3—COOH |
| | 4^{th} Chain | NH₂-VL4-VH3-HPD-COOH |

HPD = Heterodimer-Promoting Domain

In a specific embodiment, diabodies of the present invention are bispecific, tetravalent (i.e., possess four Epitope Binding Domains), Fc-containing diabodies that are composed of four total polypeptide chains (FIGS. 3A-3C). The bispecific, tetravalent, Fc-containing diabodies of the invention comprise two First Epitope Binding Domains and two Second Epitope Binding Domains.

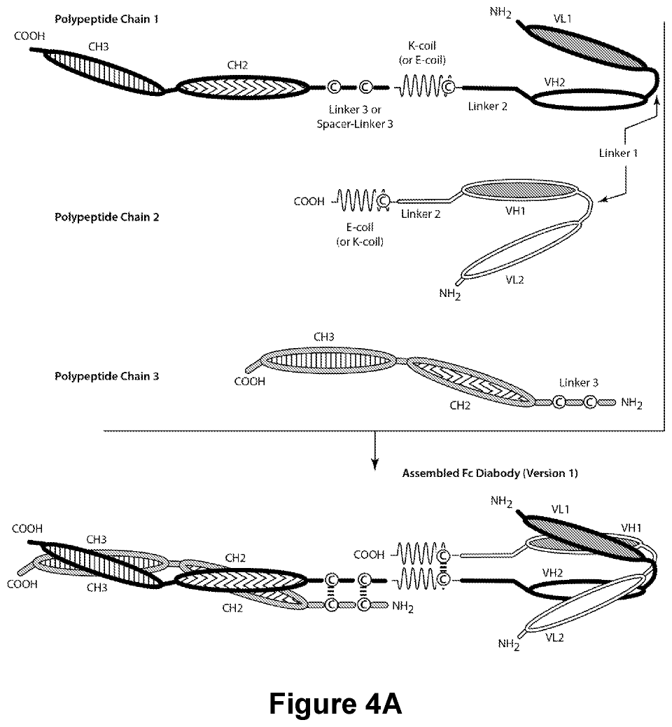

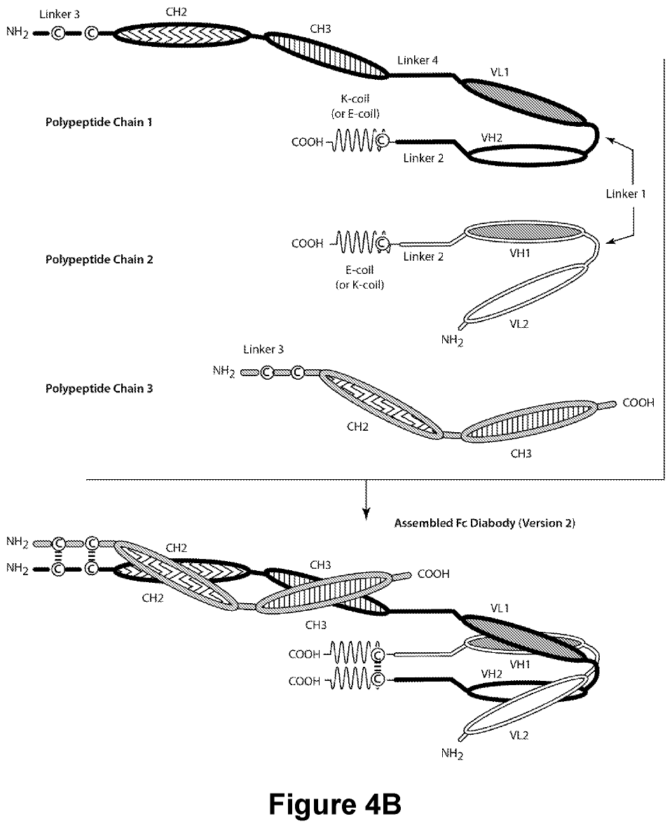

In a further embodiment, the Fc Domain-containing diabodies of the present invention may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 Epitope Binding Domain that is capable of binding either the First or Second Epitope, as well as a VL2/VH2 Epitope Binding Domain that is capable of binding the other of such epitopes. The first and second polypeptides are bonded to one another through a disulfide bond involving cysteine residues in their respective Third Domains. Notably, the first and third polypeptide chains complex with one another to form an Fc Domain that is stabilized via a disulfide bond. Such bispecific diabodies have enhanced potency. FIGS. 4A and 4B illustrate the structures of such diabodies. Such Fc Domain-containing diabodies may have either of two orientations (Table 3):

TABLE 3

| | | |
|---|---|---|
| First | 3^{rd} Chain | NH₂-CH2—CH3—COOH |
| Orientation | 1^{st} Chain | NH₂-VL1-VH2-HPD-CH2—CH3—COOH |
| | 2^{nd} Chain | NH₂-VL2-VH1-HPD-COOH |
| Second | 3^{rd} Chain | NH₂-CH2—CH3—COOH |
| Orientation | 1^{st} Chain | NH₂-CH2—CH3-VL1-VH2-HPD-COOH |
| | 2^{nd} Chain | NH₂-VL2-VH1-HPD-COOH |

HPD = Heterodimer-Promoting Domain

In a specific embodiment, diabodies of the present invention are bispecific, bivalent (i.e., possess two Epitope Binding Domains), Fc-containing diabodies that are composed of three total polypeptide chains (FIGS. 4A-4B). The bispecific, bivalent Fc-containing diabodies of the invention comprise one Epitope Binding Domain immunospecific for either the First or Second Epitope, as well as a VL2/VH2 Epitope Binding Domain that is capable of binding the other of such epitopes.

In a further embodiment, the Fc Domain-containing diabodies may comprise a total of five polypeptide chains. In a particular embodiment, two of the five polypeptide chains have the same amino acid sequence. The first polypeptide chain of such a diabody contains: (i) a VH1-containing Domain, (ii) a CH1-containing Domain, and (iii) a Domain containing a CH2-CH3 sequence. The first polypeptide chain may be the Heavy Chain of an antibody that contains a VH1 and a Heavy Chain constant region. The second and fifth polypeptide chains of such a diabody contain: (i) a VL1-containing Domain, and (ii) a CL-containing Domain. The second and/or fifth polypeptide chains of such a diabody may be Light Chains of an antibody that contains a VL1 complementary to the VH1 of the first/third polypeptide chain. The first, second and/or fifth polypeptide chains may be isolated from a naturally occurring antibody. Alternatively, they may be constructed recombinantly. The third polypeptide chain of such a diabody contains: (i) a VH1-containing Domain, (ii) a CH1-containing Domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain.

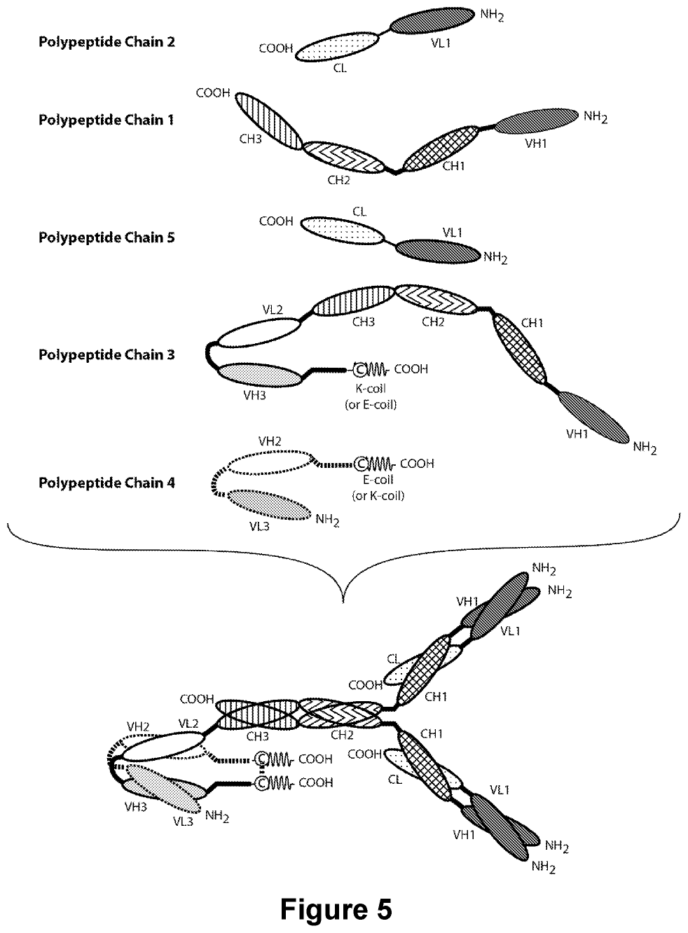

Thus, the first and second, and the third and fifth, polypeptide chains of such diabodies associate together to form two VL1/VH1 Epitope Binding Domains capable of binding a First Epitope. The third and fourth polypeptide chains of such diabodies associate together to form a VL2/VH2 Epitope Binding Domain that is capable of binding a Second Epitope, as well as a VL3/VH3 Epitope Binding Domain that is capable of binding a Third Epitope. The first and third polypeptides are bonded to one another through a disulfide bond involving cysteine residues in their respective constant regions. Notably, the first and third polypeptide chains complex with one another to form an Fc Domain. Such multispecific diabodies have enhanced potency. FIG. 5 illustrates the structure of such diabodies. It will be understood that the VL1/VH1, VL2/VH2, and VL3/VH3 Domains may be the same or different so as to permit binding that is mono-specific, bispecific or trispecific.

The VL and VH Domains of the polypeptide chains are selected so as to form VL/VH Epitope Binding Domains specific for a desired epitope. The VL/VH Epitope Binding Domains formed by the association of the polypeptide chains may be the same or different so as to permit tetravalent binding that is mono-specific, bispecific, trispecific or tetraspecific. In particular, the VL and VH Domains maybe selected such that a multivalent diabody may comprise two Binding Domains for a First Epitope and two Binding Domains for a Second Epitope, or three Binding Domains for a First Epitope and one Binding Domain for a Second Epitope, or two Binding Domains for a First Epitope, one Binding Domain for a Second Epitope and one Binding Domain for a Third Epitope (as depicted in FIG. 5). The general structure of the polypeptide chains of representative five-chain Fc Domain-containing diabodies of invention is provided in Table 4:

TABLE 4

| | | |
|---|---|---|
| Bispecific | 2^{nd} Chain | NH₂-VL1-CL-COOH |
| (2 × 2) | 1^{st} Chain | NH₂-VH1-CH1—CH2—CH3—COOH |
| | 3^{rd} Chain | NH₂-VH1-CH1—CH2—CH3-VL2-VH2-HPD-COOH |
| | 5^{nd} Chain | NH₂-VL1-CL-COOH |
| | 4^{th} Chain | NH₂-VL2-VH2-HPD-COOH |
| Bispecific | 2^{nd} Chain | NH₂-VL1-CL-COOH |
| (3 × 1) | 1^{st} Chain | NH₂-VH1-CH1—CH2—CH3—COOH |
| | 3^{rd} Chain | NH₂-VH1-CH1—CH2—CH3-VL1-VH2-HPD-COOH |
| | 5^{nd} Chain | NH₂-VL1-CL-COOH |
| | 4^{th} Chain | NH₂-VL2-VH1-HPD-COOH |
| Trispecific | 2^{nd} Chain | NH₂-VL1-CL-COOH |
| (2 × 1 × 1) | 1^{st} Chain | NH₂-VH1-CH1—CH2—CH3—COOH |
| | 3^{rd} Chain | NH₂-VH1-CH1—CH2—CH3-VL2-VH3-HPD-COOH |
| | 5^{nd} Chain | NH₂-VL1-CL-COOH |
| | 4^{th} Chain | NH₂-VL3-VH2-HPD-COOH |

HPD = Heterodimer-Promoting Domain

In a specific embodiment, diabodies of the present invention are bispecific, tetravalent (i.e., possess four Epitope Binding Domains), Fc-containing diabodies that are composed of five total polypeptide chains having two Epitope Binding Domains immunospecific for the First Epitope, and two Epitope Binding Domains specific for the Second Epitope. In another embodiment, the bispecific, tetravalent, Fc-containing diabodies of the invention comprise three Epitope Binding Domains immunospecific for the First Epitope and one Epitope Binding Domain specific for the Second Epitope. As provided above, the VL and VH Domains may be selected to permit trispecific binding. Accordingly, the invention also encompasses trispecific, tetravalent, Fc-containing diabodies. The trispecific, tetravalent, Fc-containing diabodies of the invention comprise two Epitope Binding Domains immunospecific for the First Epitope, one Epitope Binding Domain immunospecific for the Second Epitope, and one Epitope Binding Domain immunospecific for the Third Epitope.

In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell surface receptors on hematopoietic cells. As discussed above, the diversity of cellular responses triggered by antibodies and immune complexes results from the structural heterogeneity of the three Fc receptors: FcγRT (CD64), FcγRII (CD32), and FcγRIII (CD16). FcγRT (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:12), IgG2 (SEQ ID NO:13), IgG3 (SEQ ID NO:14), and IgG4 (SEQ ID NO:15) are presented above.

Modification of the Fc Domain may lead to an altered phenotype, for example altered serum half-life, altered stability, altered susceptibility to cellular enzymes or altered effector function. It may therefore be desirable to modify an Fc Domain-containing binding molecule of the present invention with respect to effector function, for example, so as to enhance the effectiveness of such molecule in treating cancer. Reduction or elimination of Fc Domain-mediated effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Molecules of the invention possessing such conferred or altered effector function activity are useful for the treatment and/or prevention of a disease, disorder or infection in which an enhanced efficacy of effector function activity is desired.

Accordingly, in certain embodiments, the Fc Domain of the gp41-Binding Fc Domain-containing Molecules of the present invention may be an engineered variant Fc Domain. Although the Fc Domain of the bispecific Fc Domain-containing molecules of the present invention may possess the ability to bind one or more Fc receptors (e.g., FcγR(s)), more preferably such variant Fc Domain have altered binding FcγRIA (CD64), FcγRIIA (CD32A), FcγRIIB (CD32B), FcγRIIIA (CD16A) or FcγRIIIB (CD16B) (relative to the binding exhibited by a wild-type Fc Domain), e.g., will have enhanced binding an activating receptor and/or will have substantially reduced or no ability to bind inhibitory receptor(s). Thus, the Fc Domain of the Fc Domain-containing molecules of the present invention may include some or all of the CH2 Domain and/or some or all of the CH3 Domain of a complete Fc Domain, or may comprise a variant CH2 and/or a variant CH3 sequence (that may include, for example, one or more insertions and/or one or more deletions with respect to the CH2 or CH3 domains of a complete Fc Domain). Such Fc Domains may comprise non-Fc polypeptide portions, or may comprise portions of non-naturally complete Fc Domains, or may comprise non-naturally occurring orientations of CH2 and/or CH3 Domains (such as, for example, two CH2 Domains or two CH3 Domains, or in the N-terminal to C-terminal direction, a CH3 Domain linked to a CH2 Domain, etc.).

Fc Domain modifications identified as altering effector function are known in the art, including modifications that increase binding activating receptors (e.g., FcγRIIA (CD16A) and reduce binding inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Table 5 lists exemplary single, double, triple, quadruple and quintuple substitutions (numbering (according to the EU index) and substitutions are relative to the amino acid sequence of SEQ ID NO:12 as presented above) of exemplary modification that increase binding activating receptors and/or reduce binding inhibitory receptors.

TABLE 5
Variations of Preferred Activating Fc Domains†
Single-Site Variations

| | | | |
|---|---|---|---|
| F243L | R292G | D270E | R292P |
| Y300L | P396L | | |

Double-Site Variations

| | | | |
|---|---|---|---|
| F243L and R292P | F243L and Y300L | F243L and P396L | R292P and Y300L |
| D270E and P396L | R292P and V305I | P396L and Q419H | P247L and N421K |
| R292P and P396L | Y300L and P396L | R255L and P396L | R292P and P305I |
| K392T and P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L | F243L, R292P, V305I, Y300L |
| and P396L | and P396L |
| L235P, F243L, R292P, Y300L | |
| and P396L | |

†numbering is according to the EU index as in Kabat

Exemplary variants of human IgG1 Fc Domains with reduced binding CD32B and/or increased binding CD16A contain F243L, R292P, Y300L, V3051 or P396L substitutions, wherein the numbering is that of the EU index as in Kabat. These amino acid substitutions may be present in a human IgG1 Fc Domain in any combination. In one embodiment, the variant human IgG1 Fc Domain contains a F243L, R292P and Y300L substitution. In another embodiment, the variant human IgG1 Fc Domain contains a F243L, R292P, Y300L, V3051 and P396L substitution.

In certain embodiments, it is preferred for the Fc Domains of the Fc Domain-containing binding molecules of the present invention to exhibit decreased (or substantially no) binding FcγRIA (CD64), FcγRIIA (CD32A), FcγRIIB (CD32B), FcγRIIIA (CD16A) or FcγRIIIB (CD16B) (relative to the binding exhibited by the wild-type IgG1 Fc Domain (SEQ ID NO:12). In a specific embodiment, the Fc Domain-containing binding molecules of the present invention comprise an IgG Fc Domain that exhibits reduced antibody-dependent cell-mediated cytotoxicity (ADCC) effector function. In a preferred embodiment, the CH2-CH3 Domains of such binding molecules include any 1, 2, 3, or 4 of the substitutions: L234A, L235A, D265A, N297Q, and N297G, wherein the numbering is that of the EU index as in Kabat. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc Domain that inherently exhibits decreased (or substantially no) binding FcγRIIIA (CD16A) and/or reduced effector function (relative to the binding and effector function exhibited by the wild-type IgG1 Fc Domain (SEQ ID NO:12)) is utilized. In a specific embodiment, the Fc Domain-containing binding molecules of the present invention comprise an IgG2 Fc Domain (SEQ ID NO:13), an IgG3 Fc Domain (SEQ ID NO:14) or an IgG4 Fc Domain (SEQ ID NO:15). When an IgG4 Fc Domain is utilized, the instant invention also encompasses the introduction of a stabilizing mutation, such as the Hinge Region S228P substitution described above (see, e.g., SEQ ID NO:11). Since the N297G, N297Q, L234A, L235A and D265A substitutions abolish effector function, in circumstances in which effector function is desired, these substitutions would preferably not be employed.

A preferred IgG1 sequence for the CH2 and CH3 Domains of the Fc Domain-containing molecules of the present invention having reduced or abolished effector function will comprise the substitutions L234A/L235A (SEQ ID NO:45):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is lysine (K) or is absent.

The serum half-life of proteins comprising Fc Domains may be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered.

In some embodiments, the Fc Domain-containing binding molecules of the present invention comprise a variant Fc Domain that comprises at least one amino acid modification relative to a wild-type Fc Domain, such that the molecule has an increased half-life (relative to such molecule if comprising a wild-type Fc Domain). In some embodiments, the Fc Domain-containing binding molecules of the present invention comprise a variant IgG Fc Domain that comprises a half-life extending amino acid substitution at one or more positions selected from the group consisting of 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436, wherein the numbering is that of the EU index as in Kabat. Numerous mutations capable of increasing the half-life of an Fc Domain-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and PCT Publication Nos. WO 98/23289; WO 2009/058492; and WO 2010/033279, which are herein incorporated by reference in their entireties.

In some embodiments, the Fc Domain-containing binding molecules of the present invention exhibiting enhanced half-life possess a variant Fc Domain comprising substitutions at two or more of Fc Domain residues 250, 252, 254, 256, 257, 288, 307, 308, 309, 311, 378, 428, 433, 434, 435 and 436. In particular, two or more substitutions selected from: T250Q, M252Y, S254T, T256E, K288D, T307Q, V308P, A378V, M428L, N434A, H435K, and Y436I, wherein the numbering is that of the EU index as in Kabat. In a specific embodiment, such molecules may possess a variant IgG Fc Domain comprising the substitution:
(A) M252Y, S254T and T256E;
(B) M252Y and S254T;
(C) M252Y and T256E;
(D) T250Q and M428L;
(E) T307Q and N434A;
(F) A378V and N434A;
(G) N434A and Y436I;
(H) V308P and N434A; or
(I) K288D and H435K.

In a preferred embodiment, a gp41-Binding Fc Domain-containing Molecule of the present invention possesses a variant IgG Fc Region comprising any 1, 2, or 3 of the substitutions: M252Y, S254T and T256E. The invention further encompasses gp41-Binding Molecules possessing variant Fc Regions comprising:

(A) one or more mutations which alter effector function and/or FcγR; and

(B) one or more mutations which extend serum half-life.

An IgG1 sequence for the CH2 and CH3 Domains of the Fc Domain-containing molecules of the present invention that provides an increased half-life (and that has a 10-fold increase in binding to both cynomolgus monkey and human FcRn) (Dall'Acqua, W. F. et al. (2006) “Properties of Human IgG1s Engineered for Enhanced Binding to the Neonatal Fc Receptor (FcRn),” J. Biol. Chem. 281(33):23514-23524) will comprise the substitutions M252Y/S254T/T256E (SEQ ID NO:46):

APELLGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is lysine (K) or is absent.

An alternative IgG1 sequence for the CH2 and CH3 Domains of the Fc Domain-containing molecules of the present invention combining the reduced or abolished effector function provided by the substitutions L234A/L235A and the increased serum half-life provided by the substitutions M252Y/S254T/T256E is provided by SEQ ID NO:47:

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein X is lysine (K) or is absent.

For certain antibodies, diabodies and trivalent binding molecules that are desired to have Fc-Domain-containing polypeptide chains of differing amino acid sequence (e.g., whose Fc Domain-containing polypeptide chains are desired to not be identical), it is desirable to reduce or prevent homodimerization from occurring between the CH2-CH3 Domains of identical chains (e.g., two first polypeptide chains or between the CH2-CH3 Domains of two third polypeptide chains). The CH2 and/or CH3 Domains of such polypeptide chains need not be identical in sequence, and advantageously are modified to foster heterodimer complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any pair of polypeptides comprising CH2-CH3 Domains that forms an Fc Domain to foster heterodimerization. Methods of protein engineering to favor heterodimerization over homodimerization are well-known in the art, in particular with respect to the engineering of immunoglobulin-like molecules, and are encompassed herein (see e.g., Ridgway et al. (1996) “‘Knobs-Into-Holes’ Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

A preferred knob is created by modifying an IgG Fc Domain to contain the modification T366W. A preferred hole is created by modifying an IgG Fc Domain to contain the modification T366S, L368A and Y407V. To aid in purifying a hole-bearing polypeptide chain homodimer from the final bispecific heterodimeric Fc Domain-containing molecule, the Protein A Binding Domain of the hole-bearing CH2 and CH3 Domains of a polypeptide chain is preferably mutated by amino acid substitution at position 435 (H435R). Thus, the hole-bearing polypeptide chain homodimer will not bind protein A, whereas the bispecific heterodimer will retain its ability to bind protein A via the Protein A Binding Domain. In an alternative embodiment, the hole-bearing polypeptide chain may incorporate amino acid substitutions at positions 434 and 435 (N434A/N435K).

A preferred IgG1 amino acid sequence for the CH2 and CH3 Domains of one Fc Domain-containing polypeptide chain of an Fc Domain-containing molecule of the present invention will have the “knob-bearing” sequence (SEQ ID NO:48):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is lysine (K) or is absent.

An alternative IgG1 amino acid sequence for the CH2 and CH3 Domains of one Fc Domain-containing polypeptide chain of an Fc Domain-containing molecule of the present invention having a M252Y/S254T/T256E substitution and a “knob-bearing” sequence is SEQ ID NO:49:

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is lysine (K) or is absent.

A preferred IgG1 amino acid sequence for the CH2 and CH3 Domains of the other Fc Domain-containing polypeptide chain of an Fc Domain-containing molecule of the present invention having two polypeptide chains (or the third polypeptide chain of an Fc Domain-containing molecule having three, four, or five polypeptide chains) will have the “hole-bearing” sequence (SEQ ID NO:50):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is lysine (K) or is absent.

An alternative IgG1 amino acid sequence for the CH2 and CH3 Domains of the other Fc Domain-containing polypeptide chain of an Fc Domain-containing molecule of the present invention having a M252Y/S254T/T256E substitution and a “hole-bearing” sequence is SEQ ID NO:51:

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSTWQQG NVFSCSVMHE
ALHNRYTQKS LSLSPGX

wherein X is lysine (K) or is absent.

An IgG4 amino acid sequence for the CH2 and CH3 Domains of the one Fc Domain-containing polypeptide chain of an Fc Domain-containing molecule of the present invention has enhanced serum half-life (relative to IgG1 CH2 and CH3 Domains) due to its possession of Y252/T254/E256 (SEQ ID NO:52):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGX

wherein X is lysine (K) or is absent.

A “knob-bearing” variant of such an IgG4 CH2-CH3 amino acid sequence has the amino acid sequence of SEQ ID NO:53:

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLWCLVK GFYPSDIAVE WESNGQPENN
YKTTPPVLDS DGSFFLYSRL TVDKSRWQEG NVFSCSVMHE
ALHNHYTQKS LSLSLGX

wherein X is lysine (K) or is absent.

A “hole-bearing” variant of such an IgG4 CH2-CH3 amino acid sequence has the amino acid sequence of SEQ ID NO:54:

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLSCAVK GFYPSDIAVE WESNGQPENN
YKTTPPVLDS DGSFFLVSRL TVDKSRWQEG NVFSCSVMHE
ALHNRYTQKS LSLSLGX

wherein X is lysine (K) or is absent.

As will be noted, the CH2-CH3 Domains of SEQ ID NO:51, SEQ ID NO:52, SEQ ID NO:53, and SEQ ID NO:54 include a substitution at position 234 with alanine and 235 with alanine, and thus form an Fc Domain exhibit decreased (or substantially no) binding FcγRIA (CD64), FcγRIIA (CD32A), FcγRIIB (CD32B), FcγRIIIA (CD16A) or FcγRIIIB (CD16B) (relative to the binding exhibited by the wild-type Fc Domain (SEQ ID NO:12). The invention also encompasses such CH2-CH3 Domains, which comprise the wild-type alanine residues, alternative and/or additional substitutions which modify effector function and/or FγR binding activity of the Fc Domain. The invention also encompasses such CH2-CH3 Domains, which further comprise one or more half-live extending amino acid substitutions. In particular, the invention encompasses such hole-bearing and such knob-bearing CH2-CH3 Domains which further comprise the M252Y/S254T/T256E.

It is preferred that the first polypeptide chain will have a “knob-bearing” CH2-CH3 sequence, such as that of SEQ ID NO:48 or SEQ ID NO:49. However, as will be recognized, a “hole-bearing” CH2-CH3 Domain (e.g., SEQ ID NO:50 or SEQ ID NO:51) could be employed in the first polypeptide chain, in which case, a “knob-bearing” CH2-CH3 Domain (e.g., SEQ ID NO:48 or SEQ ID NO:49) would be employed in the second polypeptide chain of an Fc Domain-containing molecule of the present invention having two polypeptide chains (or in the third polypeptide chain of an Fc Domain-containing molecule having three, four, or five polypeptide chains).

In other embodiments, the invention encompasses Fc Domain-containing binding molecules comprising CH2 and/or CH3 Domains that have been engineered to favor heterodimerization over homodimerization using mutations known in the art, such as those disclosed in PCT Publication No. WO 2007/110205; WO 2011/143545; WO 2012/058768; WO 2013/06867; WO 2014/081955; WO 2016/086189, all of which are incorporated herein by reference in their entirety.

3. Trivalent Binding Molecules Containing Fc Domains

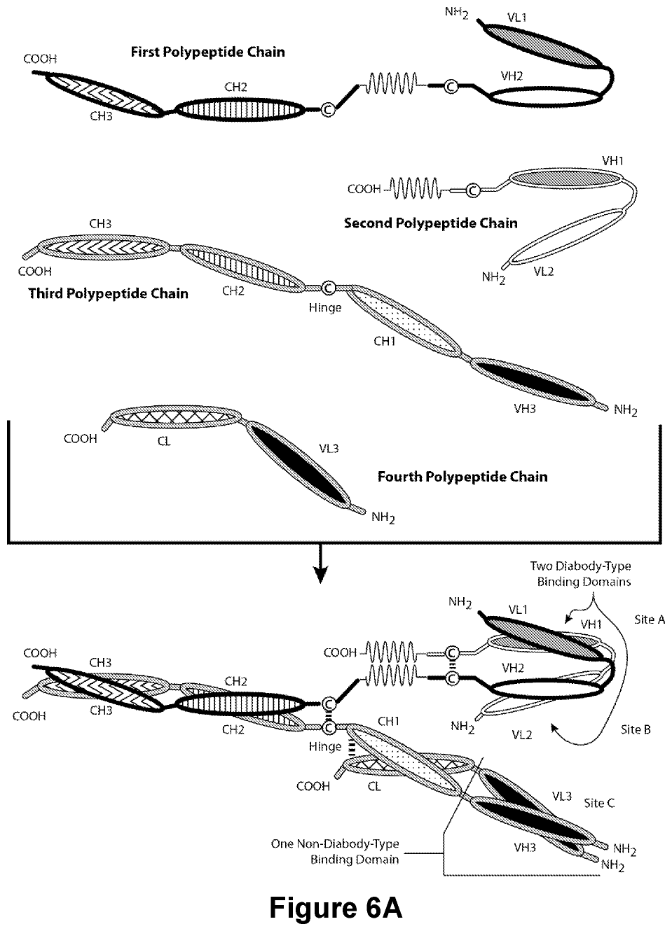

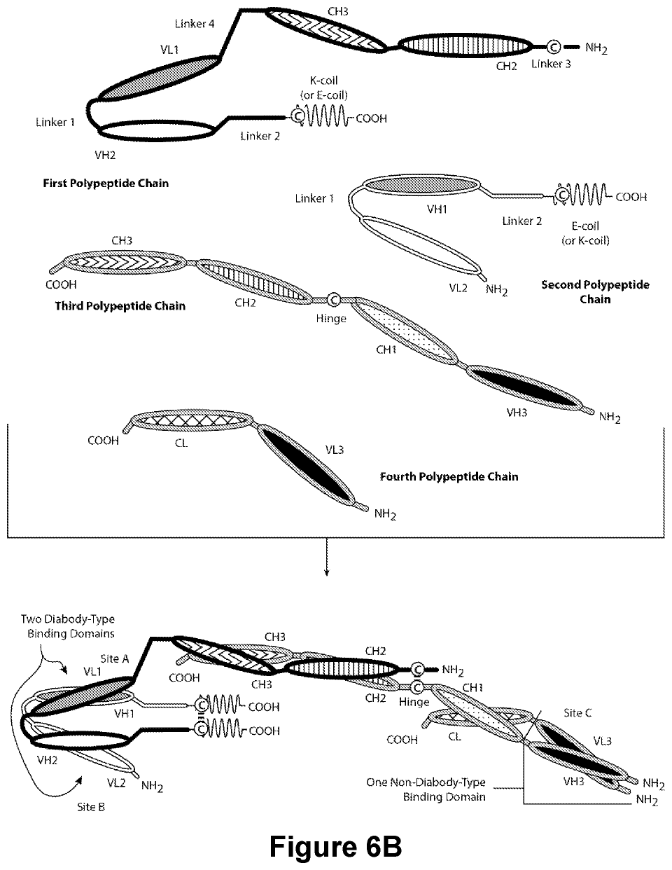

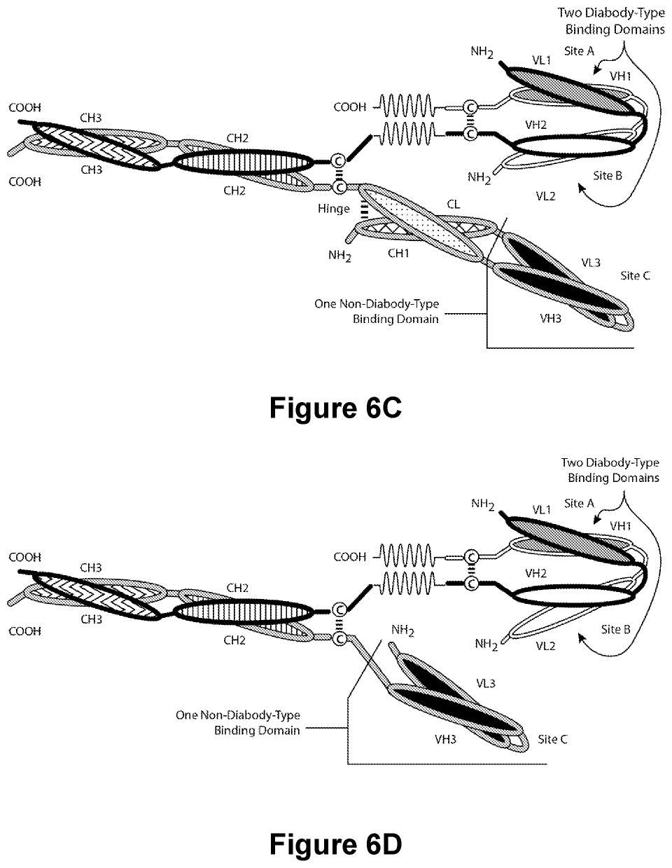

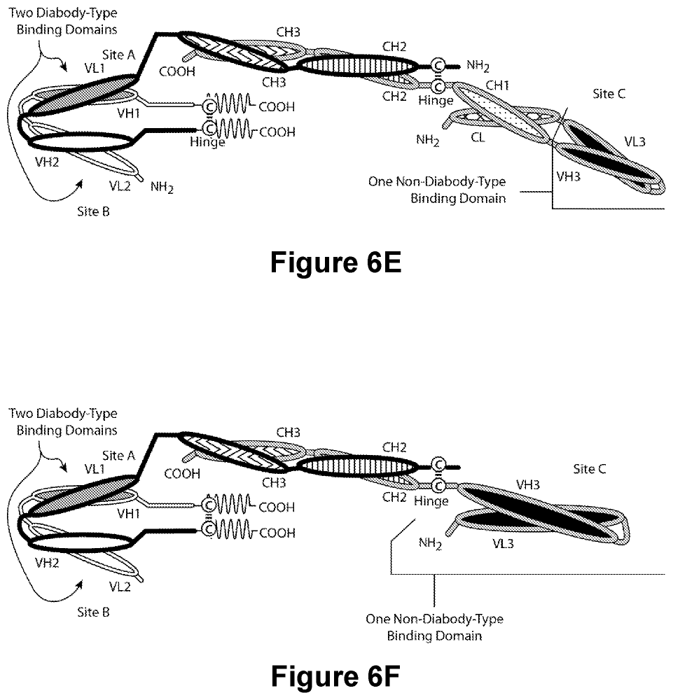

A further embodiment of the present invention relates to trivalent binding molecules that comprise an Fc Domain and are capable of simultaneously binding a First Epitope, a Second Epitope and a Third Epitope, wherein at least one of such epitopes is not identical to another. Such trivalent binding molecules comprise three Epitope Binding Domains, two of which are Diabody-Type Binding Domains, which provide Binding Domain A and Binding Domain B, and one of which is a Fab-Type Binding Domain, or an scFv-Type Binding Domain, which provides Binding Domain C (see, e.g., FIGS. 6A-6F, PCT Publication Nos. WO 2015/184207 and WO 2015/184203). Such trivalent binding molecules thus comprise “VL1”/“VH1” domains that are capable of binding the First Epitope and “VL2”/“VH2” domains that are capable of binding the Second Epitope and “VL3” and “VH3” domains that are capable of binding the Third Epitope of such trivalent binding molecule. A “Diabody-Type Binding Domain” is the type of Epitope Binding Domain present in a diabody, as described above, wherein a single polypeptide chain comprises both a VL Domain and a VH Domain, but such domains do not interact to form an Epitope Binding Site. Each of a “Fab-Type Binding Domain” and an “scFv-Type Binding Domain” is the type of Epitope Binding Domain present in an antibody, as described above, wherein a single polypeptide chain comprises (1) either a VL Domain or a VH Domain, and a second polypeptide chain comprises the corresponding VH Domain or VL Domain so as to form an Epitope Binding Site, or (2) both a VL Domain and a VH Domain, wherein such domains interact to form an Epitope Binding Site. Fab-Type Binding Domains also differ from Diabody-Type Binding Domains in that the two polypeptide chains that form a Fab-Type Binding Domain comprise only a single Epitope Binding Domain, whereas the two polypeptide chains that form a Diabody-Type Binding Domain comprise at least two Epitope Binding Domains. Similarly, scFv-Type Binding Domains also differ from Diabody-Type Binding Domains in that they comprise only a single Epitope Binding Domain. Thus, as used herein Fab-Type, and scFv-Type Binding Domains are distinct from Diabody-Type Binding Domains.

Typically, the trivalent gp41-Binding Molecules of the present invention will comprise four different polypeptide chains (see FIGS. 6A-6B), however, the molecules may comprise fewer or greater numbers of polypeptide chains, for example by fusing such polypeptide chains to one another (e.g., via a peptide bond) or by dividing such polypeptide chains to form additional polypeptide chains, or by associating fewer or additional polypeptide chains via disulfide bonds. FIGS. 6C-6F illustrate this aspect of the present invention by schematically depicting such molecules having three polypeptide chains. As provided in FIGS. 6A-6F, the trivalent binding molecules of the present invention may have alternative orientations in which the Diabody-Type Binding Domains are N-terminal (FIGS. 6A, 6C, and 6D) or C-terminal (FIGS. 6B, 6E and 6F) to an Fc Domain. CH2 and CH3 Domains useful for the generation of trivalent binding molecules are provided above and include knob-bearing and hole-bearing domains.

In certain embodiments, the first polypeptide chain of such trivalent gp41-Binding Molecules of the present invention contains: (i) a VL1-containing Domain, (ii) a VH2-containing Domain, (iii) a Heterodimer-Promoting Domain, and (iv) a Domain containing a CH2-CH3 sequence. The VL1 and VL2 Domains are located N-terminal or C-terminal to the CH2-CH3-containing domain as presented in Table 5 (also see, FIGS. 6A and 6B). The second polypeptide chain of such embodiments contains: (i) a VL2-containing Domain, (ii) a VH1-containing Domain, and (iii) a Heterodimer-Promoting Domain. The third polypeptide chain of such embodiments contains: (i) a VH3-containing Domain, (ii) a CH1-containing Domain and (iii) a Domain containing a CH2-CH3 sequence. The third polypeptide chain may be the Heavy Chain of an antibody that contains a VH3 and a Heavy Chain constant region, or a polypeptide that contains such domains. The fourth polypeptide of such embodiments contains: (i) a VL3-containing Domain and (ii) a CL-containing Domain. The fourth polypeptide chains may be a Light Chain of an antibody that contains a VL3 complementary to the VH3 of the third polypeptide chain, or a polypeptide that contains such domains. The third or fourth polypeptide chains may be isolated from naturally occurring antibodies. Alternatively, they may be constructed recombinantly, synthetically or by other means.

The Light Chain Variable Domain of the first and second polypeptide chains are separated from the Heavy Chain Variable Domains of such polypeptide chains by an intervening spacer peptide having a length that is too short to permit their VL1/VH2 (or their VL2/VH1) domains to associate together to form an Epitope Binding Domain capable of binding either the First or Second Epitope. A preferred intervening spacer peptide (Linker 1) for this purpose has the sequence (SEQ ID NO:16): GGGSGGGG. Other Domains of the trivalent binding molecules may be separated by one or more intervening spacer peptides (Linkers), optionally comprising a cysteine residue. In particular, as provided above, such Linkers will typically be incorporated between Variable Domains (i.e., VH or VL) and peptide Heterodimer-Promoting Domains (e.g., an E-coil or K-coil) and between such peptide Heterodimer-Promoting Domains (e.g., an E-coil or K-coil) and CH2-CH3 Domains. Exemplary linkers useful for the generation of trivalent binding molecules are provided above and are also provided in International Patent Publication Nos: WO 2015/184207; and WO 2015/184203. Thus, the first and second polypeptide chains of such trivalent binding molecules associate together to form a VL1/VH1 Binding Domain capable of binding a First Epitope, as well as a VL2/VH2 Binding Domain that is capable of binding a Second Epitope. The third and fourth polypeptide chains of such trivalent binding molecules associate together to form a VL3/VH3 Binding Domain that is capable of binding a Third Epitope.

As described above, the trivalent gp41-Binding Molecules of the present invention may comprise three polypeptides. Trivalent binding molecules comprising three polypeptide chains may be obtained by linking the domains of the fourth polypeptide N-terminal to the VH3-containing Domain of the third polypeptide (e.g., using an intervening spacer peptide (Linker 4)). Alternatively, a third polypeptide chain of a trivalent binding molecule of the invention containing the following domains is utilized: (i) a VL3-containing Domain, (ii) a VH3-containing Domain, and (iii) a Domain containing a CH2-CH3 sequence, wherein the VL3 and VH3 are spaced apart from one another by an intervening spacer peptide that is sufficiently long (at least 9 or more amino acid residues) so as to allow the association of these domains to form an Epitope Binding Domain. One preferred intervening spacer peptide for this purpose has the sequence: GGGGSGGGGSGGGGS (SEQ ID NO:41).

It will be understood that the VL1/VH1, VL2/VH2, and VL3/VH3 Domains of such trivalent binding molecules may be different so as to permit binding that is mono-specific, bispecific or trispecific. In particular, the VL and VH Domains may be selected such that a trivalent binding molecule comprises two Binding Domains for a First Epitope and one Binding Domains for a Second Epitope, or one Binding Domain for a First Epitope and two Binding Domains for a Second Epitope, or one Binding Domain for a First Epitope, one Binding Domain for a Second Epitope and one Binding Domain for a Third Epitope.

The general structure of the polypeptide chains of representative trivalent binding molecules of invention is provided in FIGS. 6A-6H and in Table 6:

TABLE 6

| | | |
|---|---|---|
| Four Chain | 2^{nd} Chain | NH₂-VL2-VH1-HPD-COOH |
| 1^{st} | 1^{st} Chain | NH₂-VL1-VH2-HPD-CH2—CH3—COOH |
| Orientation | 3^{rd} Chain | NH₂-VH3-CH1—CH2—CH3—COOH |
| | 2^{nd} Chain | NH₂-VL3-CL-COOH |
| Four Chain | 2^{nd} Chain | NH₂-VL2-VH1-HPD-COOH |
| 2nd | 1^{st} Chain | NH₂-CH2—CH3-VL1-VH2-HPD-COOH |
| Orientation | 3^{rd} Chain | NH₂-VH3-CH1—CH2—CH3—COOH |
| | 2^{nd} Chain | NH₂-VL3-CL-COOH |
| Three Chain | 2^{nd} Chain | NH₂-VL2-VH1-HPD-COOH |
| 1st | 1^{st} Chain | NH₂-VL1-VH2-HPD-CH2—CH3—COOH |
| Orientation | 3^{rd} Chain | NH₂-VL3-VH3-HPD-CH2—CH3—COOH |
| Three Chain | 2^{nd} Chain | NH₂-VL2-VH1-HPD-COOH |
| 2^{nd} | 1^{st} Chain | NH₂-CH2—CH3-VL1-VH2-HPD-COOH |
| Orientation | 3^{rd} Chain | NH₂-VL3-VH3-HPD-CH2—CH3—COOH |

HPD = Heterodimer-Promoting Domain

As provided above, such trivalent binding molecules may comprise three, four, five, or more polypeptide chains.

### IV. Exemplary gp41-Binding Molecules of the Present Invention

The present invention is directed to gp41-Binding Molecules (e.g., an antibody, a diabody, an scFv, an antibody, a TandAb®, a Trident™, etc.) capable of binding an epitope of the gp41 HIV-1 envelope (Env) protein by virtue of their possession of an optimized gp41 Binding Domain having the binding specificity of the 7B2 antibody.

A. The Envelope (Env) Glycoprotein of HIV

The envelope (Env) glycoprotein of HIV is expressed on the surface of productively infected cells. HIV type 1 (HIV-1) enters the host through the mucosa in all transmissions in a process known as transcytosis (Shen, R. et al. (2010) “GP41-Specific Antibody Blocks Cell-Free HIV Type 1 Transcytosis Through Human Rectal Mucosa And Model Colonic Epithelium,” J. Immunol. 184(7):3648-3655). HIV-1 transcytosis across gut and genital epithlelium has been reported to involve viral components, including the gp41 env protein (Bomsel, M. et al. (1998) “Intracellular Neutralization Of HIV Transcytosis Across Tight Epithelial Barriers By Anti-HIV Envelope Protein dIgA Or IgM,” Immunity 9:277-287; Alfsen, A. et al. (2002) “HIV-1 Gp41 Envelope Residues 650-685 Exposed On Native Virus Act As A Lectin To Bind Epithelial Cell Galactosyl Ceramide,” J. Biol. Chem. 277:25649-25659; Alfsen, A. et al. (2001) “Secretory IgA Specific For A Conserved Epitope On gp41 Envelope Glycoprotein Inhibits Epithelial Transcytosis Of HIV-1,” J. Immunol. 166:6257-6265; Tudor, D. et al. (2009) “HIV-1 gp41-Specific Monoclonal Mucosal IgAs Derived from Highly Exposed But IgG-Seronegative Individuals Block HIV-1 Epithelial Transcytosis And Neutralize CD4+ Cell Infection: An IgA Gene And Functional Analysis,” Mucosal Immunol. 2:412-426), gp120, and gp160, and host epithelial cell receptor and attachment molecules, including the glycosphingolipid galactosylceramide, the coreceptor CCRS, and the heparin sulfate proteoglycan attachment receptors, syndecan and agrin (Shen, R. et al. (2010) “GP41-Specific Antibody Blocks Cell-Free HIV Type 1 Transcytosis Through Human Rectal Mucosa and Model Colonic Epithelium,” J. Immunol. 184(7):3648-3655.

Thus, gp41 can be used as a target for cytotoxic immunoconjugates (ICs), in which cell-killing moieties, including toxins, drugs, or radionuclides, are chemically or genetically linked to gp41-Binding Molecules (see, e.g., Pincus, S. H. et al. (2017) “Design and In Vivo Characterization of Immunoconjugates Targeting HIV gp160,” J. Virol. 91(3). pii: e01360-16. doi: 10.1128/JVI.01360-16).

B. Antibody 7B2

Antibody 7B2 (Genbank accession numbers JX188438 and JX188439) is an anti-HIV env human IgG1 antibody that binds HIV gp41 at 598-604 in the immunodominant helix-loop-helix region of the molecule (Sadraeian, M. et al. (2017) “Selective Cytotoxicity Of A Novel Immunotoxin Based On Pulchellin A Chain For Cells Expressing HIV Envelope,” Sci. Rep. 7(1):7579 doi: 10.1038/s41598-017-08037-3). The antibody was isolated from an HIV-1 chronically infected subject using Epstein-Barr (EB) virus B cell transformation and heterohybridoma production (Pincus, S. H. et al. (2003) “In Vivo Efficacy Of Anti-Glycoprotein 41, But Not Anti-Glycoprotein 120, Immunotoxins In A Mouse Model Of HIV Infection,” J. Immunol. 170(4):2236-2241). Antibody 7B2 has been found to be capable of recognizing both virus particles and infected cells (Santra, S. et al. (2015) “Human Non-neutralizing HIV-1 Envelope Monoclonal Antibodies Limit the Number of Founder Viruses during SHIV Mucosal Infection in Rhesus Macaques,” PLoS Pathog. 11(8):e1005042. doi: 10.1371/journal.ppat.1005042; Tay, M. Z. et al. (2016) “Antibody-Mediated Internalization of Infectious HIV-1 Virions Differs among Antibody Isotypes and Subclasses,” PLoS Pathog. 12(8):e1005817. doi: 10.1371/journal.ppat.1005817).

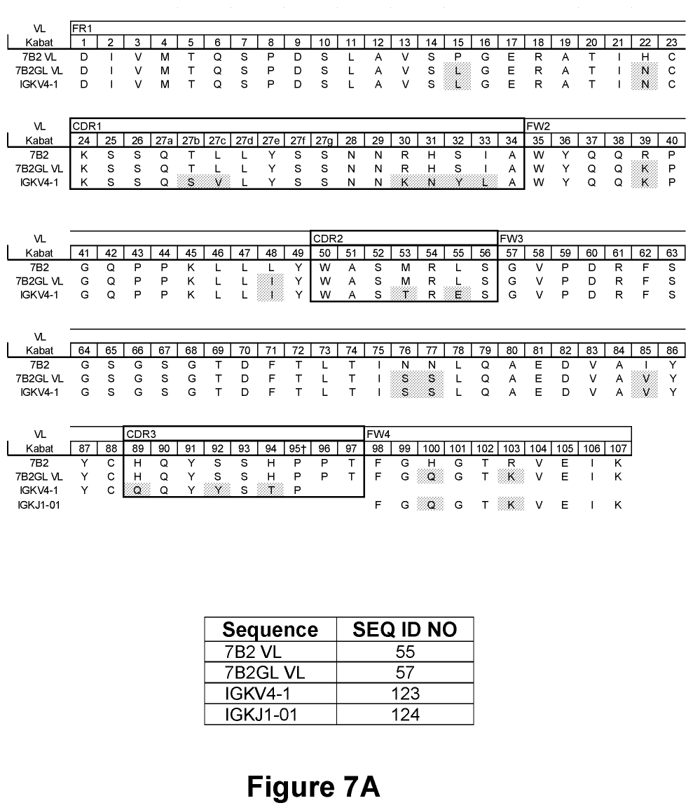

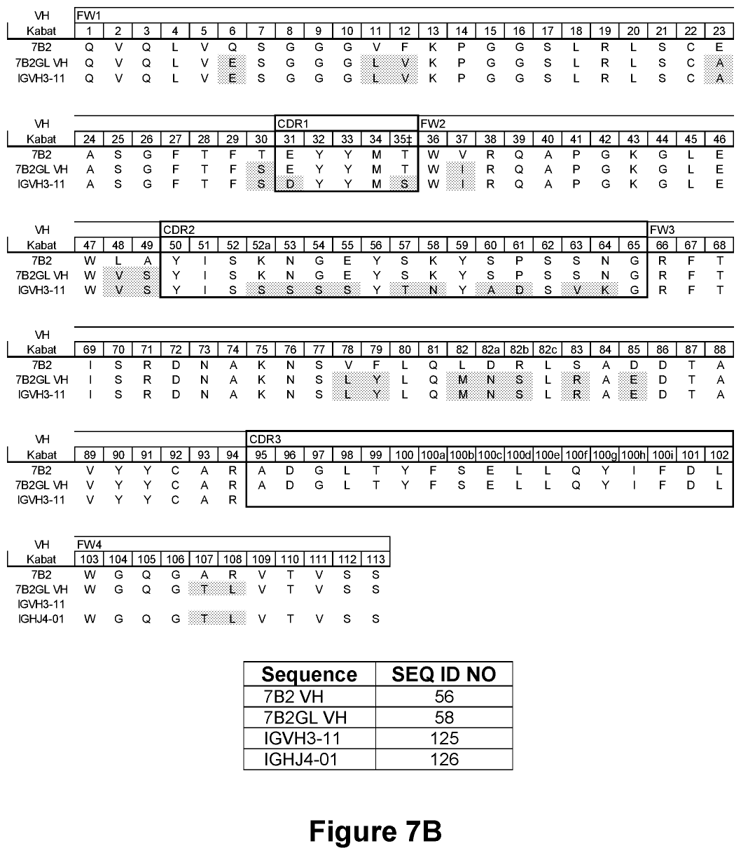

The amino acid sequence of the VL Domain of the 7B2 antibody (SEQ ID NO:55) is shown below and in FIG. 7A (CDR_{L} residues are shown underlined):

DIVMTQSPDS LAVSPGERAT IHCKSSQTLL YSSNNRHSIA
WYQQRPGQPP KLLLYWASMR LSGVPDRFSG SGSGTDFTLT
INNLQAEDVA IYYCHQYSSH PPTFGHGTRV EIK

The amino acid sequence of the VH Domain of the 7B2 antibody (SEQ ID NO:56) is shown below and in FIG. 7B (CDR_{H} residues are shown underlined):

QVQLVQSGGG VFKPGGSLRL SCEASGFTFT EYYMTWVRQA
PGKGLEWLAY ISKNGEYSKY SPSSNGRFTI SRDNAKNSVF
LQLDRLSADD TAVYYCARAD GLTYFSELLQ YIFDLWGQGA
RVTVSS

C. Optimized 7B2GL

The amino acid sequence of the VL Domain of the optimized 7B2GL antibody (SEQ ID NO:57) is shown below and in FIG. 7A (CDR_{L} residues are shown underlined):

DIVMTQSPDS LAVSLGERAT INCKSSQTLL YSSNNRHSIA
WYQQKPGQPP KLLIYWASMR LSGVPDRFSG SGSGTDFTLT
ISSLQAEDVA VYYCHQYSSH PPTFGQGTKV EIK

The amino acid sequence of the VH Domain of the optimized 7B2GL antibody

(SEQ ID NO:58) is shown below and in FIG. 7B (CDR_{H} residues are shown underlined):

QVQLVESGGG LVKPGGSLRL SCAASGFTFS EYYMTWIRQA
PGKGLEWVSY ISKNGEYSKY SPSSNGRFTI SRDNAKNSLY
LQMNSLRAED TAVYYCARAD GLTYFSELLQ YIFDLWGQGT
LVTVSS

### V. Exemplary Effector Cell Binding Capabilities

As provided herein, the present invention relates to multispecific gp41-Binding Molecules that are capable of binding to an epitope of gp41 and to an epitope of an effector cell surface molecule (“ECM”). As used herein, the term “effector cell” denotes a cell that directly or indirectly mediates the killing of target cells (e.g., foreign cells, infected cells or cancer cells). Examples of effector cells include helper T Cells, cytotoxic T Cells, Natural Killer (NK) cells, plasma cells (antibody-secreting B cells), macrophages and granulocytes. Preferred effector cell surface molecules (“ECMs”) include CD2, CD3, CD8, CD16, TCR, and the NKG2D receptor. Accordingly, molecules capable of immunospecifically binding an epitope of such molecules, or to other effector cell surface molecules may be used in accordance with the principles of the present invention. Exemplary antibodies, whose VH and/or VL Domains, and/or 1, 2, or all 3 of the CDR_{L}s of the VL Region and/or 1, 2 or all 3 of the CDR_{H}s of the VH Domain, may be used to construct molecules capable of mediating the redirected killing of a target cell are provided below.

A. Exemplary CD2 Binding Capabilities

In one embodiment, the molecules of the invention are capable of binding to an epitope of CD2 present on the surface of such effector cell. CD2 is a cell adhesion molecule found on the surface of T-cells and natural killer (NK) cells. CD2 enhances NK cell cytotoxicity, possibly as a promoter of NK cell nanotube formation (Mace, E. M. et al. (2014) “Cell Biological Steps and Checkpoints in Accessing NK Cell Cytotoxicity,” Immunol. Cell. Biol. 92(3):245-255; Comerci, C. J. et al. (2012) “CD2 Promotes Human Natural Killer Cell Membrane Nanotube Formation,” PLoS One 7(10):e47664:1-12). Molecules that specifically bind CD2 include the anti-CD2 antibody “CD2 mAb Lo-CD2a.”

The amino acid sequence of the VH Domain of CD2 mAb Lo-CD2a (ATCC Accession No: HB-11423); SEQ ID NO:59) is shown below (CDR_{H} residues are shown underlined):

EVQLQQSGPE LQRPGASVKL SCKASGYIFT EYYMYWVKQR
PKQGLELVGR IDPEDGSIDY VEKFKKKATL TADTSSNTAY
MQLSSLTSED TATYFCARGK FNYRFAYWGQ GTLVTVSS

The amino acid sequence of the VL Domain of CD2 mAb Lo-CD2a (ATCC Accession No: 11423; SEQ ID NO:60) is shown below (CDR_{L} residues are shown underlined):

DVVLTQTPPT LLATIGQSVS ISCRSSQSLL HSSGNTYLNW
LLQRTGQSPQ PLIYLVSKLE SGVPNRFSGS GSGTDFTLKI
SGVEAEDLGV YYCMQFTHYP YTFGAGTKLE LK

B. Exemplary CD3 Binding Capabilities

In one embodiment, the molecules of the invention are capable of binding to an epitope of CD3. CD3 is a T-cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3c chains. These chains associate with a molecule known as the T-Cell Receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T-cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139). Molecules that specifically binds CD3 include the anti-CD3 antibodies “CD3 mAb 1” and “OKT3.” The anti-CD3 antibody CD3 mAb 1 is capable of binding non-human primates (e.g., cynomolgus monkey).

The amino acid sequence of the VH Domain of humanized CD3 mAb 1 (SEQ ID NO:61) is shown below (CDR_{H} residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The amino acid sequence of the VL Domain of humanized CD3 mAb 1 (SEQ ID NO:62) is shown below (CDR_{L} residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

A humanized variant “CD3 mAb 1 (D65G),” may be incorporated. CD3 mAb 1 (D65G) comprises the VL Domain of CD3 mAb 1 (SEQ ID NO:62) and a VH CD3 mAb 1 Domain having a D65G substitution (Kabat position 65, corresponding to residue 68 of SEQ ID NO:63).

The amino acid sequence of the VH of CD3 mAb 1 (D65G) (SEQ ID NO:63) is shown below (CDR_{H} residues are shown underlined, the substituted position (D65G) is shown in double underline):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL VTVSS

Alternatively, a humanized affinity variant of CD3 mAb 1 may be incorporated. Variants include a low affinity variant designated “CD3 mAb 1 Low” and a variant having a faster off rate designated “CD3 mAb 1 Fast.” The VL Domain of CD mAb 1 (SEQ ID NO:62) is common to CD3 mAb 1 Low and CD3 mAb1 Fast and is provided above. The amino acid sequences of the VH Domains of each of CD3 mAb 1 Low and CD3 mAb1 Fast are provided below.

The amino acid sequence of the Variable Heavy Chain Domain of anti-human CD3 mAb 1 Low (SEQ ID NO:64) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVT WFAYWGQGTL VTVSS

The amino acid sequence of the Variable Heavy Chain Domain of anti-human CD3 mAb 1 Fast (SEQ ID NO:65) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HKNFGNSYVT WFAYWGQGTL VTVSS

Another anti-CD3 antibody, which may be utilized is antibody Muromonab-CD3 “OKT3” (Xu et al. (2000) “In Vitro Characterization Of Five Humanized OKT3 Effector Function Variant Antibodies,” Cell. Immunol. 200:16-26); Norman, D. J. (1995) “Mechanisms Of Action And Overview Of OKT3,” Ther. Drug Monit. 17(6):615-620; Canafax, D. M. et al. (1987) “Monoclonal Antilymphocyte Antibody (OKT3) Treatment Of Acute Renal Allograft Rejection,” Pharmacotherapy 7(4):121-124; Swinnen, L. J. et al. (1993) “OKT3 Monoclonal Antibodies Induce Interleukin-6 And Interleukin-10: A Possible Cause Of Lymphoproliferative Disorders Associated With Transplantation,” Curr. Opin. Nephrol. Hypertens. 2(4):670-678).

The amino acid sequence of the VH Domain of OKT3 (SEQ ID NO:66) is shown below (CDR_{H} residues are shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSS

The amino acid sequence of the VL Domain of OKT3 (SEQ ID NO:67) is shown below (CDR_{L} residues are shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

Additional anti-CD3 antibodies that may be utilized include but are not limited to those described in PCT Publication Nos. WO 2008/119566; and WO 2005/118635.

C. Exemplary CD8 Binding Capabilities

In one embodiment, the molecules of the invention are capable of binding to an epitope of CD8 present on the surface of such effector cell. CD8 is a T-cell co-receptor composed of two distinct chains (Leahy, D. J., (1995) “A Structural View of CD4 and CD8,” FASEB J., 9:17-25) that is expressed on Cytotoxic T-cells. The activation of CD8⁺ T-cells has been found to be mediated through co-stimulatory interactions between an antigen:major histocompability class I (MHC I) molecule complex that is arrayed on the surface of a target cell and a complex of CD8 and the T-cell Receptor, that are arrayed on surface of the CD8⁺ T-cell (Gao, G., and Jakobsen, B., (2000). “Molecular interactions of coreceptor CD8 and MHC class I: the molecular basis for functional coordination with the T-Cell Receptor”. Immunol Today 21: 630-636). Unlike MHC II molecules, which are expressed by only certain immune system cells, MHC I molecules are very widely expressed. Thus, cytotoxic T-cells are capable of binding to a wide variety of cell types. Activated cytotoxic T-cells mediate cell killing through their release of the cytotoxins perforin, granzymes, and granulysin. Antibodies that specifically bind CD8 include the anti-CD8 antibodies “OKT8” and “TRX2.”

The amino acid sequence of the VH Domain of OKT8 (SEQ ID NO:68) is shown below (CDR_{H} residues are shown underlined):

QVQLLESGPE LLKPGASVKM SCKASGYTFT DYNMHWVKQS
HGKSLEWIGY IYPYTGGTGY NQKFKNKATL TVDSSSSTAY
MELRSLTSED SAVYYCARNF RYTYWYFDVW GQGTTVTVSS

The amino acid sequence of the VL Domain of OKT8 (SEQ ID NO:69) is shown below (CDR_{L} residues are shown underlined):

DIVMTQSPAS LAVSLGQRAT ISCRASESVD SYDNSLMHWY
QQKPGQPPKV LIYLASNLES GVPARFSGSG SRTDFTLTID
PVEADDAATY YCQQNNEDPY TFGGGTKLEI KR

The amino acid sequence of the VH Domain of TRX2 (SEQ ID NO:70) is shown below (CDR_{H} residues are shown underlined):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS S

The amino acid sequence of the VL Domain of TRX2 (SEQ ID NO:71) is shown below (CDR_{L} residues are shown underlined):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIK

D. Exemplary CD16 Binding Capabilities

In one embodiment, the molecules of the invention are capable of binding to an epitope of CD16. CD16 is expressed by neutrophils, eosinophils, natural killer (NK) cells, and tissue macrophages that bind aggregated but not monomeric human IgG (Peitz, G. A. et al. (1989) “Human Fc Gamma RIII: Cloning, Expression, And Identification Of The Chromosomal Locus Of Two Fc Receptors For IgG,” Proc. Natl. Acad. Sci. (U.S.A.) 86(3):1013-1017; Bachanova, V. et al. (2014) “NK Cells In Therapy Of Cancer,” Crit. Rev. Oncog. 19(1-2): 133-141; Miller, J. S. (2013) “Therapeutic Applications: Natural Killer Cells In The Clinic,” Hematology Am. Soc. Hematol. Educ. Program. 2013:247-253; Youinou, P. et al. (2002) “Pathogenic Effects Of Anti-Fc Gamma Receptor IIIB (CD16) On Polymorphonuclear Neutrophils In Non-Organ-Specific Autoimmune Diseases,” Autoimmun Rev. 1(1-2):13-19; Peipp, M. et al. (2002) “Bispecific Antibodies Targeting Cancer Cells,” Biochem. Soc. Trans. 30(4):507-511). Molecules that specifically bind CD16 include the anti-CD16 antibodies “3G8” and “A9.” Humanized 3G8 (“h3G8”) antibodies are described in PCT Publication WO 03/101485.

The amino acid sequence of the VH Domain of murine 3G8 (SEQ ID NO:72) is shown below (CDR_{H} residues are shown underlined):

QVTLKESGPG ILQPSQTLSL TCSFSGFSLR TSGMGVGWIR
QPSGKGLEWL AHIWWDDDKR YNPALKSRLT ISKDTSSNQV
FLKIASVDTA DTATYYCAQI NPAWFAYWGQ GTLVTVSA

The amino acid sequence of the VL Domain of murine 3G8 (SEQ ID NO:73) is shown below (CDR_{L} residues are shown underlined):

DTVLTQSPAS LAVSLGQRAT ISCKASQSVD FDGDSFMNWY
QQKPGQPPKL LIYTTSNLES GIPARFSASG SGTDFTLNIH
PVEEEDTATY YCQQSNEDPY TFGGGTKLEI K

The amino acid sequence of the VH Domain of h3G8 (SEQ ID NO:74) is shown below (CDR_{H} residues are shown underlined):

QVTLRESGPA LVKPTQTLTL TCTFSGFSLS TSGMGVGWIR
QPPGKALEWL AHIWWDDDKR YNPALKSRLT ISKDTSKNQV
VLTMTNMDPV DTATYYCAQI NPAWFAYWGQ GTLVTVSS

The amino acid sequence of the VL Domain of murine h3G8 (SEQ ID NO:75) is shown below (CDR_{L} residues are shown underlined):

DIVMTQSPDS LAVSLGERAT INCKASQSVD FDGDSFMNWY
QQKPGQPPKL LIYTTSNLES GVPDRFSGSG SGTDFTLTIS
SLQAEDVAVY YCQQSNEDPY TFGQGTKLEI K

The amino acid sequence of the VH Domain of A9 (SEQ ID NO:76) is shown below (CDR_{H} residues are shown underlined):

QVQLQQSGAE LVRPGTSVKI SCKASGYTFT NYWLGWVKQR
PGHGLEWIGD IYPGGGYTNY NEKFKGKATV TADTSSRTAY
VQVRSLTSED SAVYFCARSA SWYFDVWGAR TTVTVSS

The amino acid sequence of the VL Domain of A9 (SEQ ID NO:77) is shown below (CDR_{L} residues are shown underlined):

DIQAVVTQES ALTTSPGETV TLTCRSNTGT VTTSNYANWV
QEKPDHLFTG LIGHTNNRAP GVPARFSGSL IGDKAALTIT
GAQTEDEAIY FCALWYNNHW VTGGGTKLTVL

U.S. Pat. No. 9,035,026 describes an anti-CD16 antibody that is capable of binding to CD16A but not to CD16B. Such an antibody is particularly useful as a component of a bi- or multispecific binding molecule that is directed against disease-associated cells, as it would mainly recruit NK cells, and would not be bound by circulating soluble CD16B or diverted from NK cell binding by binding to neutrophils or activated eosinophils.

The amino acid sequence of the VH Domain of the anti-CD16A antibody of U.S. Pat. No. 9,035,026 (SEQ ID NO:78) is shown below (CDR_{H} residues are shown underlined):

EVQLVQSGAE VKKPGESLKV SCKASGYTFT SYYMHWVRQA
PGQGLEWMGI INPSGGSTSY AQKFQGRVTM TRDTSTSTVY
MELSSLRSED TAVYYCARGS AYYYDFADYW GQGTLVTVSS

The amino acid sequences of seven suitable VL Domain for such anti-CD16A antibody of U.S. Pat. No. 9,035,026 are shown below as SEQ ID NOs:79-85:

(SEQ ID NO: 79)
SYELMQPPSV SVSSGQTASI PCSGDKLEEK YVSWYQQRPG
QSPVLVIYQD NKRPSGIPER FSGSNSGNTA TLTISGTQAM
DEADYYCQVW DNYSVLFGGG TKLTVL
(SEQ ID NO: 80)
SYELTQPLSE SVAQGQTARI TCGGNNIESR NVHWYQQKPG
QAPVLVIYRD NNRPSGIPER FSGSNSGNMA TLTISRAQAG
DAADYYCQVW DNYTVLFGGG TKLTVL
(SEQ ID NO: 81)
SYELTQPPSV AVAPGKTARI TCGGNNIGSK NVHWYQQKPG
QAPVLVIYRD SNRPSGIPER FSGSNSGNTA TLTISRAQAG
DEADFYCQVW DNYIVLFGGG TKLTVL
(SEQ ID NO: 82)
QAVLTQPPSV SVAPGQTARI PCEGNNIGSK NVHWYRQKPG
QVPVLVMYDD SDRPSGIPER FSGSNSGNTA TLTISGTQAM
DEADYYCQVW DNYSVLFGGG TKLTVL
(SEQ ID NO: 83)
QPVLTQPLSV SVAPGQTARI TCGGNNIGSK NVHWYQQKPG
QAPVLVIYRD SSRPSGIPER LSGSNSGDTA TLTISRAQAG
DEADYYCQVW DDYIVVFGGG TKLTVL
(SEQ ID NO: 84)
SYELTQPPSV SVTPGQTATI TCGANDIGKR NVHWYQQRPG
QSPVLVIYQD NKRPSGIPER FSGSNSGNTA TLTISGTQAM
DEADYYCQVW DNYSVLFGGG TKLTVL
(SEQ ID NO: 85)
QPVLTQPSSV SVAPGQTATI SCGGHNIGSK NVHWYQQRPG
QSPVLVIYQD NKRPSGIPER FSGSNSGNTA TLTISGTQAM
DEADYYCQVW DNYSVLFGGG TKLTVL

In a further embodiment, the CD16-Binding Domains of humanized anti-CD16 antibody, hCD16-M1 or humanized anti-CD16 antibody, hCD16-M2,may be employed in concert with gp41-Binding Domains to produce multispecific Binding Molecules that are capable of binding CD16 and gp41. The amino acid sequence of the VH Domain of hCD16-M1 (SEQ ID NO:127) is shown below (CDR_{H} residues are shown underlined):

EVQLVESGGG LVKPGGSLRL SCAASGFTFS NYGMSWVRQA
PGKGLEWVAT ISGGGSYTFY PDSVKGRFTI SRDNAKNSLY
LQMNSLRTED TALYYCVRQS ARAPEPYWGQ GTLVTVSS

The amino acid sequence of the VL Domain of hCD16-M1 (SEQ ID NO:128) is shown below (CDR_{L} residues are shown underlined):

DIQMTQSPSF LSASVGDRVT ITCRASQNVG THVAWYQQKP
GKAPKSLLYS ASYRYSGVPS RFSGSGSGTD FTLTISSLQS
EDIATYYCQQ YKSYPLTFGQ GTKLEIK

Antibody hCD16-M2 is a humanized derivative of murine anti-human CD16 monoclonal antibody CD16-M2. Humanization resulted in two suitable VH Domains (hCD16-M2 VIII and hCD16-M2 VH2), either of which may be employed with the obtained humanized VL Domain (hCD16-M2 VL1).

The amino acid sequence of the VH Domain of hCD16-M2 VII1 (SEQ ID NO:129) is shown below (CDR_{H} residues are shown underlined):

QVQLVQSGAE VKKPGASVKV SCKASGYTFT SSAMHWVRQA
PGQGLEWMGY INHYNDGIKY NERFKGRVTI TADKSTSTAY
MELSSLRSED TAVYYCATGY RYASWFASWG QGTLVTVSS

The amino acid sequence of the VH Domain of hCD16-M2 VH2 (SEQ ID NO:130) is shown below (CDR_{H} residues are shown underlined):

QVQLVQSGAE VKKPGASVKV SCKASGYTFT SSAMHWVRQA PGQGLEWMGY

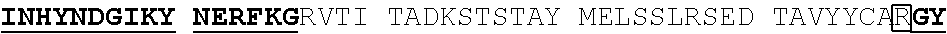

RYASWFASWG QGTLVTVSS

As will be recognized, the amino acid sequence of hCD16-M2 VH1 (SEQ ID NO:129) differs from that of hCD16-M2 VH2 (SEQ ID NO:130) in possessing a T98R substitution in the residue that immediately precedes CDR_{H}3 (shown boxed above).

The amino acid sequence of the VL Domain hCD16-M2 VL1 (SEQ ID NO:131) is shown below (CDR_{L} residues are shown underlined):

EIVLTQSPAT LSVSPGERAT LSCRASQNIG TSIHWYQQKP
DQSPKLLIKS VSESISGVPS RFSGSGSGTD FTLTINSLEA
EDFATYYCQQ SNSWPLTFGQ GTKLEIK

Additional anti-CD16 antibodies that may be utilized include but are not limited to commercially available antibodies DJ130c (Tamm, A. et al. (1996) “The Binding Epitopes Of Human CD16 (Fc Gamma Rill) Monoclonal Antibodies. Implications For Ligand Binding,” J. Immunol. 157(4):1576-1581), eBioCB16 (ThermoFisher) or 1D3 (Abcam), or those described in PCT Publication Nos. WO 03/101485; and WO 2006/125668.

E. Exemplary TCR Binding Capabilities

In one embodiment, the molecules of the invention are capable of binding to an epitope of the T Cell Receptor (TCR). The T Cell Receptor is natively expressed by CD4+ or CD8+ T cells, and permits such cells to recognize antigenic peptides that are bound and presented by class I or class II MHC proteins of antigen-presenting cells. Recognition of a pMHC (peptide—MHC) complex by a TCR initiates the propagation of a cellular immune response that leads to the production of cytokines and the lysis of the antigen-presenting cell (see, e.g., Armstrong, K. M. et al. (2008) “Conformational Changes And Flexibility In T-Cell Receptor Recognition Of Peptide—MHC Complexes,” Biochem. J. 415(Pt 2):183-196; Willemsen, R. (2008) “Selection Of Human Antibody Fragments Directed Against Tumor T-Cell Epitopes For Adoptive T-Cell Therapy,” Cytometry A. 73(11):1093-1099; Beier, K. C. et al. (2007) “Master Switches Of T-Cell Activation And Differentiation,” Eur. Respir. J. 29:804-812; Mallone, R. et al. (2005) “Targeting T Lymphocytes For Immune Monitoring And Intervention In Autoimmune Diabetes,” Am. J. Ther. 12(6):534-550). CD3 is the receptor that binds to the TCR (Thomas, S. et al. (2010) “Molecular Immunology Lessons From Therapeutic T-Cell Receptor Gene Transfer,” Immunology 129(2):170-177; Guy, C. S. et al. (2009) “Organization Of Proximal Signal Initiation At The TCR: CD3 Complex,” Immunol. Rev. 232(1):7-21; St. Clair, E.W. (Epub 2009 Oct. 12) “Novel Targeted Therapies For Autoimmunity,” Curr. Opin. Immunol. 21(6):648-657; Baeuerle, P. A. et al. (Epub 2009 Jun. 9) “Bispecific T-Cell Engaging Antibodies For Cancer Therapy,” Cancer Res. 69(12):4941-4944; Smith-Garvin, J. E. et al. (2009) “T Cell Activation,” Annu. Rev. Immunol. 27:591-619; Renders, L. et al. (2003) “Engineered CD3 Antibodies For Immunosuppression,” Clin. Exp. Immunol. 133(3):307-309).

Molecules that specifically bind to the T Cell Receptor include the anti-TCR antibody “BMA 031” (EP 0403156; Kurrle, R. et al. (1989) “BMA 031—A TCR-Specific Monoclonal Antibody For Clinical Application,” Transplant Proc. 21(1 Pt 1): 1017-1019; Nashan, B. et al. (1987) “Fine Specificity Of A Panel Of Antibodies Against The TCR/CD3 Complex,” Transplant Proc. 19(5):4270-4272; Shearman, C. W. et al. (1991) “Construction, Expression, And Biologic Activity Of Murine/Human Chimeric Antibodies With Specificity For The Human α/β T Cell,” J. Immunol. 146(3):928-935; Shearman, C. W. et al. (1991) “Construction, Expression And Characterization of Humanized Antibodies Directed Against The Human α/β T Cell Receptor,” J. Immunol. 147(12):4366-4373).

The amino acid sequence of a VH Domain of BMA 031 (SEQ ID NO:86) is shown below (CDR_{H} residues are shown underlined):

QVQLVQSGAE VKKPGASVKV SCKASGYKFT SYVMHWVRQA
PGQGLEWIGY INPYNDVTKY NEKFKGRVTI TADKSTSTAY
LQMNSLRSED TAVHYCARGS YYDYDGFVYW GQGTLVTVSS

The amino acid sequence of the VL Domain of BMA 031 (SEQ ID NO:87) is shown below (CDR_{L} residues are shown underlined):

EIVLTQSPAT LSLSPGERAT LSCSATSSVS YMHWYQQKPG
KAPKRWIYDT SKLASGVPSR FSGSGSGTEF TLTISSLQPE
DFATYYCQQW SSNPLTFGQG TKLEIK

F. Exemplary NKG2D Binding Capabilities

In one embodiment, the molecules of the invention are capable of binding to an epitope of the NKG2D receptor. The NKG2D receptor is expressed on all human (and other mammalian) Natural Killer cells (Bauer, S. et al. (1999) “Activation Of NK Cells And T Cells By NKG2D, A Receptor For Stress-Inducible MICA,” Science 285(5428):727-729; Jamieson, A. M. et al. (2002) “The Role Of The NKG2D Immunoreceptor In Immune Cell Activation And Natural Killing,” Immunity 17(1):19-29) as well as on all CD8⁺ T cells (Groh, V. et al. (2001) “Costimulation Of CD8α/β T Cells By NKG2D Via Engagement By MIC Induced On Virus-Infected Cells,” Nat. Immunol. 2(3):255-260; Jamieson, A. M. et al. (2002) “The Role Of The NKG2D Immunoreceptor In Immune Cell Activation And Natural Killing,” Immunity 17(1):19-29). Molecules that specifically bind to the NKG2D Receptor include the anti-NKG2D antibodies “KYK-1.0” and “KYK-2.0” (Kwong, K Y et al. (2008) “Generation, Affinity Maturation, And Characterization Of A Human Anti-Human NKG2D Monoclonal Antibody With Dual Antagonistic And Agonistic Activity,” J. Mol. Biol. 384:1143-1156).

The amino acid sequence of the VH Domain of KYK-1.0 (SEQ ID NO:88) is shown below (CDR_{H} residues are shown underlined):

EVQLVESGGG VVQPGGSLRL SCAASGFTFS SYGMHWVRQA
PGKGLEWVAF IRYDGSNKYY ADSVKGRFTI SRDNSKNTKY
LQMNSLRAED TAVYYCAKDR FGYYLDYWGQ GTLVTVSS

The amino acid sequence of the VL Domain of KYK-1.0 (SEQ ID NO:89) is shown below (CDR_{L} residues are shown underlined):

QPVLTQPSSV SVAPGETARI PCGGDDIETK SVHWYQQKPG
QAPVLVIYDD DDRPSGIPER FFGSNSGNTA TLSISRVEAG
DEADYYCQVW DDNNDEWVFG GGTQLTVL

The amino acid sequence of a VH Domain of KYK-2.0 (SEQ ID NO:90) is shown below (CDR_{H} residues are shown underlined):

QVQLVESGGG LVKPGGSLRL SCAASGFTFS SYGMHWVRQA
PGKGLEWVAF IRYDGSNKYY ADSVKGRFTI SRDNSKNTLY
LQMNSLRAED TAVYYCAKDR GLGDGTYFDY WGQGTTVTVS S

The amino acid sequence of a VL Domain of KYK-2.0 (SEQ ID NO:91) is shown below (CDR_{L} residues are shown underlined):

QSALTQPASV SGSPGQSITI SCSGSSSNIG NNAVNWYQQL
PGKAPKLLIY YDDLLPSGVS DRFSGSKSGT SAFLAISGLQ
SEDEADYYCA AWDDSLNGPV FGGGTKLTVL

G. Exemplary NKp46 Binding Capabilities

In one embodiment, the molecules of the invention are capable of binding to an epitope of NKp46 (CD335). NKp46 is a major NK cell-activating receptor that is involved in the elimination of target cells (Sivori S, et al. (1997). “p46, a Novel Natural Killer Cell—specific Surface Molecule That Mediates Cell Activation,” J. Exp. Med. 186 (7): 1129-36.). NKp46 is uniquely expressed on all NK cell subsets (Narni-Mancinelli E, et al. (2011). “Fate mapping analysis of lymphoid cells expressing the NKp46 cell surface receptor,” Proc Natl Acad Sci USA 108:18324-9). Molecules that specifically bind to NKp46 include the anti-NKp46 antibodies “BAB281” and “NKp46-3” (WO 2015/197593).

The amino acid sequence of the VH Domain of BAB281 (SEQ ID NO:92) is shown below (CDR_{H} residues are shown underlined):

QIQLVQSGPE LQKPGETVKI SCKASGYTFT NYGMNWVKQA
PGKGLKWMGW INTNTGEPTY AEEFKGRFAF SLETSASTAY
LQINNLKNED TATYFCARDY LYYFDYWGQG TTLTVSS

The amino acid sequence of the VL Domain of BAB281 (SEQ ID NO:93) is shown below (CDR_{L} residues are shown underlined):

DNIVMTQSPK SMSMSVGERV TLTCKASENV VTYVSWYQQK
PEQSPKLLIY GASNRYTGVP DRFTGSGSAT DFTLTISSVQ
AEDLADYHCG QGYSYPYTFG GGTKLEIK

The amino acid sequence of the VH Domain of NKp46-3 (SEQ ID NO:94) is shown below (CDR_{H} residues are shown underlined):

EVQLQQSGPE LVKPGASVKI SCKTSGYTFT EYTMHWVKQS
HGKSLEWIGG ISPNIGGTSY NQKFKGKATL TVDKSSSTAY
MELRSLTSED SAVYYCARRG GSFDYWGQGT TLTVSS

The amino acid sequence of the VL Domain of NKp46-3 (SEQ ID NO:95) is shown below (CDR_{L} residues are shown underlined):

DIVMTQSPAT LSVTPGDRVS LSCRASQSIS DYLHWYQQKS
HESPRLLIKY ASQSISGIPS RFSGSGSGSD FTLSINSVEP
EDVGVYYCQN GHSFPLTFGA GTKLELK

Additional antibodies that bind NKp46 are described in WO 2015/197593 and WO 2017/016805. Other exemplary antibodies that bind to the cell surface of a Natural Killer cell include antibodies: Al, AC2, EPR3678(2), EPR20461, EPR20627 and IMG17B5F11 (which bind CD39); TB01, HNK-1/Leu-7 and NK1 (which bind CD57); FN50 (which binds CD69); 5B5, B-L2, TS82b and C33 (which bind CD82); 3B3, B199.2 and EP7169 (which bind CD161); 17D9 (which binds CLEC1B); 2F9 (which binds KIR2DL1); EPR8825 (which binds KIR2DL2); mAb 33 (which binds KIR2DL4); 11E3, 17B4, EPR4392(2), EPR20261 and EPR 20627 (which bind Lymphocyte Activation Gene 3); A10, C7, CX5, 1D11 and MM0489-10R27 (which bind NKG2D); BMK13 (which binds PRG2); EPR9916 (which binds SLAMF6); etc. Antibodies capable of binding to each of such NK cell surface molecules are commercially available from Abcam plc and other sources, and may be readily adapted to the purposes of the present invention.

### VI. Exemplary Alternative HIV-1 Binding Molecules

The mature human immunodeficiency virus type 1 (HIV-1) envelope (Env) glycoprotein trimer is comprised of three copies of a noncovalently linked gp120/gp41 heterodimer that arises from cleavage of the viral gp160 precursor protein. In one embodiment, the molecules of the invention are capable of binding to an epitope of the HIV-1 Env protein gp120, gp160 and/or gp41 that is distinct from the epitope of 7B2.

Monoclonal antibody A32 recognizes a conformational epitope in the C1 region of HIV-1 Env gp120 (Wyatt et al. (1995) “Involvement Of The V1/V2 Variable Loop Structure In The Exposure Of Human Immunodeficiency Virus Type 1 gp120 Epitopes Induced By Receptor Binding,” J. Virol. 69:5723-5733) and mediates potent ADCC activity and could block a significant proportion of ADCC-mediating Ab activity detectable in HIV-1 infected individuals (Ferrari, G. et al. (2011) “An HIV-1 gp120 Envelope Human Monoclonal Antibody That Recognizes a CI Conformational Epitope Mediates Potent Antibody-Dependent Cellular Cytotoxicity (ADCC) Activity and Defines a Common ADCC Epitope in Human HIV-1 Serum,” J. Virol. 85(14):7029-7036).

Multiple VH Domains of Antibody A32 have been reported in the art that possess minor changes in framework regions 1 and/or 4 reported (see, e.g., Protein Data Base Accession number PDB: 4YBL_H, US 2015/0239961 and WO 2006/044410). Any of these variant Antibody A32 VH Domains may be employed in accordance with the present invention. The amino acid sequence of an illustrative VH Domain of A32 (SEQ ID NO:96) is shown below (CDR_{H} residues are shown underlined):

QVQLQESGPG LVKPSQTLSL SCTVSGGSSS SGAHYWSWIR
QYPGKGLEWI GYIHYSGNTY YNPSLKSRIT ISQHTSENQF
SLKLNSVTVA DTAVYYCARG TRLRTLRNAF DIWGQGTLVT
VSS

The amino acid sequence of the VL Domain of A32 (SEQ ID NO:97) is shown below (CDR_{L} residues are shown underlined):

QSALTQPPSA SGSPGQSVTI SCTGTSSDVG GYNYVSWYQH
HPGKAPKLII SEVNNRPSGV PDRFSGSKSG NTASLTVSGL
QAEDEAEYYC SSYTDIHNFV FGGGTKLTVL

The amino acid sequence of the VL Domain of A32 (SEQ ID NO:97) may be employed with the illustrative VH Domain of A32 (SEQ ID NO:96) or with any of the variant Antibody A32 VH Domains (see, e.g., Protein Data Base Accession number PDB: 4YBL_H, US 2015/0239961 and WO 2006/044410) to form an anti-HIV-1 Env gp120 Epitope Binding Site.

Monoclonal antibody 10-1074 targets the base of the V3 loop of HIV-1 Env gp120 (see, e.g., WO 2014/063059) and is among the most potent anti-HIV-1 neutralizing antibodies isolated and has shown some in-vivo activity in an early stage clinical trial (Caskey, M., et al., (2017) “Antibody 10-1074 Suppresses Viremia In HIV-1-infected individuals.” Nat Med. 23:185-191).

The amino acid sequence of the VH Domain of 10-1074 (SEQ ID NO:98) is shown below (CDR_{H} residues are shown underlined):

QVQLQESGPG LVKPSETLSV TCSVSGDSMN NYYWTWIRQS
PGKGLEWIGY ISDRESATYN PSLNSRVVIS RDTSKNQLSL
KLNSVTPADT AVYYCATARR GQRIYGVVSF GEFFYYYSMD
VWGKGTTVTV SS

The amino acid sequence of the VL Domain of 10-1074 (SEQ ID NO:99) is shown below (CDR_{L} residues are shown underlined):

SYVRPLSVAL GETARISCGR QALGSRAVQW YQHRPGQAPI
LLIYNNQDRP SGIPERFSGT PDINFGTRAT LTISGVEAGD
EADYYCHMWD SRSGFSWSFG GATRLTVL

Monoclonal antibody 3BNC117 targets the CD4 binding site of gp120 and is a broadly neutralizing anti-HIV-1 antibody (see, e.g., WO 2013/016468) that has shown some in-vivo activity in an early stage clinical trial (Caskey, M., et al. (2015) “Viraemia Suppressed In HIV-1-Infected Humans By Broadly Neutralizing Antibody 3BNC117,” Nature 522:487-491).

The amino acid sequence of the VH Domain of 3BNC117 (SEQ ID NO:100) is shown below (CDR_{H} residues are shown underlined):

QVQLLQSGAA VTKPGASVRV SCEASGYNIR DYFIHWWRQA
PGQGLQWVGW INPKTGQPNN PRQFQGRVSL TRHASWDFDT
FSFYMDLKAL RSDDTAVYFC ARQRSDYWDF DVWGSGTQVT
VSS

The amino acid sequence of the VL Domain of 3BNC117 (SEQ ID NO:101) is shown below (CDR_{L} residues are shown underlined):

DIQMTQSPSS LSASVGDTVT ITCQANGYLN WYQQRRGKAP
KLLIYDGSKL ERGVPSRFSG RRWGQEYNLT INNLQPEDIA
TYFCQVYEFV VPGTRLDLK

Monoclonal antibodies PGT121 and PGT145 are broadly neutralizing HIV-1 antibodies that are largely dependent on the gp120 glycan for Env recognition (Mouquet H, et al., (2012) “Complex-type N-glycan recognition by potent broadly neutralizing HIV antibodies.” Proc Natl Acad Sci USA 109: E3268-3277; Yasmeen, A., et al. (2014) “Differential Binding Of Neutralizing And Non-Neutralizing Antibodies To Native-Like Soluble HIV-1 Env Trimers, Uncleaved Env Proteins, And Monomeric Subunits.” Retrovirology 11:41; WO 2012/030904).

The amino acid sequence of the VH Domain of PGT121 (SEQ ID NO:102) is shown below (CDR_{H} residues are shown underlined):

QMQLQESGPG LVKPSETLSL TCSVSGASIS DSYWSWIRRS
PGKGLEWIGY VHKSGDTNYS PSLKSRVNLS LDTSKNQVSL
SLVAATAADS GKYYCARTLH GRRIYGIVAF NEWFTYFYMD
VWGNGTQVTVSS

The amino acid sequence of the VL Domain of PGT121 (SEQ ID NO:103) is shown below (CDR_{L} residues are shown underlined):

SDISVAPGET ARISCGEKSL GSRAVQWYQH RAGQAPSLII
YNNQDRPSGI PERFSGSPDS PFGTTATLTI TSVEAGDEAD
YYCHIWDSRV PTKWVFGGGT TLTVL

The amino acid sequence of the VH Domain of PGT145 (SEQ ID NO:104) is shown below (CDR_{H} residues are shown underlined):

QVQLVQSGAE VKKPGSSVKV SCKASGNSFS NHDVHWVRQA
TGQGLEWMGW MSHEGDKTGL AQKFQGRVTI TRDSGASTVY
MELRGLTADD TAIYYCLTGS KHRLRDYFLY NEYGPNYEEW
GDYLATLDVW GHGTAVTVSS

The amino acid sequence of the VL Domain of PGT145 (SEQ ID NO:105) is shown below (CDR_{L} residues are shown underlined):

EVVITQSPLF LPVTPGEAAS LSCKCSHSLQ HSTGANYLAW
YLQRPGQTPR LLIHLATHRA SGVPDRFSGS GSGTDFTLKI
SRVESDDVGT YYCMQGLHSP WTFGQGTKVE IK

Monoclonal antibody VRC01 is a broadly neutralizing anti-HIV-1 antibody directed against the CD4 binding site of gp120 (Wu, X. et al. (2010) “Rational Design Of Envelope Identifies Broadly Neutralizing Human Monoclonal Antibodies To HIV-1,” Science 329:856-861 and Zhou, T. et al. (2010) “Structural Basis For Broad And Potent Neutralization Of HIV-1 By Antibody VRC01,” Science 329:811-817; WO 2013/163427).

The amino acid sequence of the VH Domain of VRC01 (SEQ ID NO:106) is shown below (CDR_{H} residues are shown underlined):

QVQLVQSGGQ MKKPGESMRI SCRASGYEFI DCTLNWIRLA
PGKRPEWMGW LKPRGGAVNY ARPLQGRVTM TRDVYSDTAF
LELRSLTVDD TAVYFCTRGK NCDYNWDFEH WGRGTPVIVS
S

The amino acid sequence of the VL Domain of VRC01 (SEQ ID NO:107) is shown below (CDR_{L} residues are shown underlined):

EIVLTQSPGT LSLSPGETAI ISCRTSQYGS LAWYQQRPGQ
APRLVIYSGS TRAAGIPDRF SGSRWGPDYN LTISNLESGD
FGVYYCQQYE FFGQGTKVQV DIKR

Monoclonal antibody 10E8 is a broadly neutralizing HIV-1 gp41 membrane-proximal external region (MPER)-specific antibody (Huang, J., et al. 2012. “Broad And Potent Neutralization Of HIV-1 By A gp41-Specific Human Antibody.” Nature. 491:406-12).

The amino acid sequence of the VH Domain of 10E8 (SEQ ID NO:108) is shown below (CDR_{H} residues are shown underlined):

EVQLVESGGG LVKPGGSLRL SCSASGFDFD NAWMTWVRQP
PGKGLEWVGR ITGPGEGWSV DYAAPVEGRF TISRLNSINF
LYLEMNNLRM EDSGLYFCAR TGKYYDFWSG YPPGEEYFQD
WGRGTLVTVS S

The amino acid sequence of the VL Domain of 10E8 (SEQ ID NO:109) is shown below (CDR_{L} residues are shown underlined):

SYELTQETGV SVALGRTVTI TCRGDSLRSH YASWYQKKPG
QAPILLFYGK NNRPSGVPDR FSGSASGNRA SLTISGAQAE
DDAEYYCSSR DKSGSRLSVF GGGTKLTVL

The present invention specifically includes and encompasses multispecific gp41-Binding Molecules that comprise the VL and/or VH Domain, and/or 1, 2 or all 3 of the CDRLs of the VL Region and/or 1, 2 or all 3 of the CDRHs of the VH Domain of the anti-HIV-1 monoclonal antibodies provided above.

### VII. Exemplary Multispecific gp41-Binding Molecules

Provided herein are a series of exemplary gp41-Binding Molecules incorporating a First Epitope Binding Domain that is immunospecific for an epitope of gp41 (i.e., a gp41-Binding Domain) and a Second Epitope Binding Domain that is immunospecific for an epitope of an ECM. Optionally such molecules incorporate a Third Epitope Binding Domain (or a Third Epitope Binding Domain and a Fourth Epitope Binding Domain) that is immunospecific for a different epitope of gp41 and/or an epitope of a different HIV-1 molecule and/or a different epitope of the ECM and/or an epitope of a different ECM. Particularly preferred are such molecules that comprise the optimized gp41-binding domains of 7B2GL. The structures and sequences of such illustrative gp41-Binding Molecules are summarized in Table 7 and are described in detail below. As will be recognized, analogous molecules may likewise be constructed (by employing the VL and VH domains of desired antibodies in lieu of the VL and VH domains used in the illustrative gp41-Binding Molecules.

TABLE 7

| | | | VL Domain | | VH Domain | | | |
|---|---|---|---|---|---|---|---|---|
| Diabody | Polypeptide | SEQ | | SEQ | | SEQ | Other | SEQ |
| Name | Chain | ID NO | Antibody | ID NO | Antibody | ID NO | Domain | ID NO |
| DART-1 | 1 | 110 | 7B2 | 55 | CD3 | 63 | Cys-E-Coil | 31 |
| | | | | | mAb 1 | | Knob-Bearing | 48 |
| | | | | | (G65D) | | Fc | |
| | 2 | 111 | CD3 mAb 1 | 62 | 7B2 | 56 | Cys-K-Coil | 32 |
| | 3 | 112 | Common Diabody Polypeptide Chain | | | | Hole-Bearing | 50 |
| | | | | | | | Fc | |
| DART-A | 1 | 113 | 7B2GL | 57 | CD3 | 63 | Cys-E-Coil | 31 |
| | | | | | mAb 1 | | Knob-Bearing | 48 |
| | | | | | (G65D) | | Fc | |
| | 2 | 114 | CD3 mAb 1 | 62 | 7B2GL | 58 | Cys-K-Coil | 32 |
| | 3 | 112 | Common Diabody Polypeptide Chain | | | | Hole-Bearing | 50 |
| | | | | | | | Fc | |
| DART-B | 1 | 115 | 7B2GL | 57 | h3G8 | 74 | Cys-E-Coil | 31 |
| | | | | | | | Knob-Bearing | 48 |
| | | | | | | | Fc | |
| | 2 | 116 | h3G8 | 75 | 7B2GL | 58 | Cys-K-Coil | 32 |
| | 3 | 112 | Common Diabody Polypeptide Chain | | | | Hole-Bearing | 50 |
| | | | | | | | Fc | |
| TRIDENT- | 1 | 113 | 7B2GL | 57 | CD3 | 63 | Cys-E-Coil | 31 |
| A | | | | | mAb 1 | | Knob-Bearing | 48 |
| | | | | | (G65D) | | Fc | |
| | 2 | 114 | CD3 mAb 1 | 62 | 7B2GL | 58 | Cys-K-Coil | 32 |
| | 3 | 117 | | | TRX2 | 70 | IgG CH1 | 3 |
| | | | | | | | IgG Hinge | 7 |
| | | | | | | | Hole-Bearing | 50 |
| | | | | | | | Fc | |
| | 4 | 118 | TRX2 | 71 | | | CL Kappa | 1 |
| TRIDENT- | 1 | 113 | 7B2GL | 57 | CD3 | 63 | Cys-E-Coil | 31 |
| B | | | | | mAb 1 | | Knob-Bearing | 48 |
| | | | | | (G65D) | | Fc | |
| | 2 | 114 | CD3 mAb 1 | 62 | 7B2GL | 58 | Cys-K-Coil | 32 |
| | 3 | 119 | | | A32 | 96 | IgG CH1 | 3 |
| | | | | | | | IgG Hinge | 7 |
| | | | | | | | Hole-Bearing | 50 |
| | | | | | | | Fc | |
| | 4 | 120 | A32 | 97 | | | CL Kappa | 1 |
| TRIDENT- | 1 | 115 | 7B2GL | 57 | h3G8 | 74 | Cys-E-Coil | 31 |
| C | | | | | | | Knob-Bearing | 48 |
| | | | | | | | Fc | |
| | 2 | 116 | h3G8 | 75 | 7B2GL | 58 | Cys-K-Coil | 32 |
| | 3 | 119 | | | A32 | 96 | IgG CH1 | 3 |
| | | | | | | | IgG Hinge | 7 |
| | | | | | | | Hole-Bearing | 50 |
| | | | | | | | Fc | |
| | 4 | 120 | A32 | 97 | | | CL Kappa | 1 |
| TRIDENT- | 1 | 113 | 7B2GL | 57 | CD3 | 63 | Cys-E-Coil | 31 |
| D | | | | | mAb 1 | | Knob-Bearing | 48 |
| | | | | | (G65D) | | Fc Domain | |
| | 2 | 114 | CD3 mAb 1 | 62 | 7B2GL | 58 | Cys-K-Coil | 32 |
| | 3 | 121 | | | 7B2GL | 58 | IgG CH1 | 3 |
| | | | | | | | IgG Hinge | 7 |
| | | | | | | | Hole-Bearing | 50 |
| | | | | | | | Fc | |
| | 4 | 122 | 7B2GL | 57 | | | CL Kappa | 1 |

A. gp41×CD3 Binding Molecule, DART-1

The gp41×CD3 Binding Molecule designated “DART-1” is a first illustrative bispecific gp41-Binding Molecule. DART-1 is an Fc Domain-containing, bispecific diabody capable of binding gp41 and the CD3 antigen. DART-1 is composed of three polypeptide chains and possesses one Binding Domain that comprises the VL and VH Domains of the anti-human gp41 antibody 7B2 (and is thus immunospecific for an epitope of gp41) and one Binding Domain that comprises the VL and VH Domains of CD3 mAb 1 (and is thus immunospecific for an epitope of the CD3 Antigen). The three polypeptide chains associate to form a covalently bonded diabody capable of immunospecifically binding the epitope of gp41 and the epitope of the CD3 Antigen (see, e.g., FIG. 4A).

The first polypeptide chain of DART-1 has the amino acid sequence of SEQ ID NO:110:

DIVMTQSPDS LAVSPGERAT IHCKSSQTLL YSSNNRHSIA
WYQQRPGQPP KLLLYWASMR LSGVPDRFSG SGSGTDFTLT
INNLQAEDVA IYYCHQYSSH PPTFGHGTRV EIKGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

Residues 1-113 of the first polypeptide chain (SEQ ID NO:110) of DART-1 correspond to the VL Domain of 7B2 (SEQ ID NO:55). Residues 114-121 (double underlined) of the first polypeptide chain correspond to Linker 1 (GGGSGGGG; SEQ ID NO:16). Residues 122-246 of the first polypeptide chain correspond to the VH Domain of CD3 mAb 1 (D65G) (SEQ ID NO:63). Residues 247-251 correspond to a linker (SEQ ID NO:21, underlined). Residues 252-279 of the first polypeptide chain correspond to a cysteine-containing E-coil (SEQ ID NO:31). Residues 280-292 of the first polypeptide chain correspond to a linker (SEQ ID NO:40). Residues 293-509 of the first polypeptide chain correspond to a “knob-bearing” (SEQ ID NO:48), in which the final residue is lysine.

The second polypeptide chain of DART-1 has the amino acid sequence of SEQ ID NO:111:

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGGGVF KPGGSLRLSC EASGFTFTEY YMTWVRQAPG
KGLEWLAYIS KNGEYSKYSP SSNGRFTISR DNAKNSVFLQ
LDRLSADDTA VYYCARADGL TYFSELLQYI FDLWGQGARV
TVSSASTKGK VAACKEKVAA LKEKVAALKE KVAALKE

Residues 1-110 of the second polypeptide chain (SEQ ID NO:111) of DART-1 correspond to the VL Domain of CD3 mAb 1 (SEQ ID NO:62). Residues 111-118 (double underlined) of the second polypeptide chain correspond to Linker 1 (GGGSGGGG; SEQ ID NO:16). Residues 119-244 of the second polypeptide chain correspond to the VH Domain of 7B2 (SEQ ID NO:56). Residues 245-249 of the second polypeptide chain correspond to a linker (SEQ ID NO:21, underlined). Residues 250-277 of the second polypeptide chain correspond to a cysteine-containing K-coil (SEQ ID NO:32).

The third polypeptide chain of the DART-1 has the amino acid sequence of SEQ ID NO:112:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

Residues 1-10 of the third polypeptide chain (SEQ ID NO:112) of DART-1 correspond to a linker (SEQ ID NO:40). Residues 11-227 of the third polypeptide chain correspond to a “hole-bearing” IgG1 CH2-CH3 Domain (SEQ ID NO:50), containing the H435R substitution (shown underlined), and in which the final residue is lysine. As stated above, the H435R substitution eliminates the ability of the molecule to bind to bind Protein A.

As will be recognized, the third polypeptide chain of DART-1 does not contain any Epitope Binding sites and may thus be employed in various gp41-Binding Molecules have the general structure provided in FIGS. 4A-4B. Accordingly, the third polypeptide chain of DART-1 is referred to herein as the “Common Diabody Polypeptide Chain.”

B. gp41×CD3 Binding Molecule, DART-A

A second illustrative gp41×CD3 Binding Molecule, designated “DART-A,” is similar to the above-described DART-1, but contains the VL and VH Domains of anti-human gp41 antibody 7B2GL in lieu of the parental 7B2 VL and VH Domains.

The first polypeptide chain of DART-A has the amino acid sequence of SEQ ID NO:113:

DIVMTQSPDS LAVSLGERAT INCKSSQTLL YSSNNRHSIA
WYQQKPGQPP KLLIYWASMR LSGVPDRFSG SGSGTDFTLT
ISSLQAEDVA VYYCHQYSSH PPTFGQGTKV EIKGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

Residues 1-113 of the first polypeptide chain (SEQ ID NO:113) of DART-A correspond to the VL Domain of 7B2GL (SEQ ID NO:57). Residues 114-121 (double underlined) of the first polypeptide chain correspond to Linker 1 (GGGSGGGG; SEQ ID NO:16). Residues 122-246 of the first polypeptide chain correspond to the VH Domain of CD3 mAb 1 (D65G) (SEQ ID NO:63). Residues 247-251 correspond to a linker (SEQ ID NO:21, underlined). Residues 252-279 of the first polypeptide chain correspond to a cysteine-containing E-coil (SEQ ID NO:31). Residues 280-292 of the first polypeptide chain correspond to a linker (SEQ ID NO:40). Residues 293-509 of the first polypeptide chain correspond to a “knob-bearing” (SEQ ID NO:48), in which the final residue is lysine.

The second polypeptide chain of DART-A has the amino acid sequence of SEQ ID NO:114:

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVESGGGLV KPGGSLRLSC AASGFTFSEY YMTWIRQAPG
KGLEWVSYIS KNGEYSKYSP SSNGRFTISR DNAKNSLYLQ
MNSLRAEDTA VYYCARADGL TYFSELLQYI FDLWGQGTLV
TVSSASTKGK VAACKEKVAA LKEKVAALKE KVAALKE

Residues 1-110 of the second polypeptide chain (SEQ ID NO:114) of DART-A correspond to the VL Domain of CD3 mAb 1 (SEQ ID NO:62). Residues 111-118 (double underlined) of the second polypeptide chain correspond to Linker 1 (GGGSGGGG; SEQ ID NO:16). Residues 119-244 of the second polypeptide chain correspond to the VH Domain of 7B2GL (SEQ ID NO:58). Residues 245-249 of the second polypeptide chain correspond to a linker (SEQ ID NO:21, underlined). Residues 250-277 of the second polypeptide chain correspond to a cysteine-containing K-coil (SEQ ID NO:32).

The amino acid sequence of the third polypeptide chain of DART-A is the same as that of the third polypeptide chain of DART-1 (i.e., SEQ ID NO:112).

C. gp41×CD16 Binding Molecule, DART-B

The gp41×CD16 Binding Molecule designated “DART-B” is a third illustrative bispecific gp41-Binding Molecule. DART-B is similar to the above-described DART-A, but comprises a CD16-Binding Domain in lieu of the CD3-Binding Domain of DART-A. Thus, DART-B is composed of three polypeptide chains and possesses one Binding Domain that comprises the VL and VH Domains of the anti-human gp41 antibody 7B2GL (and is thus immunospecific for an epitope of gp41), and one Binding Domain that comprises the VL and VH Domains of the anti-human CD16 antibody 3G8 (and thus is immunospecific for an epitope of CD16).

The first polypeptide chain of DART-B has the amino acid sequence of SEQ ID NO:115:

DIVMTQSPDS LAVSLGERAT INCKSSQTLL YSSNNRHSIA
WYQQKPGQPP KLLIYWASMR LSGVPDRFSG SGSGTDFTLT
ISSLQAEDVA VYYCHQYSSH PPTFGQGTKV EIKGGGSGGG
GQVTLRESGP ALVKPTQTLT LTCTFSGFSL STSGMGVGWI
RQPPGKALEW LAHIWWDDDK RYNPALKSRL TISKDTSKNQ
VVLTMTNMDP VDTATYYCAQ INPAWFAYWG QGTLVTVSSA
STKGEVAACE KEVAALEKEV AALEKEVAAL EKGGGDKTHT
CPPCPAPEAA GGPSVFLFPP KPKDTLMISR TPEVTCVVVD
VSHEDPEVKF NWYVDGVEVH NAKTKPREEQ YNSTYRVVSV
LTVLHQDWLN GKEYKCKVSN KALPAPIEKT ISKAKGQPRE
PQVYTLPPSR EEMTKNQVSL WCLVKGFYPS DIAVEWESNG
QPENNYKTTP PVLDSDGSFF LYSKLTVDKS RWQQGNVFSC
SVMHEALHNH YTQKSLSLSP GK

Residues 1-113 of the first polypeptide chain (SEQ ID NO:115) of DART-B correspond to the VL Domain of 7B2GL (SEQ ID NO:57). Residues 114-121 (double underlined) of the first polypeptide chain correspond to Linker 1 (GGGSGGGG; SEQ ID NO:16). Residues 122-239 of the first polypeptide chain correspond to the VH Domain of h3G8 (SEQ ID NO:74). Residues 240-244 correspond to a linker (SEQ ID NO:21, underlined). Residues 245-272 of the first polypeptide chain correspond to a cysteine-containing E-coil (SEQ ID NO:31). Residues 273-285 of the first polypeptide chain correspond to a linker (SEQ ID NO:40). Residues 286-502 of the first polypeptide chain correspond to a “knob-bearing” (SEQ ID NO:48), in which the final residue is lysine.

The second polypeptide chain of DART-B has the amino acid sequence of SEQ ID NO:116:

DIVMTQSPDS LAVSLGERAT INCKASQSVD FDGDSFMNWY
QQKPGQPPKL LIYTTSNLES GVPDRFSGSG SGTDFTLTIS
SLQAEDVAVY YCQQSNEDPY TFGQGTKLEI KGGGSGGGGQ
VQLVESGGGL VKPGGSLRLS CAASGFTFSE YYMTWIRQAP
GKGLEWVSYI SKNGEYSKYS PSSNGRFTIS RDNAKNSLYL
QMNSLRAEDT AVYYCARADG LTYFSELLQY IFDLWGQGTL
VTVSSASTKG KVAACKEKVA ALKEKVAALK EKVAALKE

Residues 1-111 of the second polypeptide chain (SEQ ID NO:116) of DART-B correspond to the VL Domain of h3G8 (SEQ ID NO:75). Residues 112-119 (double underlined) of the second polypeptide chain correspond to Linker 1 (GGGSGGGG; SEQ ID NO:16). Residues 120-245 of the second polypeptide chain correspond to the VH Domain of 7B2GL (SEQ ID NO:58). Residues 246-250 of the second polypeptide chain correspond to a linker (SEQ ID NO:21, underlined). Residues 251-278 of the second polypeptide chain correspond to a cysteine-containing K-coil (SEQ ID NO:32).

The amino acid sequence of the third polypeptide chain of DART-B is the same as that of the third polypeptide chain of DART-1 (i.e., SEQ ID NO:112).

D. gp41×CD3×CD8 Binding Molecule, TRIDENT-A

The gp41×CD3×CD8 Binding Molecule designated “TRIDENT-A” is a first illustrative trivalent gp41-Binding Molecule. TRIDENT-A is composed of four polypeptide chains and possesses one Binding Domain that comprises the VL and VH Domains of the anti-human gp41 antibody 7B2GL (and is thus immunospecific for an epitope of gp41), one Binding Domain that comprises the VL and VH Domains of CD3 mAb 1 (and is thus immunospecific for an epitope of the CD3 Antigen) and one Binding Domain that comprises the VL and VH Domains of TRX8 (and is thus immunospecific for an epitope of the CD8 Antigen). The four polypeptide chains associate to form a covalently bonded trivalent molecule capable of immunospecifically binding the epitope of gp41, the epitope of the CD3 Antigen and the epitope of CD8 (see, e.g., FIG. 6A).

The amino acid sequence of the first polypeptide chain of TRIDENT-A is the same as that of the first polypeptide chain of the above-described DART-A diabody (SEQ ID NO:113). Similarly, the amino acid sequence of the second polypeptide chain of TRIDENT-A is the same as that of the second polypeptide chain of the above-described DART-A diabody (SEQ ID NO:114).

The third polypeptide chain of TRIDENT-A has the amino acid sequence of SEQ ID NO:117:

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

Residues 1-121 of the third polypeptide chain of TRIDENT-A correspond to the VH Domain of the anti-CD8 antibody TRX2 (SEQ ID NO:70). Residues 121-219 correspond to an IgG1 CH1 Domain (SEQ ID NO:3). Residues 220-234 correspond to an IgG1 Hinge Domain (SEQ ID NO:7). Residues 235-451 correspond to the IgG1 “hole-bearing” CH2-CH3 Domain (SEQ ID NO:50).

The fourth polypeptide chain of TRIDENT-A has the amino acid sequence of SEQ ID NO:118:

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

Residues 1-106 of the fourth polypeptide chain of TRIDENT-A correspond to the VL Domain of the anti-CD8 antibody TRX2 (SEQ ID NO:71). Residues 107-213 correspond to a CL Kappa Domain (SEQ ID NO:1).

E. gp41×CD3×gp120 Binding Molecule, TRIDENT-B

A second illustrative trivalent gp41×CD3×gp120 Binding Molecule, designated “TRIDENT-B,” is similar to the above-described TRIDENT-A, but contains the VL and VH Domains of anti-human gp120 antibody A32 in lieu of the TRX8 VL and VH Domains of TRIDENT-A. Thus, TRIDENT-B is composed of four polypeptide chains and possesses one Binding Domain that comprises the VL and VH Domains of the anti-human gp41 antibody 7B2GL (and is thus immunospecific for an epitope of gp41), one Binding Domain that comprises the VL and VH Domains of CD3 mAb 1 (and is thus immunospecific for an epitope of the CD3 Antigen) and one Binding Domain that comprises the VL and VH Domains of A32 (and is thus immunospecific for an epitope of the gp120 Antigen).

The amino acid sequence of the first polypeptide chain of TRIDENT-B is the same as that of the first polypeptide chain of the above-described DART-A diabody (SEQ ID NO:113). Similarly, the amino acid sequence of the second polypeptide chain of TRIDENT-B is the same as that of the second polypeptide chain of the above-described DART-A diabody (SEQ ID NO:114).

The third polypeptide chain of TRIDENT-B has the amino acid sequence of SEQ ID NO:119:

QVQLQESGPG LVKPSQTLSL SCTVSGGSSS SGAHYWSWIR
QYPGKGLEWI GYIHYSGNTY YNPSLKSRIT ISQHTSENQF
SLKLNSVTVA DTAVYYCARG TRLRTLRNAF DIWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEA
AGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LSCAVKGFYP SDIAVEWESN GQPENNYKTT
PPVLDSDGSF FLVSKLTVDK SRWQQGNVFS CSVMHEALHN
RYTQKSLSLS PGK

Residues 1-123 of the third polypeptide chain of TRIDENT-B correspond to the VH Domain of the anti-gp120 antibody A32 (SEQ ID NO:96). Residues 124-221 correspond to an IgG1 CH1 Domain (SEQ ID NO:3). Residues 222-236 correspond to an IgG1 Hinge Domain (SEQ ID NO:7). Residues 237-453 correspond to the IgG1 “hole-bearing” CH2-CH3 Domain (SEQ ID NO:50).

The fourth polypeptide chain of TRIDENT-B has the amino acid sequence of SEQ ID NO:120:

QSALTQPPSA SGSPGQSVTI SCTGTSSDVG GYNYVSWYQH
HPGKAPKLII SEVNNRPSGV PDRFSGSKSG NTASLTVSGL
QAEDEAEYYC SSYTDIHNFV FGGGTKLTVL RTVAAPSVFI
FPPSDEQLKS GTASVVCLLN NFYPREAKVQ WKVDNALQSG
NSQESVTEQD SKDSTYSLSS TLTLSKADYE KHKVYACEVT
HQGLSSPVTK SFNRGEC

Residues 1-110 of the fourth polypeptide chain of TRIDENT-B correspond to the VL Domain of the anti-gp120 antibody A32 (SEQ ID NO:97). Residues 111-217 correspond to a CL Kappa Domain (SEQ ID NO:1).

F. gp41×CD16×gp120 Binding Molecule, TRIDENT-C

A third illustrative trivalent gp41×CD16×gp120 Binding Molecule designated “TRIDENT-C” is similar to the above-described TRIDENT-B, but contains the VL and VH Domains of anti-human CD16 antibody h3G8 in lieu of the CD3 mAb 1 (D65G) VL and VH Domains of TRIDENT-B. Thus, TRIDENT-C is composed of four polypeptide chains and possesses one Binding Domain that comprises the VL and VH Domains of the anti-human gp41 antibody 7B2GL (and is thus immunospecific for an epitope of gp41), one Binding Domain that comprises the VL and VH Domains of h3G8 (and is thus immunospecific for an epitope of the CD16 Antigen) and one Binding Domain that comprises the VL and VH Domains of A32 (and is thus immunospecific for an epitope of the gp120 Antigen).

The amino acid sequence of the first polypeptide chain of TRIDENT-C is the same as that of the first polypeptide chain of the above-described DART-B diabody (SEQ ID NO:115). Similarly, the amino acid sequence of the second polypeptide chain of TRIDENT-C is the same as that of the second polypeptide chain of the above-described DART-B diabody (SEQ ID NO:116).

The amino acid sequence of the third and fourth polypeptides of TRIDENT-C are the same as that of the third and fourth polypeptide chains of the above-described TRIDENT-B (SEQ ID NO:119 and SEQ ID NO:120).

G. gp41×CD3×gp41 Binding Molecule, TRIDENT-D

A third illustrative trivalent gp41×CD3×gp41 Binding Molecule, designated “TRIDENT-D,” is similar to the above-described TRIDENT-A, but contains the VL and VH Domains of anti-human gp41 antibody 7B2GL lieu of the TRX2 anti-CD8 VL and VH Domains of TRIDENT-A. Thus, TRIDENT-D is composed of four polypeptide chains and possesses two Binding Domain that comprise the VL and VH Domains of the anti-human gp41 antibody 7B2GL (and is thus immunospecific for an epitope of gp41), one Binding Domain that comprises the VL and VH Domains of hCD3 mAb 1 (and is thus immunospecific for an epitope of the CD3 Antigen).

The amino acid sequence of the first polypeptide chain of TRIDENT-D is the same as that of the first polypeptide chain of the above-described DART-A diabody (SEQ ID NO:113). Similarly, the amino acid sequence of the second polypeptide chain of TRIDENT-D is the same as that of the second polypeptide chain of the above-described DART-A diabody (SEQ ID NO:114).

The third polypeptide chain of TRIDENT-D has the amino acid sequence of SEQ ID NO:121:

QVQLVESGGG LVKPGGSLRL SCAASGFTFS EYYMTWIRQA
PGKGLEWVSY ISKNGEYSKY SPSSNGRFTI SRDNAKNSLY
LQMNSLRAED TAVYYCARAD GLTYFSELLQ YIFDLWGQGT
LVTVSSASTK GPSVFPLAPS SKSTSGGTAA LGCLVKDYFP
EPVTVSWNSG ALTSGVHTFP AVLQSSGLYS LSSVVTVPSS
SLGTQTYICN VNHKPSNTKV DKRVEPKSCD KTHTCPPCPA
PEAAGGPSVF LFPPKPKDTL MISRTPEVTC VVVDVSHEDP
EVKFNWYVDG VEVHNAKTKP REEQYNSTYR VVSVLTVLHQ
DWLNGKEYKC KVSNKALPAP IEKTISKAKG QPREPQVYTL
PPSREEMTKN QVSLSCAVKG FYPSDIAVEW ESNGQPENNY
KTTPPVLDSD GSFFLVSKLT VDKSRWQQGN VFSCSVMHEA
LHNRYTQKSL SLSPGK

Residues 1-126 of the third polypeptide chain of TRIDENT-D correspond to the VH Domain of the optimized anti-gp41 antibody 7B2GL (SEQ ID NO:58). Residues 127-224 correspond to an IgG1 CH1 Domain (SEQ ID NO:3). Residues 225-239 correspond to an IgG1 Hinge Domain (SEQ ID NO:7). Residues 240-456 correspond to the IgG1 “hole-bearing” CH2-CH3 Domain (SEQ ID NO:50).

The fourth polypeptide chain of TRIDENT-D has the amino acid sequence of SEQ ID NO:122:

DIVMTQSPDS LAVSLGERAT INCKSSQTLL YSSNNRHSIA
WYQQKPGQPP KLLIYWASMR LSGVPDRFSG SGSGTDFTLT
ISSLQAEDVA VYYCHQYSSH PPTFGQGTKV EIKRTVAAPS
VFIFPPSDEQ LKSGTASVVC LLNNFYPREA KVQWKVDNAL
QSGNSQESVT EQDSKDSTYS LSSTLTLSKA DYEKHKVYAC
EVTHQGLSSP VTKSFNRGEC

Residues 1-113 of the fourth polypeptide chain of TRIDENT-B correspond to the VL Domain of the anti-gp41 antibody 7B2GL (SEQ ID NO:57). Residues 114-220 correspond to a CL Kappa Domain (SEQ ID NO:1).

H. Alternative Multispecific gp41-Binding Molecules

As will be recognized in view of the instant disclosure, additional multispecific gp41-Binding Molecules having the general structure of any of the above exemplary molecules comprising the VL and VH domains of 7B2GL and comprising a binding site for an alternative ECM and/or HIV-1 antigen may be constructed by employing the VL and VH domains of alternative ECM and/or HIV-1 antibodies in lieu of the VL and VH domains present in the molecules described above. The VL and VH Domains from numerous alternative ECM and HIV-1 binding sites are provided herein, additional VL and VH Domains are known in the art. Similarly, as provided herein, alternative multispecific gp41-Binding Molecules may likewise be constructed incorporating alternative linkers and/or heterodimer promoting domains, particularly those provided herein.

### VIII. Methods of Production

The molecules of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R.A. (1985) “General Method For The Rapid Solid-Phase Synthesis Of Large Numbers Of Peptides: Specificity Of Antigen Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

Antibodies may be made recombinantly and expressed using any method known in the art. Antibodies may be made recombinantly by first isolating the antibodies made from host animals, obtaining their DNA sequence, and using such DNA sequence to express the antibody recombinantly in host cells (e.g., CHO cells). Another method that may be employed is to express the antibody sequence in plants (e.g., tobacco) or in the milk of transgenic animals. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol. Methods 231:147-157). Suitable methods for making derivatives of antibodies, e.g., humanized, single-chain, etc. are known in the art, and have been described above. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

Vectors containing polynucleotides of interest (e.g., polynucleotides encoding the polypeptide chains of the binding molecules of the present invention) can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cell capable of overexpressing heterologous DNAs can be used for the purpose of expressing a polypeptide or protein of interest. Non-limiting examples of suitable mammalian host cells include but are not limited to COS, HeLa, and CHO cells.

The invention includes polypeptides comprising an amino acid sequence of a binding molecule of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

The invention includes variants of the disclosed binding molecules, including functionally equivalent polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly or deleteriously change the functional activity, or use of chemical analogs. Amino acid residues that can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties of charge, size, etc. as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

In one embodiment, a fusion polypeptide is provided that comprises a Light Chain, a Heavy Chain or both a Light and Heavy Chain. In another embodiment, the fusion polypeptide contains a heterologous immunoglobulin constant region. In another embodiment, the fusion polypeptide contains a VH and a VL Domain of an antibody produced from a publicly-deposited hybridoma. For purposes of this invention, an antibody fusion protein contains polypeptide domains that enable the protein to immunospecifically bind both gp41 and an ECM (e.g., CD3, CD16, etc.), and which contains another amino acid sequence to which it is not attached in the native molecule, for example, a heterologous sequence or a homologous sequence from another region (e.g., a deimmunized albumin-binding domain, a Protein A recognition sequence, a peptide tag, etc.).

The present invention particularly encompasses such binding molecules (e.g., antibodies, diabodies, trivalent binding molecules, etc.) conjugated to a diagnostic or therapeutic moiety. For diagnostic purposes, the binding molecules of the invention may be coupled to a detectable substance. Such binding molecules are useful for monitoring and/or prognosing the development or progression of a disease as part of a clinical testing procedure, such as determining the efficacy of a particular therapy. Examples of detectable substances include various enzymes (e.g., horseradish peroxidase, beta-galactosidase, etc.), prosthetic groups (e.g., avidin/biotin), fluorescent materials (e.g., umbelliferone, fluorescein, or phycoerythrin), luminescent materials (e.g., luminol), bioluminescent materials (e.g., luciferase or aequorin), radioactive materials (e.g., carbon-14, manganese-54, strontium-85 or zinc-65), positron emitting metals, and nonradioactive paramagnetic metal ions. The detectable substance may be coupled or conjugated either directly to thebinding molecule or indirectly, through an intermediate (e.g., a linker) using techniques known in the art.

For therapeutic purposes, the binding molecules of the invention may be conjugated to a therapeutic moiety such as a cytotoxin, (e.g., a cytostatic or cytocidal agent), a therapeutic agent or a radioactive metal ion, e.g., alpha-emitters. A cytotoxin or cytotoxic agent includes any agent that is detrimental to cells such as, for example, Pseudomonas exotoxin, Diptheria toxin, a botulinum toxin A through F, ricin abrin, saporin, and cytotoxic fragments of such agents. A therapeutic agent includes any agent having a therapeutic effect to prophylactically or therapeutically treat a disorder. Such therapeutic agents may be may be chemical therapeutic agents, protein or polypeptide therapeutic agents, and include therapeutic agents that possess a desired biological activity and/or modify a given biological response. Examples of therapeutic agents include alkylating agents, angiogenesis inhibitors, anti-mitotic agents, hormone therapy agents, and antibodies useful for the treatment of cell proliferative disorders. The therapeutic moiety may be coupled or conjugated either directly to the binding molecule or indirectly, through an intermediate (e.g., a linker) using techniques known in the art.

### IX. Uses of the Binding Molecules of the Present Invention

As discussed above, molecules capable of binding both gp41 and a ECM are capable of mediating the redirected cell killing of a target cell (i.e., a pathogen-infected cell) that expresses such gp41 on its cell surface. Such molecules may be used for therapeutic purposes, for example in subjects with an infection, particularly a latent HIV-1 infection. Thus, binding molecules of the present invention have the ability to treat a disease or condition associated with or characterized by the expression of gp41, particularly latent HIV-1 infection. Thus, without limitation, the binding molecules of the present invention may be employed in the treatment of an HIV-1 infection, particularly a latent HIV-1 infection.

In particular, the present invention encompasses such methods wherein the molecule capable of binding gp41 comprises an “Epitope-Binding Domain” of an antibody that is capable of binding gp41 and also comprises an Epitope-Binding Domain capable of binding an ECM (in particular CD3, and/or CD16) on the surface of an immune effector cell so as to mediate the redirected killing of the gp41-expressing target cell (for example, by mediating redirected cell killing (e.g., redirected T-cell cytotoxicity)).

In certain aspects the invention provides use of the gp41-Binding Molecules of the invention, particularly multispecific gp41-Binding Molecules such as but not limited to bispecific and trispecific molecules (e.g., bispecific antibodies, bispecific diabodies, trivalent binding molecules, etc.), in methods of treating and preventing HIV-1 infection in an individual, comprising administering to the individual a therapeutically effective amount of a composition comprising a gp41-binding molecule of the invention in a pharmaceutically acceptable form. In certain embodiments, the gp41-binding molecule binds different HIV-1 epitopes, preferably different epitopes present on the HIV-1 envelop.

The various gp41-Binding Molecules described herein have utility, for example, in settings including but not limited to the following:

i) in the setting of anticipated known exposure to HIV-1 infection, a gp41-binding molecule of the present invention can be administered prophylactically (e.g., IV, topically or intranasally) as a microbicide,

ii) in the setting of known or suspected exposure, such as occurs in the setting of rape victims, or commercial sex workers, or in any homosexual or heterosexual transmission without condom protection, a gp41-binding molecule of the present invention can be administered as post-exposure prophylaxis, e.g., IV or topically, and

iii) in the setting of Acute HIV-1 infection (AHI), a gp41-binding molecule of the present invention can be administered as a treatment for AHI to control the initial viral load, or for the elimination of virus-infected CD4 T cells.

In accordance with the invention, the gp41-Binding Molecules of the present invention can be administered prior to contact of the subject or the subject's immune system/cells with HIV-1 or within about 48 hours of such contact. Administration within this time frame can maximize inhibition of infection of vulnerable cells of the subject with HIV.

In addition, various forms of the gp41-Binding Molecules of the present invention can be administered to chronically or acutely infected HIV-1 subjects and used to kill remaining virus infected cells by virtue of these gp41-binding molecule binding to the surface of virus infected cells and being able to mediate redirected cell killing of such infected cells.

In certain embodiments, the gp41-Binding Molecules of the invention can be administered in combination with latency-activating agents, so as to activate a latent reservoir of HIV-infected cells that may be present in a subject. The expectation is that by activating latent proviral HIV-1 DNA in resting cells, previously inactive cells will start producing new virus and will be recognized and eliminated by an immune system that has been augmented by the gp41-Binding Molecules of the invention. Non-limiting examples of latency-activating agents are HDAC inhibitors, e.g., vorinostat, romidepsin, panobinostat, disulfiram, JQ1, bryostatin, PMA, ionomycin, or any combination thereof. See Bullen et al. Nature Medicine 20, 425-429 (2014).

In certain embodiments the gp41-Binding Molecules of the invention can be administered in combination with anti-retroviral agents.

In a specific embodiment, the molecule capable of binding gp41 and the ECM is a bispecific antibody, a BiTe®, or a TandAb®.

In a specific embodiment, the molecule capable of binding gp41 and the ECM is a bispecific diabody.

In a specific embodiment, the molecule capable of binding gp41 and the ECM is a trivalent binding molecule.

As used herein, the terms: “providing a therapy” and “treating” refer to any administration of a composition that is associated with any indicia of beneficial or desired result, including, without limitation, any clinical result such as decreasing symptoms resulting from the disease, attenuating a symptom of infection (e.g., viral load, fever, pain, sepsis, etc.), a decreasing of a symptom resulting from the disease, an increasing of the quality of life of the recipient subject, a decreasing of the dose of other medications being provided to treat a subject's disease, an enhancing of the effect of another medication such as via targeting and/or internalization, a delaying of the progression of the disease, and/or a prolonging of the survival of recipient subject.

Subjects for treatment include animals, most preferably mammalian species such as non-primate (e.g., bovine, equine, feline, canine, rodent, etc.) or a primate (e.g., monkey such as, a cynomolgus monkey, human, etc.). In a preferred embodiment, the subject is a human.

Exemplary disorders that may be treated by various embodiments of the present invention include, but are not limited to, HIV-1 infection (especially a latent HIV-1 infection associated with expression of gp41 bound by a molecule capable of mediating redirected cell killing). In various embodiments, the invention encompasses methods and compositions for treatment, prevention or management of a disease or disorder in a subject, comprising administering to the subject a therapeutically effective amount the binding molecules of the present invention. Such molecules are particularly useful for reducing HIV-1 load, or eliminating HIV-infected cells. Although not intending to be bound by a particular mechanism of action, such molecules may mediate effector function against target cells, promote the activation of the immune system against target cells, cross-link cell-surface antigens and/or receptors on target cells and enhance apoptosis or negative growth regulatory signaling, or a combination thereof, resulting in clearance and/or reduction in the number of target cells.

### X. Pharmaceutical Compositions

The present invention encompasses compositions comprising a gp41-Binding Molecule of the invention. The compositions of the invention include bulk drug compositions useful in the manufacture of pharmaceutical compositions (e.g., impure or non-sterile compositions) and pharmaceutical compositions (i.e., compositions that are suitable for administration to a subject) that can be used in the preparation of unit dosage forms. Such compositions comprise a prophylactically or therapeutically effective amount of a molecule capable of binding gp41 and also capable of binding to an ECM (i.e., a gp41×ECM Binding Molecule) so as to be capable of mediating the redirected killing of a target cell (e.g., an HIV-infected cell, etc.), or a combination of such agents and a pharmaceutically acceptable carrier. Preferably, compositions of the invention comprise a prophylactically or therapeutically effective amount of the binding molecules of the present invention and a pharmaceutically acceptable carrier. In a preferred aspect, such compositions are substantially purified (i.e., substantially free from substances that limit its effect or produce undesired side effects).

Various formulations of such compositions may be used for administration. In addition to the pharmacologically active agent(s), the compositions of the present invention may contain suitable pharmaceutically acceptable carriers comprising excipients and auxiliaries that are well-known in the art and are relatively inert substances that facilitate administration of a pharmacologically effective substance or which facilitate processing of the active compounds into preparations that can be used pharmaceutically for delivery to the site of action. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with a gp41-Binding Molecule of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the binding molecules of the present invention. The kit can further comprise one or more other prophylactic and/or therapeutic agents useful for the treatment of cancer, in one or more containers.

### XI. Methods of Administration

The compositions of the present invention may be provided for the treatment, prophylaxis, and amelioration of one or more symptoms associated with a disease, disorder or infection by administering to a subject an effective amount of a pharmaceutical composition comprising a gp41-Binding Molecule of the present invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Methods of administering a gp41-Binding Molecule or composition comprising such gp41-Binding Molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the binding molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

The invention also provides that preparations of the gp41-Binding Molecule of the present invention are packaged in a hermetically sealed container such as an ampoule or sachette indicating the quantity of the molecule. In one embodiment, such gp41-Binding Molecule are supplied as a dry sterilized lyophilized powder or water free concentrate in a hermetically sealed container and can be reconstituted, e.g., with water or saline to the appropriate concentration for administration to a subject. Preferably, the gp41-Binding Molecule of the present invention are supplied as a dry sterile lyophilized powder in a hermetically sealed container.

The lyophilized preparations of the gp41-Binding Molecule of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such binding molecules, when provided in liquid form, are supplied in a hermetically sealed container.

The amount of such preparations of the invention that will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each recipient subject's circumstances. Effective doses may be extrapolated from dose-response curves derived from in vitro or animal model test systems.

As used herein, an “effective amount” of a pharmaceutical composition is an amount sufficient to effect beneficial or desired results including, without limitation, clinical results such as decreasing symptoms resulting from the disease, attenuating a symptom of infection (e.g., viral load, fever, pain, sepsis, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals. When applied to an individual active ingredient, administered alone, the term refers to that ingredient alone. When applied to a combination, the term refers to combined amounts of the active ingredients that result in the therapeutic effect, whether administered in combination, serially, or simultaneously.

An effective amount can be administered in one or more administrations. For purposes of this invention, an effective amount of drug, compound, or pharmaceutical composition is an amount sufficient: to kill and/or reduce the proliferation of HIV-1 infected cells, to reduce the proliferation of (or the effect of) an HIV-1 virus and to reduce and/or delay the development of the HIV-mediated disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art.

For the binding molecules encompassed by the invention, the dosage administered to a recipient subject is preferably determined based upon the body weight (kg) of the recipient subject. For the binding molecules encompassed by the invention, the dosage administered to a recipient subject is typically from about 0.01 μg/kg to about 300 mg/kg or more of the subject's body weight.

The dosage and frequency of administration of a binding molecule of the present invention may be reduced or altered by enhancing uptake and tissue penetration of the molecule by modifications such as, for example, lipidation.

The dosage of a binding molecule of the invention administered to a recipient subject may be calculated for use as a single agent therapy. Alternatively, the molecule may be used in combination with other therapeutic compositions and the dosage administered to a recipient subject are lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as SIALASTIC® membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 3 17-327).

Treatment of a subject with a therapeutically or prophylactically effective amount of a binding molecule of the present invention can include a single treatment or, preferably, can include a series of treatments. In a preferred example, a subject is treated with a pharmaceutical composition of the invention for between about 1 to 10 weeks, preferably between 2 to 8 weeks, more preferably between about 3 to 7 weeks, and even more preferably for about 4, 5, or 6 weeks. The pharmaceutical compositions of the invention can be administered once a day with such administration occurring once a week, twice a week, once every two weeks, once a month, once every six weeks, once every two months, twice a year or once per year, etc. Alternatively, the pharmaceutical compositions of the invention can be administered twice a day with such administration occurring once a week, twice a week, once every two weeks, once a month, once every six weeks, once every two months, twice a year or once per year, etc. Alternatively, the pharmaceutical compositions of the invention can be administered three times a day with such administration occurring once a week, twice a week, once every two weeks, once a month, once every six weeks, once every two months, twice a year or once per year, etc. It will also be appreciated that the effective dosage of the molecules used for treatment may increase or decrease over the course of a particular treatment.

### EXAMPLES

Having now generally described the invention, the same will be more readily understood through reference to the following examples, which are provided by way of illustration and are not intended to be limiting of the present invention unless specified.

### Example 1

### Optimization

The anti-HIV-1 Env (gp41 protein) antibody designated “7B2 IgG” (GenBank Accession No. AFQ31503; Buchacher, A. et al. (1994) “Generation Of Human Monoclonal Antibodies Against HIV-1 Proteins; Electrofusion And Epstein-Barr Virus Transformation For Peripheral Blood Lymphocyte Immortalization,” AIDS Res. Hum. Retroviruses 10(4):359-369; Shen, R. (2010) “GP41-Specific Antibody Blocks Cell-Free HIV-1 Type 1 Transcytosis Through Human Rectal Mucosa And Model Colonic Epithelium,” J. Immunol. 184(7):3648-3655) was found to comprise numerous amino acid residues in its framework regions that are rare in the human IgG germline repertoire. The presence of such rare non-germline residues can be immunogenic and result in the generation of anti-drug-antibodies upon repeated administration to recipient subjects. To reduce or even eliminate the immunogenic potential of these domains, the 7B2 variable region was optimized by introducing mutations into its framework regions to replace such rare amino acid residues with germline amino acid residues.

As shown in FIGS. 7A-7B, nine (9) mutations were introduced into the VL framework regions (FIG. 7A) and seventeen (17) amino acid substitutions were introduced into the VH framework regions (FIG. 7B) to generate fully germlined VL and VH Domains designated “7B2GL VL” and “7B2GL VII,” respectively. The resulting VL/VH Domains are collectively referred to as “7B2GL.”

The amino acid sequences of the VL Domains of antibody 7B2 and antibody 7B2GL have been presented above (SEQ ID NO:55 and SEQ ID NO:57, respectively). The amino acid sequences of the VL Domain of the framework 1-3 donor (IGKV4-1) and the amino acid sequence of the framework 4 region of the framework 4 donor (IGKJ1-01) are:

IGKV4-1 (SEQ ID NO: 123):
DIVMTQSPDS LAVSLGERAT INCKSSQSVL YSSNNKNYLA
WYQQKPGQPP KLLIYWASTR ESGVPDRFSG SGSGTDFTLT
ISSLQAEDVA VYYCQQYYST P
and
IGKJ1-01 (SEQ ID NO: 124):
FGQGTKVEIK.

The amino acid sequences of the VH Domains of antibody 7B2 and antibody 7B2GL have been presented above (SEQ ID NO:56 and SEQ ID NO:58, respectively). The amino acid sequences of the VH Domain of the framework 1-3 donor (IGVH3-11) and the amino acid sequence of the framework 4 region of the framework 4 donor (IGHJ4-01) are:

IGVH3-11 (SEQ ID NO: 125):
QVQLVESGGG LVKPGGSLRL SCAASGFTFS DYYMSWIRQA
PGKGLEWVSY ISSSSSYTNY ADSVKGRFTI SRDNAKNSLY
LQMNSLRAED TAVYYCAR
and
IGHJ4-01 (SEQ ID NO: 126):
WGQGTLVTVSS

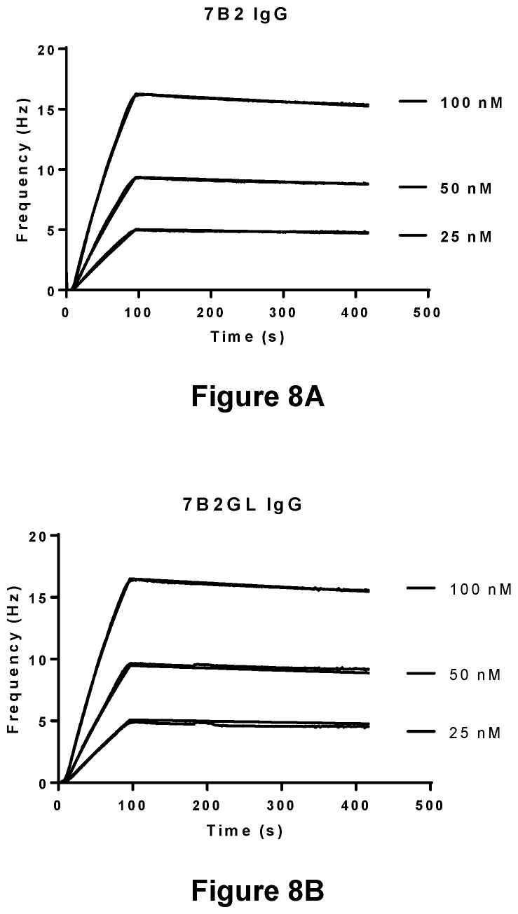

The binding of an IgG comprising the fully germlined variable regions (designated “7B2GL IgG”) was examined by Attana Cell A200 QCM. Briefly, HIV-1 JRFL gp140 Recombinant protein (HIV-1 Env protein minus the transmembrane and C-terminal amino acid residues, but retaining the gp41 epitope recognized by 7B2) at 25, 50 and 100 nM was passed over 7B2 IgG (FIG. 8A) or 7B2GL IgG (FIG. 8B) independently captured on an Attana sensor chip coated with an anti-human IgG Fc surface. For analysis of the kinetic parameters (ka, kd, KD), the 1:1 binding was chosen as the fitting model because it is the least manipulated model mathematically. However, because the JRFL gp140 is a mixture a monomer (20-30%), dimers (40%), and trimers (30%), this model does not fully describe the interactions. Accordingly, the kinetics parameters (ka, kd, KD using 1:1 binding fit) provided in Table 8 are useful primarily for the purpose of directly comparing Ag binding activity of 7B2 and 7B2GL constructs to one another. Although a number of the changes are located in Vernier zones (e.g., VH Domain Kabat positions: 30, 48, 49, and 79, and VL Domain Kabat position 48) that are known to be important for antigen binding (Foote and Winter (1992) “Antibody Framework Residues Affecting The Conformation Of The Hypervariable Loops,” J. Mol. Biol. 224:487-499), and are not typically changed (see, e.g., Chromikova et al. (2015) “Introduction Of Germline Residues Improves The Stability Of Anti-HIV mAb 2G12-IgM,” Biochim. Biophys. Acta 1854:1536-1544), these studies show that all 26 germline mutations were well tolerated and that the fully germlined antibody exhibited epitope binding kinetics that were nearly identical to those of the wild type antibody, 7B2.

TABLE 8

| Antibody | ka | kd | KD (nM) |
|---|---|---|---|
| 7B2 | 7.0E4 ± 3.0E2 | 2.02E−4 ± 4.0E−7 | 2.9 ± 0.01 |
| 7B2GL | 6.9E4 ± 6.0E2 | 2.02E−4 ± 6.0E−7 | 2.9 ± 0.03 |

### Example 2

### HIV×CD3 Binding Molecules

Two HIV×CD3 Binding Molecules capable of binding to the HIV Env gp41 protein and to the exemplary immune effector cells target, CD3, were generated. In particular, two bispecific diabodies (designated “DART-1,” and “DART-A,”) comprising 7B2 or 7B2GL variable domains and have three polypeptide chains were generated, each comprising CD3 mAb 1 (D65G) variable domains. DART-1 and DART-A are Fc Domain-containing, bispecific diabodies having the general structure shown in FIG. 4A that are capable of binding HIV and CD3. The domains and amino acid sequences of these exemplary molecules are described in detail above.

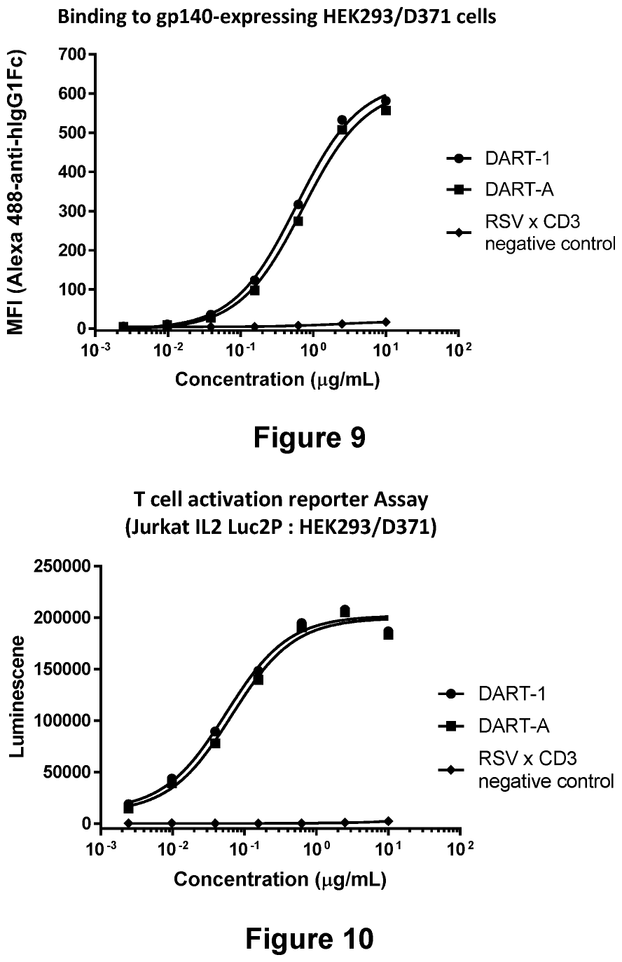

The biological activity of DART-1 and DART-A was examined in a number of assays. The ability of DART-1 and DART-A to bind to gp41 expressed on the surface of HEK/D371 cells was evaluated by flow cytometry. Briefly, gp140-expressing HEK 293/D371 cells (doxycycline-inducible expression of HIV-1 CM244 (subtype AE) gp140 envelope, obtained from Dr. John Kappes (University of Alabama at Birmingham), after 24 hours pre-incubation with 1 pg/mL doxycycline) were incubated with serial dilutions (10 μg/mL-0.0024 pg/mL) of DART-1, DART-A, or the RSV×CD3 negative control. After washing, cells were incubated with Alexa 488-anti-human IgG1 Fc secondary antibody and analyzed by flow cytometery (e.g., using a Guava EasyCyte 8HT (Millipore) and MFI plotted by GraphPad Prism 7). As shown in FIG. 9, DART-1 and DART-A bind to gp140-expressing HEK 293/D371 cells in a similar dose dependent manner.

The ability of DART-1 and DART-A to activate T cells was evaluated using a Jurkat T cell reporter assay (Promega). Briefly, Jurkat IL2 Luc2P reporter cells were co-cultured with gp140 expressing HEK293/D371 cells (20:1) in the presence of serial dilutions (10 μg/mL-0.0024 pg/mL) of DART-1, DART-A, or the RSV×CD3 negative control and luminescence was measured using the ONE-GLO™ Luciferase Assay System (Promega). As shown in FIG. 10, DART-1 and DART-A exhibit comparable T-cell activation activity.

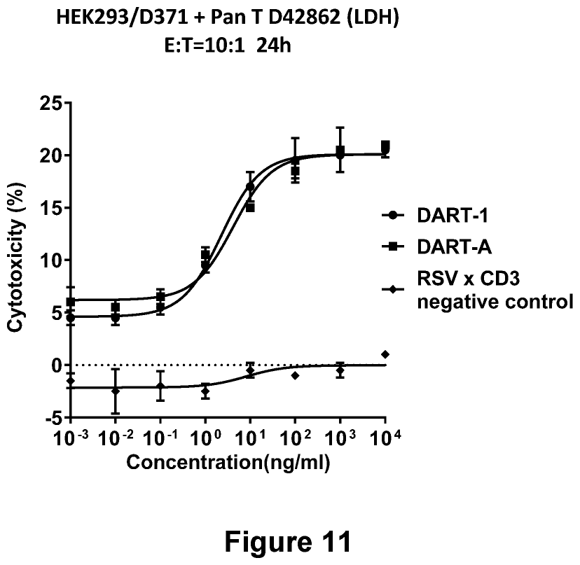

The ability of DART-1 and DART-A to mediate T cell redirected target cell killing was evaluated using a CTL assay. Briefly, Pan T cells are isolated from healthy human PBMCs (e.g., using Dynabeads® Untouched™ Human T Cells Kit (Invitrogen)). gp140 expressing HEK293/D371 (1-4×10⁵ cells/mL) are treated with serial dilutions of DART-1, DART-A, or the RSV×CD3 negative control, together with human T cells at an effector:target (E:T) ratio=10:1, or otherwise at varying E:T ratios as indicated, and incubated at 37° C., 5% CO₂ for 24 hours. Cytotoxicity is measured by lactate dehydrogenase (LDH) release (e.g., using CytoTox 96® Non-Radioactive Cytotoxicity Assay, Promega). As shown in FIG. 11, DART-1 and DART-A exhibit comparable T cell redirected target cell killing activity.

The results of these studies demonstrate that the numerous substitutions introduced into the VL and VH Domains of 7B2 to generate the optimized fully germlined 7B2GL Domains do not impact gp140 binding or biological activity in the above described assays.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A gp41-Binding Molecule comprising a Variable Light Chain (VL) Domain and a Variable Heavy Chain (VH) Domain, wherein the VL Domain comprises the amino acid sequence of SEQ ID NO:57 and/or said VH Domain comprises the amino acid sequence of SEQ ID NO:58.

2. The gp41-Binding Molecule of claim 1, wherein said gp41-Binding Molecule comprises:
(a) said VL Domain that comprises the amino acid sequence of SEQ ID NO:57 and
(b) said VH Domain that comprises the amino acid sequence of SEQ ID NO:58.

3. The gp41-Binding Molecule of any of claims 1-2, wherein said molecule is an antibody or comprises a gp41 epitope-binding portion thereof.

4. The gp41-Binding Molecule of any one of claims 1-3, wherein said molecule is:
(a) a bispecific antibody; or
(b) a diabody, said diabody being a covalently bonded complex that comprises two, three, four or five polypeptide chains; or
(c) a trivalent binding molecule, said trivalent binding molecule being a covalently bonded complex that comprises three, four, five, or more than five polypeptide chains.

5. The gp41-Binding Molecule of claim 4, wherein said molecule is said diabody and comprises an Albumin-Binding Domain (ABD).

6. The gp41-Binding Molecule of any one of claims 1-4, wherein said molecule comprises an Fc Region.

7. The gp41-Binding Molecule of claim 5, wherein said Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

8. The gp41-Binding Molecule of claim 7, wherein said modifications that reduce the affinity of the variant Fc Region for an FcγR comprise the substitution of L234A; L235A; or L234A and L235A, wherein said numbering is that of the EU index as in Kabat.

9. The gp41-Binding Molecule of any one of claim 7 or 8, wherein said modifications that that enhance the serum half-life of the variant Fc Region comprise the substitution of M252Y; M252Y and S254T; M252Y and T256E; M252Y, S254T and T256E; or K288D and H435K, wherein said numbering is that of the EU index as in Kabat.

10. The gp41-Binding Molecule of any one of claims 1-9, wherein said molecule is bispecific and comprises one epitope-binding site capable of immunospecific binding to an epitope of gp41 and one epitope-binding site capable of immunospecific binding to an epitope of a molecule present on the surface of an effector cell.

11. The gp41-Binding Molecule of any one of claims 1-9, wherein said molecule is bispecific and comprises two epitope-binding sites capable of immunospecific binding to an epitope of gp41 and two epitope-binding sites capable of immunospecific binding to an epitope of a molecule present on the surface of an effector cell.

12. The gp41-Binding Molecule of any one of claims 1-9, wherein said molecule is trispecific and comprises:
(a) one epitope-binding site capable of immunospecific binding to an epitope of gp41;
(b) one epitope-binding site capable of immunospecific binding to an epitope of a first molecule present on the surface of an effector cell; and
(c) one epitope-binding site capable of immunospecific binding to an epitope of a second molecule present on the surface of an effector cell.

13. The gp41-Binding Molecule of any one of claims 1-9, wherein said molecule is trispecific and comprises:
(a) one epitope-binding site capable of immunospecific binding to a first epitope of gp41;
(b) one epitope-binding site capable of immunospecific binding to a second epitope of gp41 or an epitope of a different HIV-1 protein; and
(c) one epitope-binding site capable of immunospecific binding to an epitope a molecule present on the surface of an effector cell;
wherein said first and second epitopes of gp41 are different.

14. The gp41-Binding Molecule of any one of claims 10-13, wherein said molecule is capable of simultaneously binding to gp41 and said molecule present on the surface of an effector cell.

15. The gp41-Binding Molecule of any one of claims 10-14, wherein said molecule present on the surface of an effector cell is CD2, CD3, CD8, CD16, TCR, NKp46, or NKG2D.

16. The gp41-Binding Molecule of any one of claims 10-15, wherein said effector cell is a cytotoxic T-cell, or a Natural Killer (NK) cell.

17. The gp41-Binding Molecule of claim 15, wherein said molecule present on the surface of an effector cell is CD3.

18. The gp41-Binding Molecule of claim 12, wherein said first molecule present on the surface of an effector cell is CD3 and said second molecule present on the surface of an effector cell is CD8.

19. The gp41-Binding Molecule of any one of claims 10-18, wherein said molecule mediates coordinated binding of a cell expressing gp41 and a cytotoxic T cell.

20. The gp41-Binding Molecule of any one of claims 1-4 and 6-9 wherein said molecule comprises a first polypeptide chain, a second polypeptide chain and a third polypeptide chain, and wherein:
I) (a) said first polypeptide chain comprises SEQ ID NO:113;
(b) said second polypeptide chain comprises SEQ ID NO:114; and
(c) said third polypeptide chain comprises SEQ ID NO:112;
or
II) (a) said first polypeptide chain comprises SEQ ID NO:115;
(b) said second polypeptide chain comprises SEQ ID NO:116;
and
(c) said third polypeptide chain comprises SEQ ID NO:112.

21. The gp41-Binding Molecule of any one of claims 1-4 and 6-9, wherein said molecule comprises a first polypeptide chain, a second polypeptide chain, a third polypeptide chain, and a fourth polypeptide chain and wherein:
(a) said first polypeptide chain comprises SEQ ID NO:113;
(b) said second polypeptide chain comprises SEQ ID NO:114;
(c) said third polypeptide chain comprises SEQ ID NO:117; and
(d) said fourth polypeptide chain comprises SEQ ID NO:118.

22. The gp41-Binding Molecule of any one of claims 1-4 and 6-9, wherein said molecule comprises a first polypeptide chain, a second polypeptide chain, a third polypeptide chain, and a fourth polypeptide chain and wherein:
I) (a) said first polypeptide chain comprises SEQ ID NO:113;
(b) said second polypeptide chain comprises SEQ ID NO:114;
(c) said third polypeptide chain comprises SEQ ID NO:119; and
(d) said fourth polypeptide chain comprises SEQ ID NO:120;
or
II) (a) said first polypeptide chain comprises SEQ ID NO:115;
(b) said second polypeptide chain comprises SEQ ID NO:116;
(c) said third polypeptide chain comprises SEQ ID NO:119; and
(d) said fourth polypeptide chain comprises SEQ ID NO:120.

23. A pharmaceutical composition that comprises an effective amount of the gp41-Binding Molecule of any of claims 1-22 and a pharmaceutically acceptable carrier.

24. A method to treat or prevent HIV-1 infection in a subject in need thereof comprising administering to the subject a composition comprising any one of the gp41-Binding Molecules of claim 1-22 or the pharmaceutical composition of claim 23 in a therapeutically effective amount.

25. The method of claim 24, wherein said method further comprises administering a latency-activating agent.

26. The method of claim 25, wherein said latency-activating agent is vorinostat, romidepsin, panobinostat, disulfiram, JQ1, bryostatin, PMA, ionomycin, or any combination thereof.
